(19) United States
(12) Patent Application Publication
(10) Pub. No.: US 2026/0083596 A1
(43) Pub. Date: Mar. 26, 2026

(54) Wound Dressing System

(71) Applicant: J&M Shuler Medical, Inc., Athens, GA (US)

(72) Inventors: Michael Simms Shuler, Athens, GA (US); Brett A. Freedman, Rochester, MN (US)

(21) Appl. No.: 19/340,531

(22) Filed: Sep. 25, 2025

Related U.S. Application Data
(60) Provisional application No. 63/698,767, filed on Sep. 25, 2024.

(51) Int. Cl. A61F 13/05; A61F 13/00; A61F 13/02; A61M 1/00

(52) U.S. Cl. CPC A61F 13/05; A61F 13/02; A61M 1/912; A61M 1/966; A61F 2013/00182

(57) ABSTRACT

A negative pressure wound therapy (“NPWT”) dressing is formed from a transparent polymer and includes large-diameter passageways through the dressing for providing suction and irrigation to a wound. The NPWT dressing is formed so as to be non-compressible and non-collapsible to provide low resistance and maintain positive surface pressure at low vacuum settings. The NPWT dressing includes a docking port and suction dome for coupling the dressing to a suction source and an irrigation source. The NPWT dressing may be customizable, disposable, and/or expandable to treat a variety of wounds.

### INCORPORATION BY REFERENCE

This application claims priority to U.S. Provisional Application Ser. No. 63/698,767, filed Sep. 25, 2024, the disclosure of which is incorporated by reference in its entirety.

### TECHNICAL FIELD

This document relates to wound dressings and wound dressing systems for providing patient wound care with reduced ingrowth of tissue.

### BACKGROUND

Negative-pressure wound therapy (NPWT) is a type of treatment used by physicians to promote the healing of acute or chronic wounds. For example, a sealed wound dressing connected to a vacuum pump can be placed onto an open wound for applying sub-atmospheric pressure to the wound. Such types of negative-pressure applications can be used to draw out fluid from the wound and increase blood flow to a wound area.

### SUMMARY

In an aspect, a dressing for use in a NPWT assembly includes a wound contact layer formed from a transparent material and a sealing layer comprising an adhesive.

In an aspect, a wound dressing includes a transparent polymeric wound contact layer that has a wound-facing surface, a top surface opposite the wound-facing surface, and a docking port formed in the top surface. The docking port includes a central suction hub, and the central suction hub is coupled to a plurality of passageways extending through the wound contact layer between the wound-facing surface and the top surface.

In an aspect, a wound dressing includes a transparent polymeric wound contact layer and at least one coating formed on a wound-facing surface of the transparent polymeric wound contact layer, the at least one coating comprising a therapeutic substance.

In an aspect, a method of treating a wound includes positioning a wound contact layer in a wound, engaging a docking port of the wound contact layer with a suction dome to connect the wound contact layer to a suction source and irrigation source, and providing at least one of suction and irrigation to the wound through the suction dome.

In an aspect, an electronic vacuum regulator system includes a base. The base includes a housing, an inflow port, an outflow port, and a pump apparatus positioned within the housing. The pump apparatus provides suction through the inflow port. The electronic vacuum regulator system also includes a control system coupled to the base, wherein the control system is detachable from the base. The control system controls suction provided by the pump apparatus through the inflow port.

In an aspect, an electronic vacuum regulator (EVR) system includes a housing and a receptacle formed in the housing, the receptacle sized and configured to receive a canister of medicament for distribution to a wound dressing through the housing. In an aspect, a NPWT dressing assembly for use in multi-cavity wound includes a plurality of dressings including at least a first dressing and a second dressing, each of the plurality of dressings including a wound contact layer comprising a polymer material and an irrigation pathway extending throughout the wound contact layer. The first dressing of the plurality of dressings is coupled to the second dressing by an irrigation tube connecting the irrigation pathway of the first dressing to the irrigation pathway of the second dressing.

In an aspect, an expandable wound dressing system includes a wound dressing. The wound dressing includes a dressing including a transparent polymer intended to be inserted into a wound and an expander intended to change at least one dimension of the dressing while the dressing is positioned within the wound.

In an aspect, an expandable wound dressing system intended to be positioned within a wound includes a central shaft and a plurality of ribs extending radially and orthogonally from the central shaft, the plurality of ribs movably coupled to the central shaft at a distal end of the central shaft, and an expander operably connected to the plurality of ribs and intended to change an angle of the plurality of ribs relative to the central shaft.

In an aspect, an expandable wound dressing system includes a bladder including a transparent polymer and a pump intended to inflate and deflate the bladder.

In an aspect, a method of treating a wound using an expandable wound dressing includes positioning an expandable wound dressing within a wound while the expandable wound dressing is in a collapsed state, the wound defined by one or more walls. The method also includes expanding the expandable wound dressing within the wound to an expanded state, and incrementally collapsing the expandable wound dressing as the wound heals.

In an aspect, an expandable wound dressing system includes a bladder, a pump capable of inflating and deflating the bladder, and a plurality of dressings extending around the bladder. Each of the plurality of dressings are formed as an elongate arm with a transparent polymeric dressing coupled to a distal end of the elongate arm.

In an aspect, a NPWT assembly includes a suction dome. The suction dome includes a mounted section, a tubing connection for coupling to tubing of a wound contact layer, and one or more ports extending from the mounded section, the one or more ports intended to be coupled to an external gas or fluid source.

In an aspect, a disposable dressing system includes a wound dressing. The wound dressing includes an adhesive layer, the adhesive layer including a transparent material coated on a first side with an adhesive material at least at a peripheral edge, and the adhesive material is covered by a removable backing paper The wound dressing also includes a wound contact layer, the wound contact layer including a transparent polymeric material having a plurality of passageways therethrough. The wound dressing also includes a port formed through the adhesive layer to the wound contact layer, where an inlet of the port in fluid communication with the plurality of passageways of the wound contact layer. The wound contact layer is coupled to the adhesive layer at the port and is otherwise separable from the adhesive layer.

In an aspect, a kit for treating a wound includes a wound dressing of any type described in this application. The kit further includes a pump for providing suction to the wound dressing and the pump is battery-operated and provides a low flow rate.

In an aspect, a kit for treating a wound includes a wound dressing. The wound dressing includes a wound contact layer and an adhesive layer. The wound contact layer and the adhesive layer are coupled to one another at least at a first portion of the wound contact layer, and the wound contact layer is separable from the adhesive layer at a second portion of the wound contact layer.

In an aspect, a negative pressure wound therapy system includes a vacuum source, a wound interface dressing, and an internal flow distribution structure. The vacuum source is capable of generating a sub-atmospheric pressure of −30 mmHg to −90 mmHg. In some embodiments, the vacuum source is configured for use in intermittent therapy where an increased or maximum sub-atmospheric pressure up to −90 mmHg is generated, followed by generating a low (e.g., less than −5 mmHg) or zero pressure. This enables alternating pressure between increased or maximum values and decreased or minimum values (e.g., low or zero pressures). In some embodiments, the vacuum source can generate sub-atmospheric pressures between −5 mmHg and −70 mmHg, or more specifically between −15 mmHg and −60 mmHg, or more specifically between −20 mmHg and −50 mmHg. The wound interface dressing is formed from a non-compressible low-durometer material that resists collapse under such sub-atmospheric pressures. The internal flow distribution structure maintains a tissue-facing surface pressure +10 mmHg to +50 mmHg. The negative pressure wound therapy system operates in continuous or intermittent vacuum cycles to preserve tissue perfusion.

Some or all of the embodiments detailed herein may have one or more of the following advantages. First, some embodiments provide a dressing in which some or all layers are partially or completely transparent. A transparent NPWT dressing allows a medical professional to have visual access to the wound bed without removing the dressing from the wound, allowing monitoring of the wound's appearance and progress of the treatment. Monitoring the wound without requiring removal of the dressing can reduce trauma to the wound because the dressing can be left in place for longer intervals. Pulling a conventional dressing from a wound to view the wound bed can result in disruption of healing and can be painful to the patient if there has been tissue ingrowth into the dressing or adhesion of blood, scabs, or other fluids to the dressing. By enabling visual monitoring without removal of the dressing from the wound, treatment and healing can continue uninterrupted and the dressing can be used with the wound for a longer duration compared with conventional dressings that need to be periodically removed to view or treat the wound. Embodiments including dressings that are transparent or translucent to technologies like IR or visible light can additionally allow therapeutic treatments to be delivered to the wound bed through the dressing without requiring removal.

Second, some embodiments provide a dressing having a layer adjacent the wound surface formed from a material that can reduce the occurrence of ingrowth of tissue into the dressing. Prevention or reduction of ingrowth into the dressing can allow for the removal of the wound dressing from the wound with less or no disruption of progress toward healing, thus allowing a wound to heal faster. A dressing having reduced ingrowth of tissue can be worn by a patient for a longer period of time before it needs to be removed or changed, increasing the speed that the wound heals. Tissue ingrowth into a dressing can contribute to infection and inflammation of the wound, and the use of a dressing that reduces tissue ingrowth can also reduce the risk of infection arising from the wound retaining parts of the dressing after removal. Additionally, removal of a wound dressing without ingrowth into the dressing can be less painful or uncomfortable to the patient and may improve patient compliance with treatments and wound care schedules.

Third, some embodiments provide a dressing having a structure that includes large passageways that allow for easier flow of fluid, such as air, gas or liquid, through the dressing to or from the wound bed. Large passageways can allow for easier flow and reduce the power needs for the negative pressure pump driving the system. Additionally, large passageways can allow for more light (natural or UV) to reach the wound bed, allowing for enhanced visualization of the wound bed and further enabling the use of light-activated therapeutic compounds in the wound bed.

Fourth, some embodiments provide a wound contact layer of a dressing having at least one coating on the wound-facing surface. The coating can be a therapeutic substance such as an antimicrobial agent, medicaments, biological growth factors, skin supplements, chemotherapeutic agents, or other suitable substances to provide therapeutic effects to the wound. Multiple coatings can be applied to the wound contact layer to allow for timed release of specific therapeutic compounds as a top layer of the coating is worn off and a new coating exposed. Timed release of therapeutic compounds through the use of multiple coatings can provide wound care that is appropriate for the particular phase of healing without requiring that the dressing be removed.

Fifth, some embodiments provide an electronic vacuum regulator (EVR) for use in out-patient environments with features promoting patient compliance. For example, the EVR can include location tracking capabilities that allow a facility to identify the location of a particular EVR device for retrieval when a patient has completed therapy. The EVR can also include communication with a medical facility and provider to update the provider about the use of the EVR, including duration of use, timing of use, and use of the EVR with particular therapeutics. By directly reporting statistics related to therapeutic use of the EVR to the medical facility, the EVR promotes open communication between patient and provider and allows the provider to intervene if the patient is not using the EVR for the recommended therapies. In some implementations, the EVR can be used with therapeutic cartridges that are color-coded, identified by shape, or identified by a barcode or label to allow ease of identification and use by patients and caregivers and to prevent or reduce errors in administration. The EVR can be used in the in-patient setting and utilize wall suction which is readily available in most if not all patient rooms or treatment areas.

Sixth, in some embodiments, a collection canister is provided that is formed from a flexible bag to allow for folding for easy transportation of the collection canister or for use in non-hospital/medical environments. The collection canister can use tortuous pathways through absorbent materials to dry out air passing through the collection canister and can utilize multiple right-angles in the pathway to encourage separation of fluid from gas regardless of an orientation of the collection canister. This allows the collection canister to be used in environments where it is not possible or practical to ensure a particular orientation.

Seventh, some embodiments provide a dressing designed to provide chemotherapeutic agents to a tumor bed. By delivering chemotherapeutic agents that target cancer cells, the dressing can be used to heal a tumor bed and remove any additional cancer cells left behind in the margins of the wound. Additionally, by providing the chemotherapeutic agent to a negative pressure dressing, the exudate removed from the tumor bed by suction can be monitored to determine if additional cancer cells are present in the tumor bed.

Eighth, some embodiments provide a dressing that is designed to expand to fill a tunnel wound and be collapsed within the tunnel wound as the wound heals from the outside in. Such embodiments provide various methods for incremental collapse of the dressing within the wound and extraction of the dressing as the wound heals. The use of such dressings improves the healing of tunnel wounds by reducing the occurrence of abscesses that form when a wound closes around an empty or “dead” space. Dressings that can be collapsed as the wound heals can also include coatings that provide therapies to the surfaces of the wound and devices such as pressure sensors and UV lights that help to determine a healing phase of the wound.

Ninth, some embodiments provide a docking port formed in a dressing to engage with a suction dome for providing suction and, in some implementations, irrigation to a wound. By forming the docking port in the dressing, the connection of the dressing to suction and irrigation is streamlined by requiring a medical professional to only connect the suction dome to the docking port. The suction dome provides an air-tight seal for providing suction to the wound.

Tenth, some embodiments provide a dressing that is customizable in size to fit a shape of a wound. The dressing can include perforations for reducing a size of a wound contact layer or can include irrigation and suction pathways that can be cut at any location while maintaining functionality. In some implementations, the dressing can be formed so that a wound contact layer is connected to an adhesive sealing layer at one end and is separable at another end so that the wound contact layer and the sealing layer can be separately cut through to appropriately size the dressing. The sealing layer needs to be cut so that it extends beyond the edges of the wound contact layer to provide an air-tight seal so that negative pressure can be applied to the dressing. By providing a mechanism for separately sizing the wound contact layer and the sealing layer, the dressing can be easily customized by a medical professional so that the dressing is sized to the wound.

Eleventh, some embodiments provide a disposable dressing and disposable dressing system. Disposable dressings can be applied in a hospital setting and monitored or treated in non-hospital settings with low-power or small pump devices or without the need for a larger or more expensive pump. The disposable dressing and small pump can provide negative pressure or suction to the wound, and the wound can be monitored through the transparent dressing. The disposable dressing can include an opening with a filter for providing airflow to the wound bed to keep the wound bed and skin dry and reduce infection. The suction tubing for the disposable dressing system can include absorbent materials to remove liquid suctioned from the wound before it reaches the pump. By providing a disposable dressing and system (such as a kit), wound therapy can be provided in non-hospital settings without the need for bulky and expensive equipment.

The details of one or more embodiments of the invention are set forth in in the accompanying drawings and the description below. Other features, objects, and advantages of the invention will be apparent from the description and drawings, and from the claims.

### DESCRIPTION OF DRAWINGS

FIG. 1 illustrates a side view of an exemplary negative pressure wound therapy (NPWT) system in a wound bed.

FIG. 2 illustrates a view of an exemplary irrigation pathway of an NPWT system.

FIGS. 3A-H illustrate views of an exemplary docking port and suction dome.

FIGS. 4A-C illustrate steps of a method for using an exemplary wound dressing to treat a tumor bed.

FIG. 5 illustrates an exemplary expandable bladder for treating a wound.

FIG. 6 illustrates an exemplary expandable bladder for use in a wound bed.

FIGS. 7A-E illustrate exemplary shapes of an expandable bladder for use in a wound bed.

FIG. 8 illustrates an exemplary NPWT dressing including multiple extensions positioned in a wound bed.

FIG. 9 illustrates the exemplary NPWT dressing of FIG. 8 partially extracted from a wound bed.

FIGS. 10A-C illustrate views of an exemplary expandable bladder during insertion into a tunnel wound.

FIGS. 11A-C illustrate views of an exemplary expandable bladder during extraction from a tunnel wound.

FIGS. 12A-C illustrate views of an exemplary expandable tunnel wound dressing during insertion into a tunnel wound.

FIGS. 13A-C illustrate views of an exemplary customizable NPWT dressing for use in out-patient settings.

FIG. 14 illustrates a cross-sectional view of an exemplary connection of suction tubing with an NPWT dressing.

FIG. 15 illustrates an exemplary incisional NPWT dressing including a port and tubing for providing suction to the wound.

FIG. 16 illustrates an exemplary incisional NPWT dressing including an opening in the dressing for providing airflow to the wound.

FIGS. 17A-B illustrate side views of an exemplary air flow tube for use with an incisional NPWT dressing.

FIGS. 18A-G illustrate views of a customizable NPWT dressing for use with an incisional wound.

FIG. 19A-C illustrate views of exemplary openings for providing and controlling air flow to a wound through a dressing.

FIGS. 20A-J illustrate shapes of projections from an NPWT dressing to space the dressing away from a wound bed.

FIG. 21 illustrates an exemplary perforated NPWT dressing.

FIGS. 22A-B illustrate exemplary NPWT dressings with positionable suction pads.

FIG. 23 illustrates a mechanism for attaching a dressing component to another component.

FIGS. 24A-H illustrate an alternative configuration for an exemplary suction dome.

FIGS. 25A-C illustrate an exemplary external pad configured to cover dressings and tubing to prevent potential pressure injuries to patients.

FIGS. 26A-C illustrate an exemplary expandable tunnel wound dressing during insertion into a tunnel wound or removal from the tunnel wound.

FIG. 27 illustrates an exemplary NPWT suction system with a compressible suction cannister.

FIGS. 28A-C illustrate an exemplary NPWT suction system with a bridging material and a pressure-reducing layer placed over a dressing covering a wound.

FIG. 29A illustrates a dressing inserted into a wound.

FIG. 29B illustrates a pair of suction domes placed over exposed ends of the dressing of FIG. 29A.

Like reference symbols in the various drawings indicate like elements.

### DETAILED DESCRIPTION OF ILLUSTRATIVE EMBODIMENTS

### I. Description of the Dressing and Dressing System

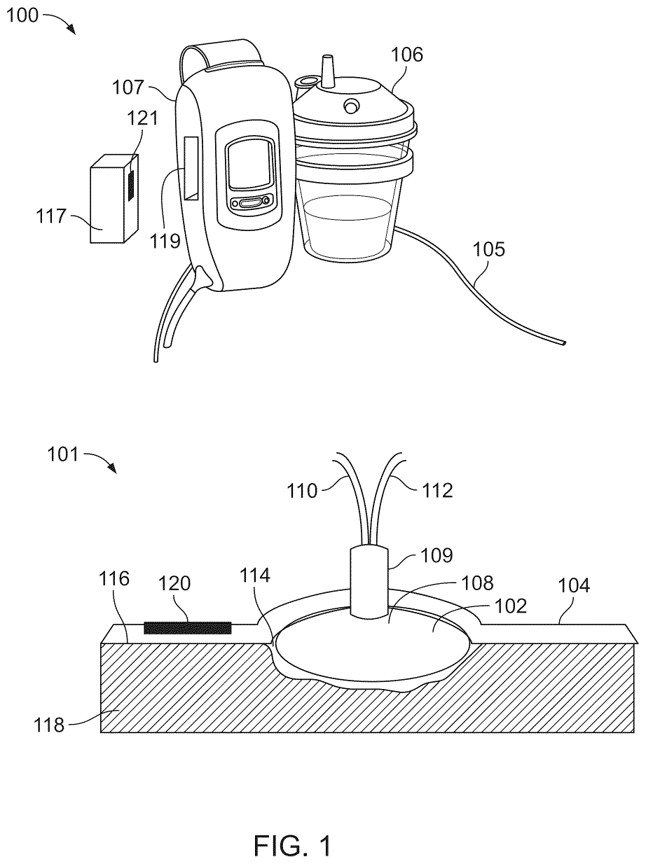

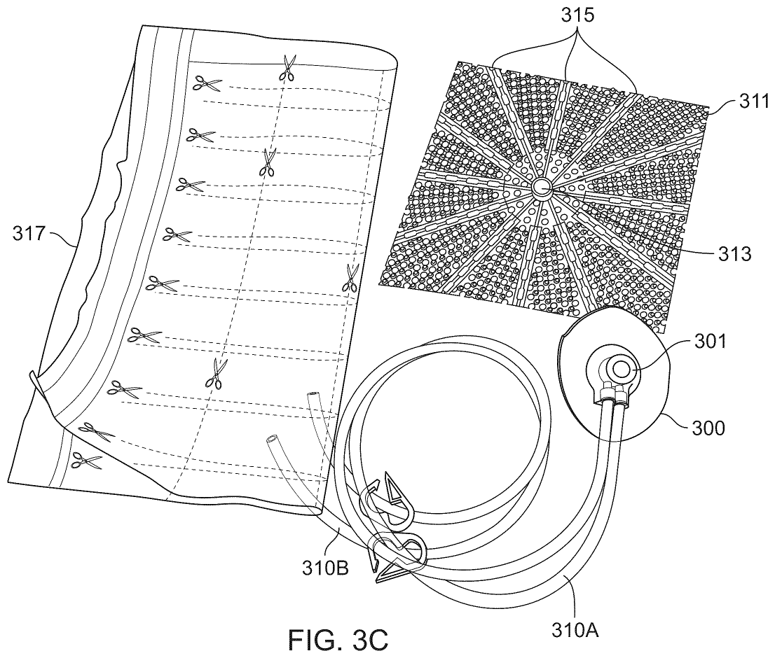

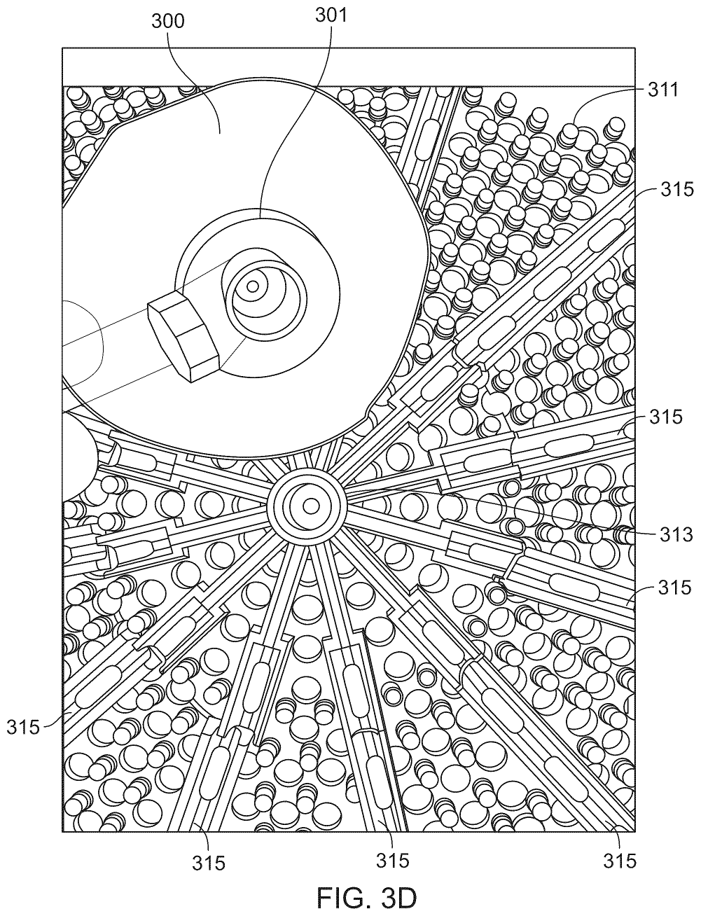

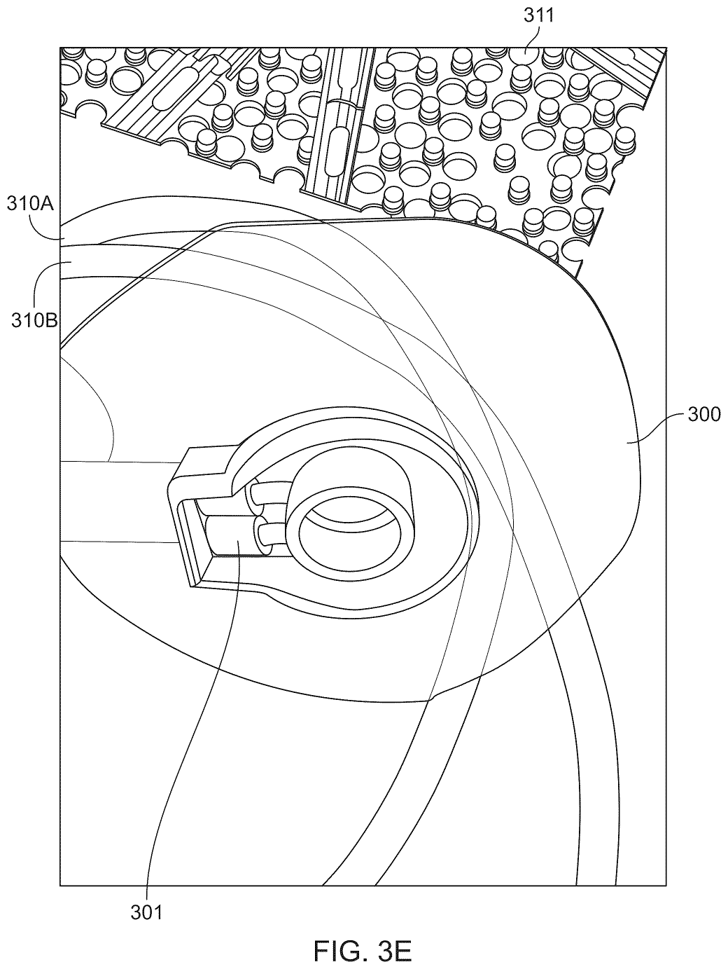

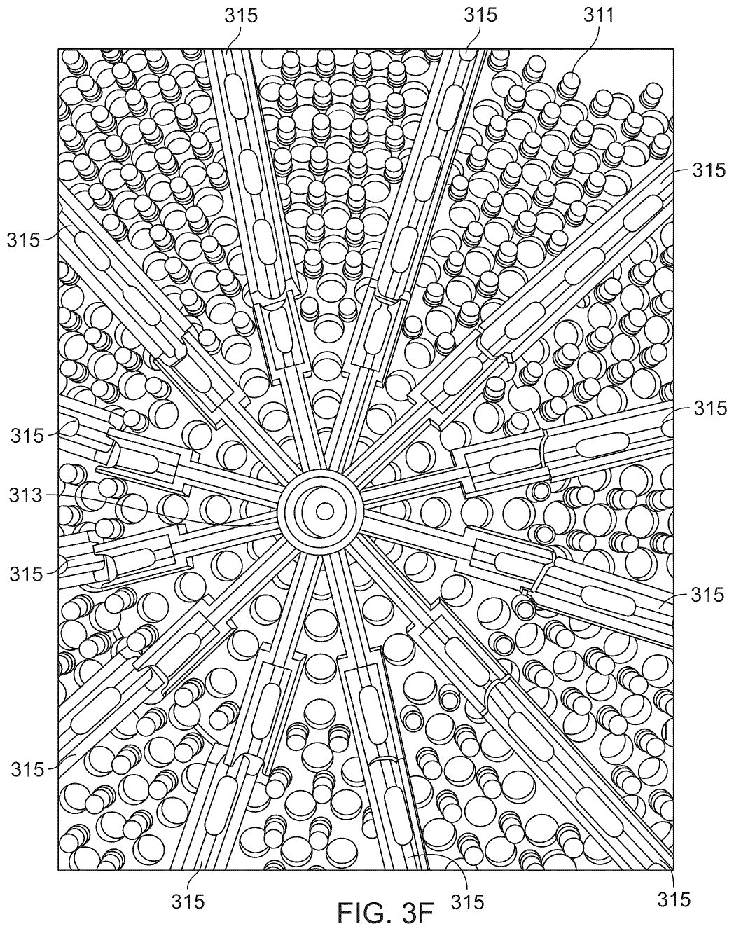

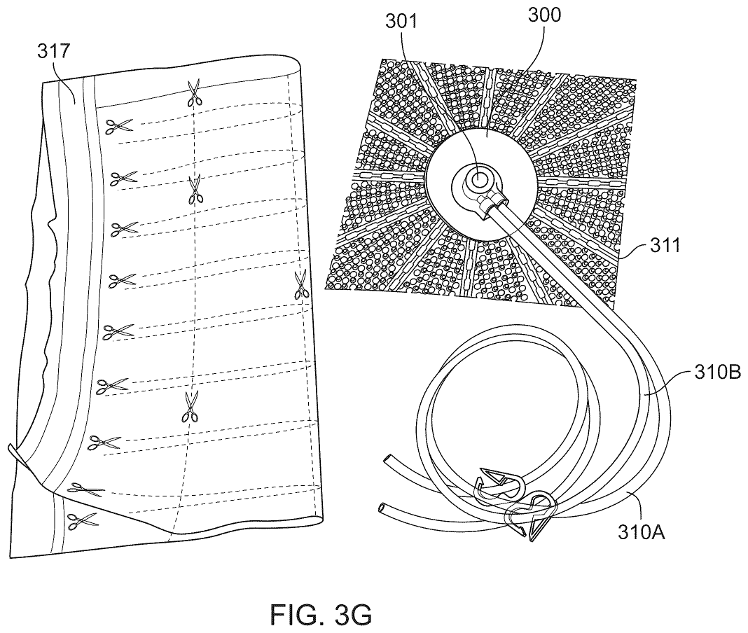

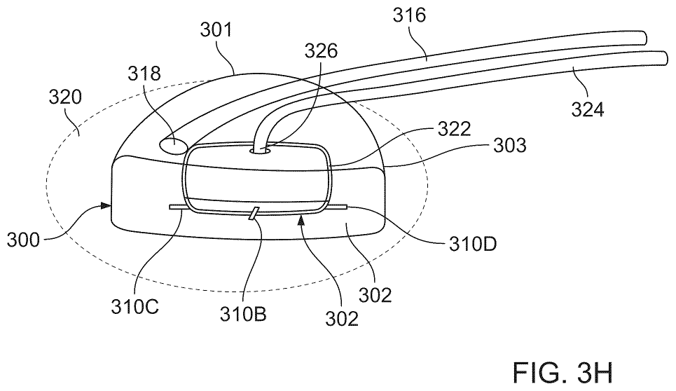

Referring to FIG. 1, an exemplary negative pressure wound therapy (NPWT) system 100 includes a wound dressing 101, a collection canister 106 and a programmable electronic vacuum regulator (EVR) 107. The wound dressing 101 includes a wound contact layer 102 and a sealing layer 104 positioned over the wound contact layer 102. The wound dressing 101 also includes a suction dome assembly 109 coupled to the wound contact layer 102 at a port 108, and inlet tubing 110 and outlet tubing 112 extending from the suction dome assembly 109. As will be described below with regard to FIGS. 3A-H, suction dome assembly 109 includes docking port 300 and suction dome 301. These components are described in further detail, in passages throughout this specification.

In use, the wound dressing 101 is positioned so that the wound contact layer 102 is within a wound bed 114 formed in a patient's tissue 118. The sealing layer 104 extends over the wound contact layer 102 and extends over a skin surface 116 surrounding the wound bed 114. The inlet tubing 110 includes one or more fluid pathways for providing gas and liquid to the wound bed 114 through the wound contact layer 102. The inlet tubing 110 and outlet tubing 112 have separate lumens and can function independent of each other and can also function simultaneously. The outlet tubing 112 includes at least one fluid pathway for removing gas and liquid from the wound bed 114. The outlet tubing 112 can be connected to an electronic vacuum regulator source (e.g., 107) or be connected to a manual pump (e.g., a “sump pump”) that is manually driven. Outlet tubing 112 could also terminate in an open tube to the environment allowing gravity to suction the outflow in the setting of mass casualties or combat areas or wilderness where biohazards can be dispensed onto the ground. The outlet tubing 112 is communicatively coupled to the collection canister 106 by tubing 105. One or both of inlet tubing 110 and outlet tubing 112 is communicatively coupled to the EVR 107. Additionally, outlet tubing 112 is communicatively coupled to a source of suction, such as wall suction or a portable vacuum suction pump. Although FIG. 1 illustrates the wound contact layer 102 with sealing layer 104 extending over the top, in some implementations, wound contact layer 102 includes an adhesive apron for sealing to the skin surface 116 surrounding the wound bed 114 and can be used without sealing layer 104. In such implementations, a top surface of the wound contact layer lacks any passageways or openings other than those associated with the suction dome assembly 109.

In some implementations, the sealing layer 104 is sized to overlap the periwound (e.g., area at the perimeter of the wound where new intact skin is forming). Using conventional dressings, the periwound is traditionally left uncovered so it can be protected from retained moisture in sponge-like dressings to prevent skin maceration that would inhibit tissue healing. In some implementations, the wound contact layer 102 is non-absorptive, and the sealing layer 10 may be optionally omitted or sized to cover the tissue of the periwound.

The wound contact layer 102 can be formed from a thermoplastic elastomer (TPE) or other polymer material to support advanced wound care. In some implementations, the wound contact layer 102 is formed from a polyurethane elastomer, silicone, or a composite material. In some implementations, the wound contact layer 102 is formed with an internal architecture including one or more of a mesh, a lattice, bumps, protrusions, ridges, tunnels, valleys, or a honeycomb structure. In some implementations, the wound contact layer 102 can be formed from another soft, low durometer, medical grade polymer. In some implementations, the wound contact layer 102 is formed from a non-compressible, low-durometer material. The wound contact layer 102 provides low resistance by being non-compressible and non-collapsible, and as a result is capable of achieving and maintaining positive surface pressures (+10 to +100 mmHg) at the wound surface to improve tissue perfusion and avoid ischemia. For example, the wound contact layer formed of a non-compressible, low-durometer material resists collapse under sub-atmospheric pressures in the range of −30 mmHG to −90 mmHg provided by a pump, and the internal flow distribution structure including large-diameter (0.5 mm to 10 mm or greater) flow passageways through the wound contact layer 102 maintain tissue facing surface pressures in the range of +10 mmHg to +100 mmHg. In some implementations, the pump can alternate between an increased or maximum sub-atmospheric pressure up to −90 mmHg and a low (e.g., less than −5 mmHg) or zero pressure. This enables intermittent therapy by alternating sub-atmospheric pressure between increased or maximum values and decreased or minimum values (e.g., low or zero pressures). In some implementations, the vacuum source can generate sub-atmospheric pressures between −5 mmHg and −70 mmHg, or more specifically between −15 mmHg and −60 mmHg, or more specifically between −20 mmHg and −50 mmHg. Similarly, the tissue facing surface pressures may alternate between increased or maximum values (e.g., greater than +50 mmHg) and a low (e.g., less than +5 mmHg) or zero pressure. In some implementations, the tissue facing surface pressure can be between +5 mmHg and +50 mmHg, or more specifically between +10 mmHg and +40 mmHg, or more specifically between +15 mmHg and +35 mmHg. In some implementations, the sub-atmospheric pressures are provided during intermittent vacuum cycles.

By controlling the surface pressure and resisting collapse, the system preserves new tissue, and may support faster, less traumatic healing. Low positive pressure (e.g., between +5 mmHg and +40 mmHg) has been shown to increase perfusion which can promote healing for the patient. Other examples of this include athletic compression sleeves which promote increased blood flow under the sleeve to the user.

A semi-rigid conformable wound contact layer such as one formed by a soft (e.g., low durometer) polymer can have a number of advantages. For example, the lack of compression can be improved substantially in some implementations that use a semi-rigid conformable wound contact layer. If the three-dimensional structure of the dressing avoids collapse under negative pressure, then flow pathways will not be disrupted or closed off during use. Some conventional foam dressings may compress under the positive pressure facilitated by the suction force (negative pressure), which can result in increased resistance to flow. This increased resistance to flow can require the application of a large amount of negative pressure to the foam in order to remove the viscus tissue fluid (e.g., exudate). The increased negative pressure can result in higher levels of compression and flow resistance in some implementations.

In some implementations, when a negative pressure is applied to a dressing, air can be removed (e.g., cussed out) and a positive pressure can be applied to the dressing and the wound surface by extension. This positive pressure can cause the foam to contract in size or compress/collapse down. Reticulated open-cell foam (ROCF) is one example of a foam that may collapse when placed under negative pressure. In the compressed state, the foam can naturally exert a force in an attempt to return to its expanded state. That reciprocal force can increase the positive pressure applied to the wound surface. This phenomenon can create an exponential force; for example, as the negative pressure applied by the vacuum or suction pump is increased, the increased force due to the wound filler collapse or attempt to expand can be exponentially increased. This can result in even higher positive pressures resulting in ischemic conditions under the NPWT dressing in some circumstances.

The semi-rigid construction of a polymer dressing can also have a number of advantages when negative pressure is applied in regard to positive pressure transmission. The polymer dressing can be non-compressible so that the exponential increase in attempting to expand does not occur, or is reduced. Additionally, with a semi-rigid, partially soft wound contact layer, the force can be spread out over a larger area than in other implementations. This can be advantageous in reducing the positive pressure applied to the wound surface. This phenomenon is not found when using ROCF in at least some circumstances.

In some implementations, the wound contact layer 102 is non-compressible but molds or conforms to the size and shape of a wound. In some implementations, the wound contact layer 102 is non-absorptive. Retained body fluid can create a moist environment with nutrients taken from the biologic material which can enable bacteria proliferation. This bacterial overgrowth can result in significant malodor (e.g., an unpleasant smell). The wound contact layer 102 formed from a polymeric material is less likely to retain fluid than a conventional dressing material (such as a fabric or reticulated open-cell foam), reducing the occurrence of bacterial overgrowth resulting from stagnated fluids lying in direct contact with the wound bed 114. This can also reduce bacterial load, reduce various smells, and provide improved healing of open wounds or biological tissue.

The avoidance of a porous material in the wound contact layer 102, like reticulated open-cell foam sponge or even cotton gauze, can reduce the potential for wound tissue ingrowth into the dressing material. Because tissue ingrowth is reduced, the wound dressing 101 can remain in the wound bed 114 for longer durations. Additionally, the polymeric and non-porous wound contact layer 102 can reduce traumatic avulsion of healing wound tissue at each dressing change because there is reduced or no ingrowth of tissue, so wounds can heal faster. Typically dressings are changed every 2 to 3 days because of the potential for tissue ingrowth with conventional dressings. Extending the time between changes of dressing with a wound contact layer 102 resistant tissue ingrowth reduces the trauma to the wound bed 114 caused during dressing changes. The wound contact layer 102 formed from a polymer material also significantly reduces or eliminates particle retention in the dressing, which can arise using conventional porous foam or sponge-based negative pressure wound therapy dressings. Tissue ingrowth using conventional dressings can secure ends of the conventional dressing in the wound tissue so tightly that during dressing removal parts of the porous foam/sponge are retained in the wound, where they can act as nidus for infection or chronic inflammation. Retention of conventional dressings in the wound can lead to heterotopic ossification and other poor wound outcomes, since they are retained foreign bodies. Using a wound dressing 101 with a wound contact layer 102 formed from polymeric tissue-ingrowth resistant material, allows for faster wound healing and overcomes these known and clinically significant weaknesses of conventional porous foam/sponge dressings.

The wound contact layer 102 can be clear, transparent, or translucent in appearance, to allow for direct observation of the patient tissue 118 and wound bed 114 lying below the wound dressing 101. Observation of the wound bed 114 through the wound dressing 101 can be by direct visual observation or can be by technology enhanced (i.e. IR or other light-mediated technology) means for qualitatively assessing wound health and/or quantitatively assessing the bioburden concentration on the surface of the wound bed 114. By allowing observation of the wound bed 114, the transparent or translucent wound contact layer 102 allows a medical professional to observe a status of the wound.

The wound contact layer 102 includes multiple large-diameter passageways formed through the wound contact layer 102. These passageways can be fully formed or be formed through layers of the dressing that once placed under suction create passageways. In some implementations, the passageways range in diameter from 0.5 mm to 5 mm for distribution of negative pressure or distribution of therapeutic agents to the wound. In some implementations, the passageways are variable in size, such that some passageways or areas of passageways are larger than others. In other implementations, all passageways are a same diameter or are within a range of diameters. In some implementations, the diameter of the passageways is in a range between 0.5 mm to 1 mm, 1 mm to 2.5 mm, 2.5 mm to 5 mm, or larger than 5 mm. The large-diameter passageways provide significant empty space within the wound contact layer 102. Further, the large-diameter passageways allow for flow through the dressing with minimal resistance and with no or reduced compression of the passageways and wound contact layer under suction. The large-diameter passageways are fluidically coupled to the port 108. The wound contact layer 102 with large-diameter passageway structure can be 3-D printed or injection molded. In some implementations, the entire dressing is injection molded as a single dressing. The large-diameter passageways through the wound contact layer 102 allow for easier flow through the wound contact layer 102 (including fluid delivery to the wound bed 114 and vacuum removal of fluids or gases from the wound bed) and can reduce the power needs for a negative pressure pump driving the system. The large-diameter passageways provide flow pathways for providing negative pressure and removal of exudate, and for delivery of therapeutics or irrigation to the wound bed 114. The large-diameter passageways through the wound contact layer 102 also allow compression to be provided to the wound bed 114 in a controlled manner. In some implementations, the dressing is non-compressible to protect underlying tissue or is compressible to exert a positive force on the wound surface to promote hemostasis or cell ischemia. In some implementations, a non-compressible dressing is affixed to the tissue surrounding the wound (e.g., using staples or sutures) and wound-up or contracted to exert a force on the wound to prevent the wound from expanding over time and getting larger. In some implementations, the wound contact layer 102 includes areas that are reinforced to prevent compression, and other areas that are less structurally rigid to allow for compression on the wound bed 114. For example, the wound contact layer 102 can include cushioning to prevent or reduce the occurrence of pressure ulcers or additional wounds. In some implementations, the wound contact layer 102 includes cushioning (or areas that are less structurally rigid) around an outer perimeter of the wound contact layer 102. In some implementations, the wound contact layer 102 includes cushioning in a center of the wound contact layer 102. In some implementations, a depth of the wound contact layer 102 can be altered by compression, changing from a very small depth (e.g., 1-6 mm) to a large depth (e.g., 2-50 mm). In some implementations, a center region of the wound contact layer 102 has a very large depth (e.g., 2-50 mm) and a perimeter edge of the wound contact layer 102 has a very small depth (e.g., 1-6 mm). In some implementations, a depth of at least a portion (e.g., a center portion or an end portion) of the wound contact layer 102 is changeable using a bladder, a material that is expandable when exposed to a stimuli (such as UV light), a cavity in which a chemical reaction occurs that produces a gas or increase in volume of the cavity, or another expansion mechanism. In some implementations, the wound contact layer 102 can be formed from multiple layers that a medical professional can stack on top of each other to obtain a wound dressing 101 of the correct height for a particular wound bed 114. The multiple layers can be attached to one another with adhesive or can move freely relative to adjacent layers.

In some implementations, a thickness (depth) of the wound contact layer 102 is from 1 mm thick to as thick as 10-20 mm.

In some implementations, the dressing 101 is unidirectional or bidirectional (symmetric so there is not top or bottom), and allows for omni-directional flow of fluid, gas and pressure. In some implementations, a unidirectional dressing includes a coating on one side to indicate a directionality to the user (e.g., the use of methylene blue on one side with an instruction to the user to orient the “blue side” up or down). Various other coatings and colors can indicate directionality of the dressing for contacting the wound surface. In some implementations, one or both sides of the dressing incorporates a smooth surface to prevent sheer forces or bumpy surfaces to create uneven pressures to promote tissue adhesion in cases such as skin grafts or collagen applications. In other implementations, a smooth surface of the dressing facing the wound surface may be preferred to prevent sheer forces in the graft. Alternatively, in some implementations, bumps or prominences extending from the surface of the dressing toward the wound surface create spot welds in the graft that allow for improved graft uptake. In some implementations, one or more holes formed in the contact surface of the dressing facing the wound surface enable the dressing to suck tissue up into the holes under pressure creating a spot weld.

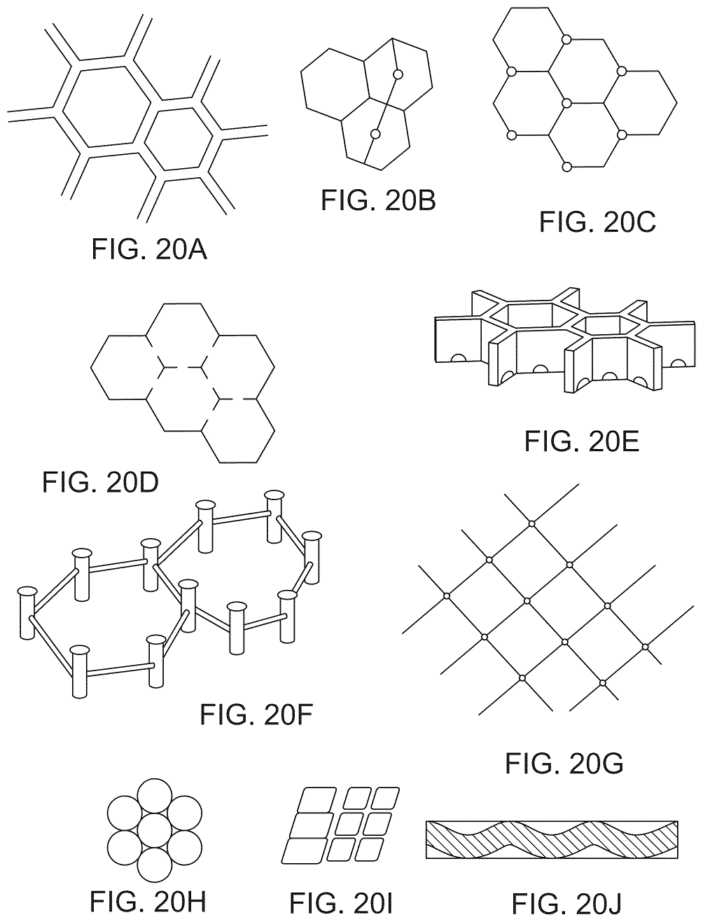

In some implementations, the wound contact layer 102 incorporates a series of holes or protrusions (or bumps), or both. In some implementations, the protrusions are formed as cones with a pointed end facing the wound surface, or as cones with a wide base facing the wound surface. Additional spacing structures are discussed below with regard to FIGS. 20A-J.

In some implementations, the wound contact layer 102 is coated with a coating layer having therapeutic properties. In some implementations, to promote healing, the dressing is coated with proteins, growth factors, medications, elements (silver), chemicals (methylene blue) or other substances in order to promote an environment for healing or cell death in the setting of a cancer or other unwanted tissue (scar tissue, inflammation, heterotopic ossification . . . ). Covalent binding of the dressing 101, and in particular the wound contact layer 102, with multiple drugs, ions, chemicals, biologics, synthetic proteins, synthetic materials or elements allow for improved wound-specific healing. The bonding between the therapeutic substances and the dressing 101 or wound contact layer 102 may be permanent, or, alternatively, are designed to erode or dissolve over time to release therapeutics from the wound contact layer 102 over a prespecified time frame as the needs of the wound change. For example, anti-bacterial coatings might be permanently bonded to the wound contact layer 102. As another example, chemicals to promote vascularization, epithelialization, granulation promotion or to prevent biofilm development on the surface of the dressing 101 would be permanently bonded to the wound contact layer 102. These permanent coatings support a prolonged use of the wound dressing 101 (i.e., 7 or more days). Non-permanently bonded coatings can include coatings designed to elute particles, ions, chemicals, proteins, elements over time as the wound contact layer 102 is exposed to bodily fluids, enzymes, or reagents.

In some implementations, one or more coatings can be applied only to the wound contact layer 102 to assist medical practitioners in determining which side of the contact layer 102 should be positioned facing against the wound. In other implementations, one or more coatings can be applied to the entire dressing 101 or to at least a portion of the dressing 101 (e.g., a portion that does include, or does not include, the wound contact layer 102). Coatings can be added to the dressing 101 to increase effectiveness against bacteria and other contaminants, and/or to otherwise make the dressing 101 more effective in healing wounds.

In some implementations, the wound contact layer 102 includes multiple layers of therapeutic coatings to create an intentional time-dependent profile of therapy provided to the wound bed 114 to match a progression of wound healing, and to interact with bioactive molecules, chemicals, and other therapeutics. For example, in one implementation a permanent coating can be bonded to the wound contact layer 102, followed by one or more coating layers that are temporarily bonded and designed to elute into the wound over time. The temporarily bonded layers could elute or erode over time releasing a multitude or chemicals, therapeutics, ions, elements, gases, or fluids. In some implementations, the release of therapeutics is controlled by a layering technique, or by relying on a known rate of breakdown of a substance or coating layer. Bonding therapeutic substances to a coating of the wound contact layer 102 can be accomplished by a variety of techniques, including UV light, chemical bonding, or heat bonding. Additionally, soaking, baking, or acidic reactions can be used to attach coatings to the wound contact layer 102. A multitude of energy sources or chemical reactions or fusions can be utilized to permanently or temporarily bond coatings or substrates to the wound contact layer 102 for delivery of therapeutics. These substrates can be elements, ions, gases, specific enzymes, medications, or chemotherapeutics. A reaction can occur at the wound bed 114 based on a solid or gas or liquid either contained in the wound contact layer 102 of the dressing or supplied to the surface of the wound bed 114 through the dressing 101.

In some implementations, therapeutics having bactericidal or antimicrobial (e.g., fungus, virus, other microbes) effects can be delivered to the wound bed 114 through the wound dressing 101 or can be incorporated into the wound dressing 101. For example, such therapeutics can include betadine, chlorhexidine, hydrogen peroxide, Dakins solutions, or other suitable bactericidal therapeutic compounds. In some implementations, the dressing 101 can include a coating or integrated therapeutic that is intended to induce specific or general cell death. Specific tissues can be targeted by selecting a particular therapeutic for the coating that targets specific tissues or cells. Alternatively, a therapeutic can be applied via a coating that has a general toxic or poisonous effect. The toxic or poisonous effect can be selective for certain foreign cells (i.e., microbicidal) or for all cell types (e.g., to reduce hypertrophic granulation) or for targeted cells. For example, a therapeutic having a toxic effect on particular targeted cells may be used when the dressing 101 is placed in proximity to a cancerous lesion or resection bed following removal of a cancerous lesion. The coating could be directed specifically to the cancer cell (i.e., directed to rapid dividing tissue or immunotherapy-based targeted therapy) by targeting tissue specific characteristics. For example, the tissue can be targeted based on the type of organ tissue or specific physiological characteristics of the tissues or cells. As described above, the timing can be controlled as well as the duration of the release.

In addition to application of therapeutics through coatings of the wound contact layer 102, chemical reactions or therapeutics can be directly applied to the wound bed 114 through prefabricated channels, pathways, tubes, and flow paths of the wound contact layer 102. In some implementations, therapeutics are delivered through the inlet tubing 110 and suction dome assembly 109 to the wound bed 114 through the large-diameter passageways of the wound contact layer 102. The therapeutics can be delivered through the inlet tubing 110 to the dressing 101 at specific times or intervals by administration by a medical professional or automatically using the EVR to control dosage and timing. In some implementations, suction dome assembly 109 is replaced by a vacuum interface.

In some implementations, therapeutics are introduced through the dressing 101 through separate inflows/outflows of the dressing 101 (not shown) or through the large-diameter passageways of the wound contact layer 102. In some implementations, the wound contact layer 102 includes contiguous large-diameter passageways dedicated to distribution of therapeutics to the wound bed 114. In some implementations, the large-diameter passageways of the wound contact layer 102 allow for random diffusion across the wound. In some implementations, the dressing 101 further allows for distribution of therapeutics to the periwound surrounding the wound. In some implementations, therapeutics distributed to the wound bed 114 include gases, liquids, gels, solids, powders or a mixture of some or all. In some implementations, therapeutics distributed to the wound bed 114 are intended to fight or prevent infection; increase blood flow or healing; prevent formation of chemicals or growth factors; provide needed proteins, nutrients or chemicals to the wound bed 114; reduce inflammation; increase inflammation; promote or inhibit apoptosis or other chemical pathways such as hemostasis; or are intended for various other purposes. In some implementations, therapeutic agents provided to the wound bed 114 are designed to be visualized through direct visualization, special sensing devices such as black light, fluorescence, near infrared spectroscopy, other forms of spectroscopy, heat, cold, release of gases or other chemicals.

In some implementations, in addition to (or alternatively to) a coating providing therapeutic substances to the wound bed 114, the wound contact layer 102 includes a coating or surface designed to allow a metered, intentional, and controlled depth of tissue debridement or ingrowth to achieve an effect on the tissue of the wound bed 114 similar to wet to dry dressing changes. The wound-contacting surface of the wound contact layer 102 can be designed to be smooth, rough, perforated or impermeable, and can be non-porous or porous. The rough surface allows for wound cleaning or debridement of the surface tissue layer of the wound bed. The rough surface can further allow for minimal or controlled tissue ingrowth to allow for devitalized tissue removal. In combination with the surface features of the wound contact layer 102, gases can be provided through the inlet tubing 110 to the wound bed 114 to dry the wound surface to accelerate necrotic tissue removal. Enzymes can also be provided through the inlet tubing 110 or in the wound contact layer 102 to remove biological material to create a clean wound bed 114.

In some implementations, UV light can be used to reduce bacterial overgrowth om the wound bed 114, which can be detrimental to wound healing and lead to infection. The wound contact layer 102 can be formed from a polymer which is permeable to UV light (i.e. clear/translucent) so that UV light can pass through the wound contact layer 102 to a surface of the wound bed 114 underlying the dressing 101. In some implementations, a UV light source can be built into the dressing 101 to provide antimicrobial protections to the wound bed 114. In some implementations, LED UV lights, visible lights (e.g., red, blue, or the entire color spectrum) other sources of UV light, or other means of energy are transmitted through the dressing in order to disinfect the wound. The lights can be created in series or in parallel to allow the periphery of the dressing 101 to be trimmed or cut away, while still allowing the lights to function within the dressing 101 or on a surface of the dressing. The wiring can be associated with the tubing 110, 112 and the wiring can expand out from a central area of the dressing 101 to provide continued coverage as the edges of the dressing 101 are trimmed. In other implementations, a UV light source external to the dressing 101 is used to provide UV light to the wound bed through the dressing 101. For example, a UV light source can be attached to the suction dome assembly 109 and propagated through the dressing 101 through the large-diameter passageways. UV-C light is a recognized safe and effective means for reducing bacterial growth on living tissue, as well as on surgical implants. In some implementations, a UV-C light source is contained in the wound dressing 101. In other implementations, the wound dressing 101 is formed to allow the UV-C light to penetrate through the dressing 101 to the wound bed 114, for example where the wound contact layer 102 is formed from a transparent material or where the passageways of the wound contact layer 102 are formed to direct UV-C light therethrough. Reflective properties can be mixed into the polymer forming the wound contact layer 102 in order to amplify the UV light so that it is passed through the wound contact layer 102 to cover the entire surface of the wound bed 114 despite limited light sources.

In some implementations, alternative means for sterilization of the wound can be used either to sterilized the wound surface or fluid to irrigate the wound. Any means of energy can be used or chemicals to sterilize the wound or local non sterile fluids in order to wash the wound. Dielectrode barrier discharge elements can be used for cold plasma treated fluids. This can be used in military uses or mass casualties situations where supplies are limited and scarce.

In some implementations, the dressing can also be used to deliver sustained chemical or medical therapy to a desired area of the body over an extended period of time. The dressing and its flow pathways for exudate removal can be utilized to deliver medications, chemicals, gases, liquids, or other materials to a dedicated area. This mechanism can be used to deliver antimicrobial or anti-biofilm chemicals to metal or plastic or other foreign material in the form of implants within the body such as joint arthroplasties or other vital implants. The medications can be delivered continuously or in bolus fashion. Chemicals can be administered and then deactivated with other chemicals or washed out using flushes. Flow can be through multiple domes or a single dome with both inflow and outflow pathways. These processes can be used for cancer treatment, infection, dialysis, or other chemical processes in humans or animals (veterinary) medicine.

Monitoring of the wound health can be performed within the dressing or away from the dressing. For example, in some implementations, the wound (e.g., exudates from the wound) can be sampled through the dressing/outflow tubing or other means. The dressing 101 can allow for a separate third or fourth tubing that samples the wound at a single location or at multiple locations. That sampling tubing can be coupled to a luer lock where a syringe is used to collect the wound exudate for sampling. This separate tubing can prevent contamination from the dressing or other issues.

In some implementations, monitoring with or without power needs can be performed within the collection canister or just prior to the collection canister within the tubing. In some implementations, the sensors, monitors or indicators are positioned within the tubing near the dressing as described above. In other implementations, the sensors, monitors or indicators are positioned in the tubing or on the canister prior to the collection area. If the sensors are at the collection canister entrance, a power source from the pump facilitates monitoring, recording, tracking, and reporting/alarming based on the monitored parameters. In some implementations, the monitors sample exudate or irrigant after it has passed over top of the wound. Since the dressing does not retain fluid, a far smaller bacterial load will be present to confound the results of wound sampling compared the conventional sponge dressings which retain fluid and promotes bacterial overgrowth.

Direct sampling of the wound surface without requiring the removal of the dressing is also possible. For example, fluorescent proteins that isolate bacteria or other special biologics or chemicals or proteins can be used to coat the wound prior to placement of the dressing or while the dressing is in place through the irrigation supplied through the tubing. In such cases, black lights or other detection means can be used through the dressing to determine specific characteristics of the wound such as bacteria, growth factors, proteins, or other chemicals or gases. In some implementations, the dressing includes electrical and/or chemical monitors. In some implementations, the monitors are designed to function with a battery, without power, using solar power, or using wireless power transfer. In some implementations, chemical monitors include chemical indicators that are either reversible or irreversible can be created for things such as Ph, temperature, specific chemicals or proteins or growth factors. In some implementations, indicators are positioned throughout the dressing. In some implementations, indicators are located at the center of the dressing. In some implementations, indicators are located within the dressing. In some implementations, indicators are located external to the dressing (e.g., on an external surface of the dressing). In some implementations, a position of the electrical and/or chemical monitors in the dressing is customizable. The monitors can communicate with the EVR 107 through wired or wireless communication channels.

In some implementations, the dressing 101 is designed to be transparent, translucent, or clear to allow for wound monitoring while the dressing is in place. Wound monitoring can be performed using fluorescence and a black light utilizing bacteria's natural fluorescence. Visible and/or non-visible light can pass through the dressing to initiate a chemical reaction (for example, activating methylene blue to create oxidized oxygen radicals such as singlets which are used to fight foreign microbes and rapidly-dividing cancerous cells). In some implementations, oxygen, visible light, or another initiating reagent are provided to the wound bed 114 through the dressing using the large-diameter passageways to initiate treatment of the wound bed 114 under the dressing. In some implementations, modifying the coating on the dressing 101, the supply of oxygen, light exposure, and/or specific wavelengths can increase or decrease the therapeutic effects of the chemical reaction(s) initiated from the dressing 101. In some implementations, supplemental oxygen can be supplied to the wound bed 114 to increase therapeutic effectiveness in cases of contaminated wounds or infections. In some implementations, nebulized or dry oxygen, or other gases or liquids, can be used to increase therapeutic effectiveness. In some implementations, temperature can be modified to increase or decrease therapeutic effectiveness using the chemical reactions.

In some implementations, supplemental visible light or specific wavelengths can be applied to the wound bed 114 through the addition of one or more light-emitting diodes (LEDs) with the dressing 101. In some implementations, supplemental visible light or specific wavelengths can be applied to the wound bed 114 through application by an external source (e.g., an external source configured to provide supplemental visible light or specific wavelengths). In some implementations, the duration, pulse, or exposure pattern of the supplemental visible light or specific wavelengths can be modified to increase the effectiveness of the chemical reactions applied to the wound bed 114. In some implementations, dilutants or solvents can be passed through the dressing 101 to manipulate therapeutic efficacy. The dilutants or solvents can also be used to irrigate the wound bed 114 in addition to their therapeutic effects. In some implementations, additional materials, chemicals, or solvents can be covalently or ionically bonded, or bonded by other mechanisms, to the dressing 101.

In some implementations, where the dressing 101 is not fully transparent, a selective coating can be applied to allow for visualization of a portion of the wound. For example, ‘windowing’ can allow for visualization of the wound if a coating is not translucent. In some implementations, windowing or striping can be used to control an amount of chemical release or dosing of a material onto the wound surface. In some implementations, striping of two or more chemicals in a coating can be used to create a chemical reaction that would promote a therapeutic reaction.

In some implementations, the dressing 101 includes one or more selectively permeable barriers or sealing layers. In some implementations, a selectively permeable barrier promotes the transmission of certain gasses into or out of the dressing 101. For example, a selectively permeable sealing layer may allow oxygen to pass through into the dressing 101, but may prevent other, less desirable, gases from entering or exiting the dressing.

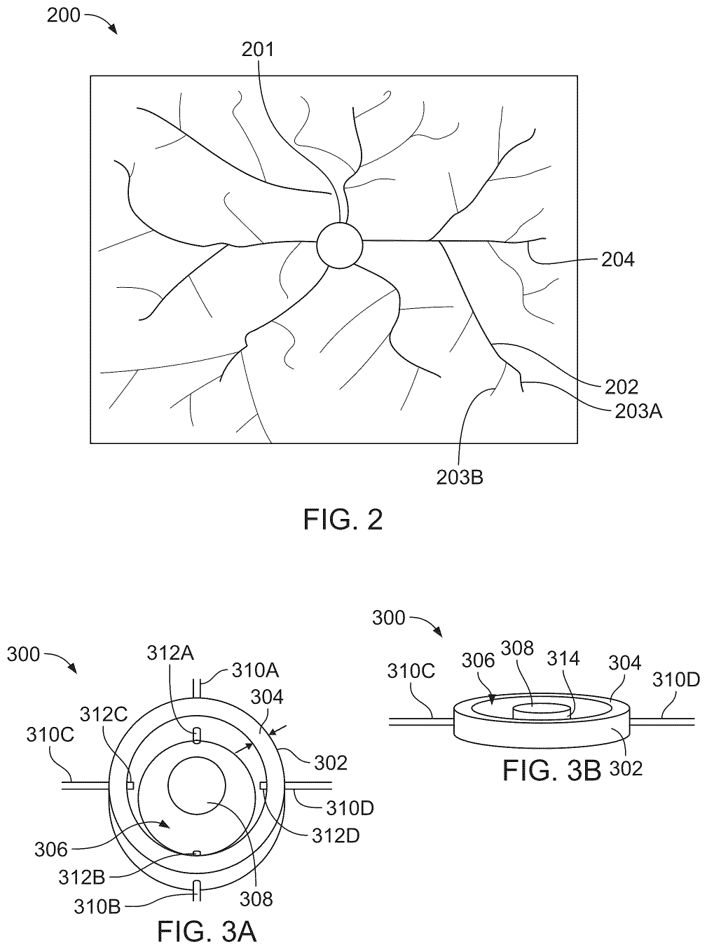

In some implementations, the wound contact layer 102 includes wiring extending throughout at least a portion of the wound contact layer 102. FIG. 2 illustrates a plan view of a section of a dressing 200 including wiring distributed throughout the wound contact layer 102. The wiring is formed as a radial network extending from a central core 201. From the central core 201, radial arms 204 extend out toward the edges of the dressing 200. Branched arms 202 extend from the radial arm 204 and secondary branched wirings 203A-B extend from the branched arms 202. The wiring pattern of the dressing 200 covers the area of the dressing without any “dead” spaces where wiring does not extend. The wiring pattern is built into the polymer forming the dressing so that the wiring can be cut or trimmed with the dressing to fit the wound contact surface without disrupting the function of the wiring. In other words, the edges of the dressing 200 can be cut, so that portions of the radial arms 204, branched arms 202 and secondary branched wirings 203 A-B can be cut through and detached from the central core 201 while enabling wiring circuits to function properly.

The wiring through the dressing can be used to provide power to any number of electrical devices incorporated into the dressing (for example, dressing 101 in FIG. 1). In some implementations, the wiring can be used to provide power for an electromagnetic pulse for bone healing, UV light for bio-reduction, lighting to assist in visual inspection of the wound, Near Infrared Spectroscopy (NIRS) for determination of perfusion and blood supply, and pressure measurements for monitoring to ensure negative pressure is distributed throughout the dressing. In some implementations, the wiring can be used to provide heating or cooling to the wound bed. In some implementations, the wiring is coupled to one or more sensors in the dressing 101. Wired sensors can be constructed in series (to allow for trimming of the dressing 101 to customize the shape or size) or in parallel. In some implementations, sectional wound monitoring is performed using wired sensors.

In some implementations, NIRS or another suitable method is used to measure perfusion of blood at the wound surface. In some implementations, NIRS or another suitable method for measuring perfusion is used to guide or automatically adjust a negative pressure setting of the EVR 107. In some implementations, the NIRS sensor provides feedback to the pump at the EVR 107 to dial in a negative pressure values suited to provide optimal perfusion of the particular wound and to maximize blood flow under the dressing 101. In some implementations, chemicals or gases (e.g., NO) can be introduced to the wound bed in response to perfusion measurements by the NIRS sensor and may be automatically adjusted based on the NIRS sensor measurements or on other indications of blood flow. In some implementations, the dressing 101 further promotes increased blood flow to the wound under the dressing 101 by the inclusion of a heating coil into the dressing 101 near the wound surface.

In some implementations, thermal cautery is integrated into the dressing 101 to provide hemostasis. For example, in some implementations, the dressing 101 includes one or more coatings of chemicals to initiate a chemical reaction within the dressing. In some implementations, the coatings of chemicals react to blood, fluid, sunlight, UV light, or another initiating reagent to create a chemical burn in order to obtain hemostasis in an emergent setting. In some implementations, wiring within the dressing (as discussed above) are activated to provide a quick burst of heat to cauterize the wound without thermal damage or pain. In some implementations, therapeutics including local anesthetics are provided through the dressing to the wound bed 114 prior to cauterizing.

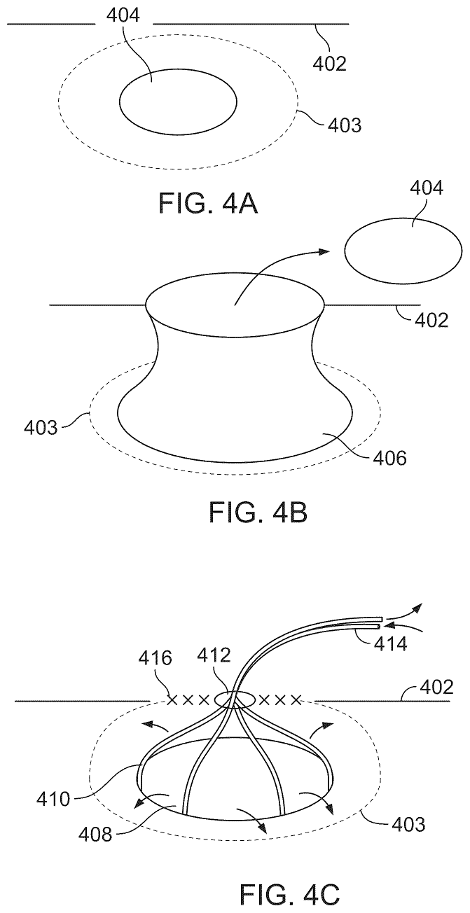

In some implementations, the wound dressing 101 includes mechanisms for reducing ambient humidity at the wound bed 114 or gross moisture on the surface of the wound bed 114. For example, in some implementations, the wound dressing 101 includes tubing or additional passageways to allow air flow through the dressing 101 or to allow other chemicals/gases that dry the exudate or skin through the dressing 101 and to the wound bed 114. In some implementations, filters and flow restrictors can be utilized with the additional passageways or tubing to prevent leak warnings and still maintain the desired negative pressure levels at the wound bed 114. The negative pressure can allow suction of gases over the wound or, in the case of a closed wound, allows gases to be transmitted over sutures or staples or other closure mechanisms. In some implementations, a filter system allows for cleaning of the air or gases to reduce moisture before the gas flows over the wound bed 114. In some implementations, additional agents (hyper absorbent dust/powder) are introduced and evacuated through the system. For example, in some implementations, alcohol or other drying agents or gases are passed through the system. For example, when the wound dressing 101 is used with an incisional or closed wound, reducing the moisture at the wound surface is important to avoid maceration. The skin is normally a dry structure, and adverse events, like maceration, fungal or bacterial overgrowth, can occur when it is kept moistened for prolonged periods. For example, layers of porous or absorbent material can be integrated into the dressing with mechanisms for preventing the ingrowth of tissue as the wound heals and closes. Wound dressings for use in incisional wounds are described further below with regard to FIGS. 4-12. Additionally, ambient humidity in the wound bed 114 can be reduced by using the EVR 107 to control suction through the dressing 101 and at the wound bed 114 to remove exudate or moisture. In some implementations, the EVR 107 or another control device is used to control a supply of gas to the wound bed 114 to dry the wound bed 114.

In some implementations, the dressing 101 also includes one or more catheters or expandable balloons to provide constant or intermittent positive pressure for hemostasis. The EVR 107 is used to monitor and regulate the amount and duration of pressure provided to the wound by the catheter(s) or balloon(s). In some implementations, a mechanical pump can be used (like a blood pressure cuff) to inflate or deflate the bladder/sack. In some implementations, the bladder(s), balloon(s) or catheter(s) are coated with chemicals to promote hemostasis.

### II. Electronic Vacuum Regulator (EVR)

The EVR 107 regulates and controls the flow through the wound dressing 101. The flow can be electronically or mechanically regulated. Additionally, in some implementations, the EVR 107 controls the type of gas provided to the wound bed 114, a mixture of gas provided to the wound bed 114, the rate at which the gas is provided to the wound bed 114, the concentration of the gas, the humidity of the gas, the temperature of the gas, and the pressure of gas (positive or negative pressure), and the duration over which the gas is provided to the wound bed 114 to obtain the desired effects on the patient. In some implementations, a rate of oscillation between any of these conditions is controlled by the EVR 107. Inert gases or reactive gases can be used in treatment of the wound bed 114.

In some implementations, the EVR 107 provides a mechanical motion of all or part of the wound dressing 101. Mechanical motion can be used to debride the surface of the wound bed 114, for example if a wound contact layer 102 of the wound dressing 101 has a roughened wound-facing surface. Automated systems can be used in combination with the wound dressing 101 such as repeated pneumatic inflow or bladders. Vibration through sound, fluid infusion or suction can also be utilized to provide motion of the wound dressing 101 in support of debridement. Alternatively, in some implementations, manual manipulation of the wound dressing is used to achieve debriding of the wound surface.

In some implementations, the EVR 107 provides control for an external suction source (e.g., wall suction or a separate pump). In other implementations, the EVR 107 can include pump machinery so that the EVR 107 can be used without an additional suction source. This allows for the continued use of the EVR 107 to provide suction to the wound in situations where additional suction is not available (e.g., during transport within or outside of a hospital, used by first responders to accidents/incidents, used by medical personnel in military situations).

For an EVR 107 including pump machinery, the EVR 107 can be designed for customizability to a particular patient and situation and for enhanced interaction by medical professionals to simplify medical processes surrounding wound care. The EVR 107 can be designed to have a removable smart portion (sometimes referred to as “IQ” herein) similar to a stereo face in car stereos that allows for the device to be separable into a programable part and a separate part which carries the larger pump machinery (sometimes referred to as headquarters or “HQ” herein). The HQ can be designed to have multiple sizes or strengths. Since, when used with the dressing 101 of FIG. 1, the wound contact layer 102 is a polymer and not a sponge, the power or suction force required to create a negative gradient is much lower than required for dressings including sponges, because sucking through a wet or saturated sponge requires a large force. Additionally, the large-diameter passageways through the wound contact layer 102 can further reduce the suction force and power required of the EVR 107, as described above.

In some implementations, the EVR 107 includes a tracking system to provide accurate location of the EVR 107 or other components of the system, such as the collection cannister 106 or wound dressing 101. Retrieving pumps from a patient at the end of care can be a very challenging problem in the out-patient setting. Being able to locate the devices using a tracking system assist in pump retrieval because the location of the pump can be ascertained and documented. In some implementations, the EVR 107 uses GPS or another location identification system to allow for identification of a location of the EVR on a map and/pr within a building or system such as a hospital or business campus. In some implementations, the EVR 107 provides altitude information to determine location based on which floor in a building a unit is located in. In some implementations, the EVR 107 includes a back-up battery that is automatically charged when plugged in. The back-up battery can provide the tracking system with an updated location to allow for accurate location similar to current tracking devices such as an Apple AirTag or Tile. The back-up battery can signal location even with a dead main battery. In some implementations, the EVR 107 includes a signaling beacon or identification chirp to identify the location within a room. Additionally, in some implementations, the EVR 107 can provide a location indicating beep, noise, or other alarm to assist in locating the device. The location indicating noise can be triggered by an external device (e.g., a computer can be used to trigger the noises) or the noises can be set up to be triggered by the presence of a device, such as a scanner for scanning a barcode of the EVR 107 or a mobile device running an application for locating the EVR 107.

In some implementations, signals from the EVR 107 are sent to a specific central location monitoring system maintained by the manufacturer which can assist is determining the last recorded location of the device prior to battery depletion. Once activated, a new location can be determined similar to cell phones. This location monitoring can be centrally at the manufacturing site or established through an app or computer program that allows providers to monitor the location of its multiple units. As batteries start to run low, alarms can be signaled to locate and recharge the units. Monitoring for owners or distributors can be established for provider owned units similar to find my phone apps

In some implementations, two-way communication between the EVR 107 and any type of monitoring system or clinical provider can be performed. This can be especially useful in an outpatient setting to allow for a medical professional to communicate to a patient. For example, the EVR 107 can display messages from the provider on a display screen of the EVR 107 if the unit has not been used or turned on within a predetermined period of time to assist with patient compliance with a therapeutic treatment. In some implementations, the EVR 107 can be remotely locked or shut down if the required therapy for which the EVR 107 was being used has been completed, and the EVR 107 has not been returned in a predetermined period. In some implementations, the EVR 107 can provide warnings to a patient or provider that indicate a need to replace suction cannisters. In some implementations, the EVR 107 can be used to provide desired or ordered therapeutics such as irrigation or oxygen. Additionally, in some implementations, messages containing reminders for upcoming clinic visits can be sent across the screen of the IQ. The EVR 107 can be connected to the internet via Bluetooth, satellite or other means including wired and wireless connections.

In some implementations, two-way communication occurs between the wound dressing 101 and the collection canister 106 containing exudates removed from the wound or the EVR 107 to confirm wound healing. For example, the collection canister 106 can include testing mechanisms to test the exudate for bacteria or other signs of infection. If bacterial DNA is sampled in the exudate, the EVR 107 receives this test result and issues a notification to the clinical provider. For example, at initiation of care, a provider can indicate email or another preferred communication method for receiving such alerts. The EVR 107 may additionally issue reminders based on feedback from the testing of the collection canister 106 and the emails or alerts can be issued via email, automated texts to the display of the EVR 107 or external pump unit, or cell phone.

In some implementations, the EVR 107 receives instruction via an application (an “app”) run on a mobile device or computer. An interactive app allows for instructions to be transmitted to the EVR 107, and for the EVR 107 to respond with communication by the HQ confirming that the instructions were correctly followed and that therapy was administered at a particular time. Additionally, the interactive application provides a record of treatment including time stamps and documentation. In some implementations, troubleshooting or updates to the EVR 107 are accomplished through the app. The app can be used to upload data via wired or non-wired means, including via Bluetooth or other means of communication.

In some implementations, additional components of the wound dressing system can be tracked using a microchip or other means, including radiofrequency (RFID), bar codes, QS or QR codes. For example, wound dressing 101 can include an identifier 120, such as a barcode, microchip, QR code, label, or other suitable identification means. Microchips, RFID codes, bar codes and QR codes can be used to track not only the wound dressing 101, but to also identify the patient, pump/EVR 107, therapeutic cartridges 117, and the collection canister 106. A barcode reader, laser scanner, optical scanner, or mobile device can be used to track and log the wound dressing 101, to keep records related to the patient and the treatments received. In some implementations, in addition to wound dressing 101, any therapeutic cartridges 117 containing therapeutic compounds or gasses for administration through the wound dressing 101 (e.g., disposable therapeutic ampules or gas containers) can be equipped with an identifier 121 such as a microchip that allows for communication with the EVR 107. For example, the therapeutic cartridges 117 can be sized to be inserted into a docking port 119 of the EVR 107 (similar to printer cartridge) and the identifier 121 is read by the EVR 107 to confirm that the therapeutic contained in the therapeutic cartridge 117 is appropriate (e.g., similar to a printer cartridge chip that confirms accurate manufacturer and toner levels). These identifiers 121 of the therapeutic cartridges 117 can communicate with the EVR 107 to ensure that the correct therapeutic compound or treatment is being administered to the patient. Additionally, the identifiers 121 of the therapeutic cartridges 117 can also communicate with the clinical provider that the therapeutic was in fact administered in a home or other out-patient environment, that the therapeutic treatment was completed, and when the therapeutic treatment was actually administered. Therapeutic cartridges 117 can be used for multiple treatment sessions, and the EVR 107 can provide a warning when capacity of the cartridge is low, prompting a user to reorder or to communicate with the provider. The EVR 107 can be in communication with electronic patient records to medical record data, patient identification data, material identification data, and treatment data into the record.

In some implementations, these therapeutic cartridges 117 can be powder ampules, liquid ampules, or gas canisters. In some implementations, the therapeutic cartridges 117 require one or more of mixing, heating, cooling, UV light activation. In some implementations, no mixing is required. In some implementations, the therapeutic cartridges 117 require a particular activation sequence to prolong storage of the therapeutic compounds in the cartridge. The activation sequence (for example, mixing of liquids or gasses, heating/cooling or providing UV or visible light) can be performed within the vacuum or outside of it. In some implementations, the activation sequence may be initiated by puncturing a seal of the therapeutic cartridge 117 (within the EVR 107, during insertion into the EVR 107, or prior to insertion). In some implementations, the puncturing of the seal of the therapeutic cartridge 117 allows for air to initiate the activation sequence or punctures a fluid sack for mixing with a powder to initiate the activation sequence. In some implementations, the therapeutic cartridge 117 can be activated by requiring an ampule to be broken through squeezing or bending the therapeutic cartridge 117 (for example a vial). In some implementations, the therapeutic compound from the therapeutic cartridge 117 is passed through a filter prior to application or dispensing in the wound dressing 101. In some implementations, the EVR 107 includes a screw-in mechanism in the therapeutic docking cavity 119 allowing for the therapeutic cartridge 117 (for example and ampule) to be inserted into a correct position in the docking cavity 119 and puncturing a seal of the therapeutic cartridge 117 for activation of the therapeutic compound. In some implementations, gravity can be used to release the therapeutic compound from the therapeutic cartridge 117 to the system. Alternatively, the EVR 107 can engage a pump or provide suction to pull the therapeutic compound out of a well-type system within the therapeutic cartridge 117 to control an amount of therapeutic compound provided to the wound bed 114.

The therapeutic cartridge 117 can be color-coded to allow accurate selection of the appropriate cartridge by patients. The therapeutic cartridge 117 can be sized and shaped so that it fits only in certain docking cavity 119 on particular EVRs 107. For example, gas-based therapeutic cartridges 117 can be shaped differently from liquid or powder-based therapeutic cartridges 117 to ensure that they are positioned in the correct docking location 119 of an EVR 107. As another example, therapeutic cartridges 117 that contain medications can be sized or shaped differently than therapeutic cartridges 117 that do not include medications, so that the cartridges containing medications can only be used with particular EVR 107 types or particular docking locations 119. Additionally, color-coding the therapeutic cartridges 117 not only helps to ensure the correct therapy is administered by medical professionals, but can also assist patients who may be illiterate or who speak or read other languages to select the correct therapeutic cartridge. The EVR 107 can display drawings and instructions on a screen to assist in accurate therapeutic delivery.

In some implementations, the EVR 107 includes a system for monitoring the wound bed and/or physiological parameters of the patient, in addition to status and parameters of the dressing, EVR, pump, and collection canister. For example, a monitoring system can be designed and incorporated into the system that provides manometer readings (negative pressure readings) at the wound surface or at other locations in the dressing and/or system. In other implementations, the dressing includes contact pressure sensors which transmit a measurement to the EVR 107 to monitor wound conditions. Other sensors and devices can be used to monitor wound conditions, including blood flow at a superficial level, deep in the wound bed, and/or at the periwound.

In some implementations, the EVR 107 includes software to automate monitoring of wound surface healing and the presence of infection or bacteria. In some implementations, the EVR 107 further includes software to automate therapies based on present algorithms for treatment of the wound, which may be individualized and preset by a physician. In other implementations, the EVR 107 further includes software to automate therapies based on present algorithms to distribute medications and therapeutics to the patient if certain conditions or set of conditions are recognized by the sensors. In some implementations, the EVR 107 includes software to provide a recommendation to a physician based on sensed and monitored conditions at the wound bed Recommendations can include changing the dressing, adjusting a setting, administering a therapeutic or medication to the wound surface, administering a therapeutic or medication to the patient via another means (e.g., oral or IV medications). For example, IV fluids can be prompted or recommended to be initiated in situations where conditions sensed by the EVR 107 indicate concerns for low blood pressure or exsanguination. In some implementations, the EVR 107 can incorporate other vital sign monitors in addition to dressing monitors/sensors. IV access and medication delivery can be pre-set and administered to the patient by a medical professional based on wound and vital sign readings collected at the EVR 107. In some implementations, wired or wireless communication can be performed between the dressing, the pump and other potential devices such as a wound tensioner, hemostatic device, or other suitable device. Additionally or alternatively, in some implementations, the EVR 107 includes mechanisms for providing wired or wireless communication channels between the pump, the electronic medical record, hospital alarms, hospital paging systems, clinician cell phones, on-call doctors, patient cell phones, patient's emergency contact number, or other appropriate notification systems.

In some implementations, pain control can be administered through the EVR 107. Local anesthetics can be used to limit the need for narcotics or systemic pain medication. Correct dosage of local anesthetics can be determined by the EVR 107 based on patient characteristics such as age, BMI, height, weight, sex or other demographics. In some implementations, the EVR 107 includes locks similar to a patient-controlled analgesic machine to limit over-administration. In some implementations, the EVR 107 allows the patient or provider to control the delivery of pain reducing agents (such as morphine or local anesthetics) through the vascular bed/granulation bed of the wound bed 114. For example, NSAID's, Tylenol, narcotics, anti-anxiety, gabapentin, local anesthetics, capsaicin and other pain modifying or relieving medications could be utilized directly on the injured/healing area to reduce amounts of pain relievers required to provide relief and to reduce the systemic effects of the pain medications. These medications can be provided in a fluid/liquid form or as a gas or nebulized form to prevent drying out the wound bed 114. A mixture of multiple types (liquid/fluid/gas) can be used to reduce total amounts of each type of medication.

Although EVR 107 is depicted in FIG. 1 with wound dressing 101, EVR 107, including any or all of the features described above, is compatible with the other dressings described below, including dressings illustrated and described with regard to FIGS. 4-19 and 21-22. Various embodiments are configured to include an advanced electronic vacuum regulator (EVR), for example, the EVR 107 of FIG. 1. This device measures flow, output and pressure at the canister and receives, interprets and responds to these monitors as well as monitors positioned in or on the wound dressing. The enhanced monitoring ensures reliable and consistent therapy that can be safely applied for longer periods of time.

In some implementations, an intermediate vacuum source is used. Typical vacuums for NPWT require a large powerful pump because when negative pressure is placed on a sponge, the sponge collapses and especially when saturated the flow pathway is restricted. In the dressings described here, the flow pathways resist compression, and accordingly reduce flow resistance. In some implementations, a polymer semi-rigid wound contact layer can reduce the power needs of the pump because of improved flow characteristics and reduced resistance. Reduced resistance allows for the dressing to be used with less powerful vacuums. In some implementations, a battery-powered vacuum is used with the dressing. In some implementations, the pump may need to be larger than the current disposable pumps (e.g., larger than a “pico system” pump). However, a reduced size pump relying on AA or AAA batteries can be utilized with a dressing to provide NPWT. In some implementations, a pump using a rechargeable lithium battery can be used. In some implementations, the fluid collection canister can be converted to a collection bag or one that can be emptied and reused. The bag can be filled hyper absorbent materials which can be added once the canister is emptied. In some implementations, the collection bag can be a liner inside a hard plastic or other material collection cannister. The liner may be disposable, but the canister can be cleaned and reused with the same patient or between different patients. Replacing only the liner reduces waste, manufacturing costs, storage space, and ultimately saves valuable resources.

In some implementations, pumps can be equipped to collect payment such as typical credit card scanners. Insurance cards can be scanned and sent to a billing agency from a scanner on the pump. In some implementations, the pumps can be remotely controlled so as to power down the pump if they are not returned, preventing the pump from being used without permission or approval. Activation codes can be obtained through use of different payment codes in order to activate a pump that has been deactivated after a certain period of time, or when control over the pump has been lost. In some implementations, a tracking device can be utilized to determine the location of the pump (similar to tag-based tracking systems or tracking of electric scooter locations in large cities). In some implementations, the pumps can be located and serviced or unlocked or locked remotely. In some implementations, the pumps can be tracked with apps or sound alarms when close by in order to locate the pumps. In some implementations, a separate battery solely for location alarms can be used in order to prevent dead batteries from delaying or preventing the pump from being located.

### III. Collection Canister

Collection canister 106 collects exudates suctioned from the wound bed 114 by the EVR 107. Conventional collection canisters require gravity to separate air from fluid: the air/fluid mixture is pulled across a suction canister or is deposited into a canister from a location towards or at the top of the canister and is directed downward. In such conventional systems, the suction outflow from the canister is also located toward a top of the canister. Therefore, in these conventional systems, flow of the exudate and other liquids travels from the top of the canister to the bottom, so that the liquid is left at the bottom while the dry air is suctioned out the top. In collection canister 106, directional flow is used instead of gravity to separate air from liquids. The directional flow can be directed around curves or through channels around hyper-absorbent material to dry the air/gas in the canister. Because the liquids/air are separated by directional flow techniques rather than by gravity, collection canister 106 can be formed from a flexible bag rather than a hard canister. A flexible bag for use as a collection canister 106 can be foldable, pliable, light-weight, easy to pack, and disposable to save on storage and cost. In some implementations, the flexible bag includes a screw-on lid opening and is designed to fit within another suction canister, and further includes a magnetic or other connection between the bag and the interior of the suction canister to hold the bag open without collapsing with the application of negative pressure/suction. In the flexible bag, the directional flow techniques can include directing the fluid/gas through U-turns or tortuous pathways of hyper-absorbent material. By changing flow direction and/or speed of the air/gas and liquid, the absorbent material is able to dry out the air/gas so that the air can be cooled to allow condensation of the fluid out of the mixture. For example, the air/gas can be passed over a multitude of right angles formed from an absorbent material to allow the air/gas to condense and to utilize gravitational forces to aid in condensation regardless of the alignment of the collection canister 106 (or flexible bag forming a collection canister 106).

In some implementations, the wound dressing 101, or specifically, wound contact layer 102 is designed to be used in situations in which higher volume (or lower volume) exudates are expected. For example, wound dressings 101 designed for more chronic wounds or incisional wound dressings can be made thinner with smaller internal pathways through the wound contact layer 102 to reduce the thickness of the dressing. The smaller internal pathways may be appropriate for lower exudate wounds because there is less concern for clogging of the pathways.

In some implementations, the wound dressing 101 is directional or non-directional depending on intended use of the dressing. In some implementations, the wound dressing 101 is configured with a particular side of the dressing intended to be in contact with the wound surface (for example, where debridement is intended or where one side is coated in a therapeutic compound, a particular surface may be intended to be placed in contact with the wound bed 114). In some implementations, the wound dressing 101 or wound contact layer 102 is designed to be placed either side down (i.e., with either side facing the wound bed 114) with no change in function of the wound dressing 101. When either side can be positioned “face-down” in the wound bed 114 with no change in functionality, any potential human error regarding placement of the wound dressing 102 in the wound is eliminated. In conventional wound dressings 101 where one side of the device is intended to placed “face-down” in the wound bed 114, accidental misplacements of the wound with the wrong side down can cause delay in treatment or worse. If only one side of the wound dressing 101 is functional (and even worse if the opposite side would be detrimental to the patient if positioned to be wound-facing), then a human error in dressing placement could result in an inferior healing outcome. The design of wound contact layer 102 can advantageously be symmetric so that either side can be placed in a wound-facing position. In some implementations, the wound dressing 101 includes one or more layers in addition to the wound contact layer 102, for example, one or more perforated layers, one or more irrigation layers, one or more suction layers, or a separating layer. In some implementations, the one or more additional layers are formed above the wound contact layer 102 between a top of the wound contact layer 102 and the bottom of the sealing layer 104. In some implementations, the one or more additional layers are formed below the wound contact layer 102, between the wound contact layer 102 and the wound bed 114.

In conventional systems, standard negative pressure techniques do not allow for placement of negative pressure wound dressings on sensitive tissue such as bone, tendon, or nerve without a barrier in place, due to the potential for tissue ingrowth and drying effects of these sensitive tissues. However, wound contact layer 102, formed from a polymeric material, does not extract fluid from adjacent tissue in the way that conventional wound dressings including sponges do. Additionally, as described above, a polymeric material wound contact layer 102 can reduce or inhibit tissue ingrowth or does not stick to sensitive tissue. A more slippery contact surface of the wound contact layer 102 can be designed for application in wounds where these sensitive tissues are exposed, or a specific coating can be placed on the wound contact layer 102 that provides a slippery surface or site-specific tissue protective coating.

In some implementations, a traditional collection canister is not utilized for collecting exudate removed from the dressing. Instead, absorptive material is used within the system to collect removed exudate. For example, in some implementations, absorptive material is positioned within the dressing 101 itself in a position where it is separated from the wound surface and does not interfere with or restrict the flow of fluid through the dressing 101. In some implementations, absorptive beads or coated tubing is used to collect fluid within the dressing 101 while large flow passageways are still provided. Absorptive materials may be separated from the wound surface by a unidirectional flow barrier to prevent or reduce backflow of fluids toward the wound surface, and may be formed as a collection area in the tubing itself (rather than at the pump). In some implementations, gravitational forces can be used to separate fluids from gas across absorptive material to dry the air and remove fluid from the mixture of fluid and gas in the exudate as it is passed over highly absorptive powders (including spheres, rods, cones, or cubes).

### IV. Description of the Suction Dome

As described above, FIG. 1 illustrates a wound dressing 101 including a suction dome assembly 109 having inlet tubing 110 to provide inflow of irrigant (or therapeutics) to the wound dressing 101 and outlet tubing 112 for providing suction for negative pressure at the wound bed 114 and removal of exudate from the wound bed 114. Components and functions of the suction dome 109 are further described in FIGS. 3A-H which illustrate views of an exemplary docking port 300 and suction dome 301.

FIG. 3A shows a view of a docking port 300 formed in the top of a wound dressing 101 including outer ring 302, central suction port 308, external tubing 310A-D, internal tubing ports 312A-D and an irrigation well 306. The docking port 300 is formed as part of the wound contact layer 102. In use, the wound contact layer 102 including docking port 300 is positioned in the wound bed 114 and sealing layer 104 is positioned over the top of the docking port 300 and extending onto the surface of the patient's skin 116 surrounding the wound. The portion of the sealing layer 104 overlaying the docking port 300 is cut away, so that the suction dome 301 (described below with regard to FIG. 3H) can be engaged with the docking port 300.

External tubing 310A-D penetrates the outer ring 302 and ends at internal tubing ports 312A-D. In some implementations, internal tubing ports 312A-D may extend further into the center of the space within the outer ring 302 (i.e., further toward the port 308). In other implementations, internal tubing ports 312A-D are formed as ports in the internal surface of the outer ring 302. External tubing ports 310A-D are in fluid communication with the large-diameter passageways of the wound contact layer 102 to provide irrigant to the wound bed 114. In some implementations, the external tubing ports 310A-D extend throughout the wound contact layer 102 (in addition to or in place of the large-diameter passageways described above). In such implementations, the external tubing ports 310A-D can be radially arranged throughout the wound contact layer 102 or can be arranged in a leaves and veins pattern, for example as depicted in FIG. 2. The central suction port 308 is raised relative to a bottom surface of the docking port 300, so that an irrigation well is formed between the inner surface of the outer ring 302 and the outer wall of the central suction port 308. Irrigant can collect in the irrigation well before entering the internal tubing ports 312A-D and being distributed within the wound dressing 101 by external tubing ports 310A-D.

Outer ring 302 is illustrated as a circular wall with a top edge 304, though in other implementations the outer ring 302 is formed as a wall having another shape such as oval or any other suitable shape. In some implementations, the outer ring 302 has a height of 2-3 mm, 0-2 mm, 2-4 mm, 4-6 mm, or 5-10 mm, or any other suitable height. In some implementations, the outer ring 302 has a tapering wall, such that a diameter or surface area of the outer ring 302 at the top wall 304 is larger or smaller than a diameter or surface area of the outer ring 302 where it is coupled to the bottom surface of the docking port 300. In some implementations, top edge 304 is a pointed edge or a rounded edge rather than a flat edge as portrayed. In some implementations, top edge 304 is formed with a groove designed to mat with a dome portion (described below with regard to FIG. 3H). Central suction port 308 is illustrated as being positioned in a center of outer ring 302 but can be positioned anywhere within outer ring 302. In some implementations, the top edge 304 of the outer ring 302 has a sharp portion so that when the sealing layer 104 is positioned over the docking port 300, the sealing layer 104 can be easily cut away to expose the docking port 300.

In some implementations, docking port 300 is formed separately from the wound dressing 101 and is designed to be positioned on a surface of the wound contact layer 102 or directly on a surface of the sealing layer 104. In such implementations, the external tubing ports 310A-D may be omitted in favor of other mechanisms for providing irrigation to the wound contact layer 102.

FIG. 3B shows another view of docking port 300. In the docking port 300 of FIG. 3B, only two external tubing 310C-D are illustrated. In such an implementation, external tubing 310C can supply suction and tubing 310D can supply irrigation, or as described above, external tubing 310C-D can include multiple channels such that irrigation and suction can be provided with both of external tubing 310C and external tubing 310D. In some implementations, docking port 300 has only one connected external tubing 310. In some implementations, docking port 300 has two, three, four, six, eight, ten or more external tubing 310.

As described above, at least one of external tubing 310A-D is in fluidic communication with collection canister 106 (or flexible bag forming collection canister 106, as also described above) for collecting exudate or other liquids removed from the wound bed 114 with suction. As such, the external tubing 310A-D is configured so that fluids, either liquid or gas, can flow to the collection canister 106.

FIGS. 3C-G illustrate some implementations of a suction dome 301 for use with docking port 300. In some implementations, docking port 300 with external tubing 310A-B can be used with a dressing 311 having a central core 313 and a plurality of arms 315 extending radially outward around central core 313. Each arm 315 can include an internal pathway configured for fluid irrigation to a wound or for fluid suction from the wound. Unlike the leaf-like structure of dressing 200, dressing 311 can have a radial design with the plurality of arms 315 extending outward in a circumferential pattern from central core 313 to an outermost edge of dressing 311. In such implementations and others, docking port 300 can be removably coupled to central core 313 to facilitate irrigation and suction through the plurality of arms 315 using external tubing 310A-B. Docking port 300 and dressing 311 can be used with a wound filler 317 that is cut to size to fit a particular wound. In some implementations, the wound filler 317 can include a non-adhesive or backing layer that can be removed to expose an adhesive layer. In some implementations, suction dome 301 can be coupled to the central core 313 during manufacturing. In other implementations, the suction dome 301 can be coupled to the central core 313 after the dressing 311 is cut to size for use with a particular wound.

FIG. 3H illustrates other implementations of suction dome 301 for use with docking port 300. In these implementations and others, suction dome 301 includes irrigation tubing 316 with outlet 318, suction tubing 324 engaging inner bell chamber 322 at port 326, and adhesive skirt 320. Inner bell chamber 322 is designed to fit over the central suction port 308 when the suction dome 301 is engaged with the outer ring 302. Irrigation tubing 316 provides irrigant to the irrigation well 306 through outlet 318, which can be suspended above the irrigation well 306 so as not to be submerged in irrigant. Suction tubing 324 applies suction to the central suction port 308 through the top of the inner bell chamber 322 at port 326 and applies negative pressure at the wound bed 114. The suction dome 301 connects the wound contact layer 102 to the suction ports via the docking port 300.

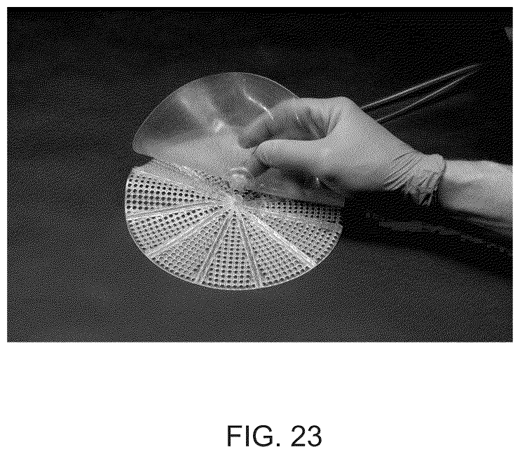

The suction dome 301 locks securely to the docking port 300, so that irrigation well 306 is fully enclosed to form an “irrigation chamber” and a “vacuum chamber” is formed by the connection of inner bell chamber 322 and suction port 308. In some implementations, the suction dome 301 includes a ridge 303 that fits into the grooved top of the top edge 304 of the outer ring 302 wall. Alternatively, the docking port 300 can include a trench, depressed area, detents, lip, or other mechanism for engaging with the suction dome 301. In some implementations, the suction dome 301 can be a docking type mechanism that fits by suction or friction into the docking port 300 built into or on top of the wound contact layer 102. For example, in some implementations, the suction dome 301 lacks a ridge 303 and instead is friction fit to the outside surface of outer ring 302. In some implementations, the suction dome 301 is coupled to the outer ring 302 by a mechanism similar to the application of the top layer of the ClearAp to the lower layer, as illustrated in FIG. 23.

In some implementations, the ClearAp (illustrated in FIGS. 3C-3G and 23, for example) can be configured to improve flow, reduce material usage, and facilitate drape adhesion and manufacturing. For example, rather than the top layer of the ClearAp covering the entire surface of the wound contact layer, the top layer, which may at least partially incorporate the suction dome 301, can have a reduced diameter compared to other embodiments. Wound visibility can be improved in such implementations because the top layer does not extend to the edge of the dressing (e.g., dressing 311), and therefore does not block the wound. Additionally, such implementations can make the wound softer or more compliant, and enable the dressing to be cut to size with ease. In some implementations, the flow or irrigation pathways (e.g., flow pathways defined by arms 315) can still be configured to transmit fluid because an adhesive layer can at least partially form a top surface of the flow pathways when placed under suction or negative pressure. Such implementations may also enable ease of applying the adhesive layer to the flow pathways.

In some implementations, the suction dome 301 is fit to the docking port 300 by a bulb-type mechanism to lock into one or both of the central suction port 308 or the irrigation well 306. In some implementations, the inner bell chamber 322 is fit to the central suction port 308 by suction or friction. In other implementations, the inner bell chamber 322 is fit to the central suction port 308 by a ridge, detents, a groove, or other suitable mechanism.

In some implementations, positioning the suction dome 301 over the docking port 300 includes cutting the sealing layer 104 so that the docking port 300 is exposed. The docking port 300 can include a trench or depressed area that would facilitate sharp cutting of the sealing layer 104 and would act as a guide to accurately cut a hole in the sealing layer 104 to accommodate the suction dome 301. Once the docking port 300 is exposed through a hole in the sealing layer 104, the suction dome 301 can be securely connected to the docking port 300. Once the suction dome 301 is connected to the docking port 300, the adhesive skirt 320 is positioned over the top of the sealing layer 104 to provide an air-tight seal to allow the application of negative pressure to the wound. In some implementations, the suction dome 301 and docking port 300 are positioned anywhere on the dressing. In some implementations, the suction dome 301 and docking port 300 are positioned in a center of the dressing.

The suction dome 301 delivers irrigation into the irrigation well 306 through the irrigation tubing 316 and provides suction to the central suction port 308 through the suction tubing 324. The suction could be provided so as to be deep or superficial and the irrigation could be opposite. For example, if the dressing 101 has multiple layers, the suction and irrigation can be delivered to different levels of the dressing 101. For example, the irrigation can be delivered to the layers that are deep within the dressing (e.g., on the wound surface) and suction can be delivered to the higher levels within the dressing. The direction of the irrigation can be directed via its location of delivery as well as its removal. For example, the direction of irrigation can be similar to the method used in the ClearAp dressing, where the irrigation is delivered to the periphery and it is sucked back up in the middle of the dressing requiring the fluid to travel across the wound before being sucked back up. In some implementations, the suction is provided cyclically. Although in FIGS. 3A-H, the suction chamber is formed in a center of the suction dome 301 and docking port 300, the suction dome 301 and docking port 300 are designed to have other arrangements of irrigation chamber and suction chamber. In some implementations, the suction dome 301 and docking port 300 are arranged so that the suction chamber is below the irrigation chamber, or so that the suction chamber is above the irrigation chamber. In some implementations, the irrigation chamber occupies one half of the suction dome 301 and docking port 300 (for example, one half of the circular structure) and the suction chamber occupies the other half. In such implementations, the suction dome 301 and docking port 300 can include a locking mechanism where the suction dome 301 and docking port 300 are only locked into a position when the appropriate orientation is achieved so that the irrigation tubing 316 is aligned with the irrigation chamber and the suction tubing 324 is aligned with the suction chamber. In some implementations, a lily pad type sealing layer around the tubing would create a seal over the docking port 300 rather than the suction dome 301 as shown. This would allow for customizability and reduced cost of the dressing while also allowing for outpatient pricing.

In some implementations, the docking port 300 is positioned so that it extends from the top side of the wound contact layer 102 and is above the sealing layer 104. In other implementations, the docking port 300 is inset into the wound contact layer 300 and is below where the sealing layer 104 will be applied. In some implementations, the docking port 300 can be a low profile port built into the dressing itself so there is no elevation above the surface of the wound contact layer 102 and no pressure point. In some implementations, a built-up cushioning part and/or mound is formed around the docking port 300 to protect the pressure point and to prevent compression of the docking port 300 toward the wound bed 114. In some implementations, the irrigation tubing 316 and vacuum tubing 324 are connected to the suction dome 301 at a right angle or are connected to the suction dome 301 with a curved or oblique entry to the suction dome to prevent or reduce tenting or kinking of the tubing.

In some implementations, the suction dome 301 can include one or more suction chambers. The suction chambers can be arranged in one or more layers of the wound dressing 101, or multiple suction chambers can be arranged in the same wound contact layer 102. In some implementations, multiple suction domes 301 can be applied to the same wound dressing 101, for example if the wound dressing is large. In some implementations, multiple suction domes 301 can be connected in series or parallel back to a single in flow or out flow source. In some implementations, irrigation of the wound surface is performed using two or more suction domes 301, with a first dome functioning as an in-flow and a second dome spaced apart from the first dome and functioning as an out-flow. The use of two or more suction domes improves the ability of a medical professional to observe irrigation and allows for high-flow volume through the dressing with low resistance.

In some implementations, the suction dome 301 includes additional tubing and can have a multitude of inlet ports, outlet ports, filters, pressure release valves or other functional features. For example, in some implementations, the suction dome 301 can have one, two, three, or more ports or portals. Each of the multiple ports of the suction dome 301 can be designated for one or more purposes. Each port can be designed for a single or multiple purposes depending on the desired function. For example, the ports can function to provide one or more of in-flow, out-flow, release valves, or dead-space prevention. In various implementations, the ports can provide one or more of gas, liquid, or solid to the irrigation well 306 or wound bed 114. In some implementations, the suction dome 301 can include a holding tank or well (not pictured) for allowing chemical reactions to occur at the wound bed 114, for example to introduce heat or cold to the wound bed 114. In some implementations, the irrigation well 306 can include heating coils or other circuitry to heat an irrigant for distribution to a wound. In other implementations, the irrigation well 306 can include refrigeration circuitry to cool an irrigation for distribution to the wound. The holding tank or well can include a port that is controlled by an on/off switch that opens a flap of the port (for example, controlled by the EVR 107). In this way, the application of chemical reactions or hot/cold fluids/gases can be regulated.

In some implementations, the suction dome 301 includes multiple ports to allow for filtered atmosphere, fluids, or gases to be provided to the wound bed 114. Portals designed to supply fluids can generally be larger than ports designed to supply gases, which can be restricted based on the diameter of the internal lumen of the port or tubing. Additionally, restrictors can be incorporated in different forms. In some implementations, a resealable flap can be used to start or stop flow through a port or lumen. In some implementations, a dial or adjustable flow restrictor can also be used. Different filter characteristics can additionally or alternatively be used to limit or increase flow. Additionally, filters can screen out microbes (i.e. HEPA filter or other types of filters), so that gas or ambient air flow does not create a new access for microbes to the sealed dressing space and so that wound contamination is prevented or reduced. In some implementations, nebulized (humidified) gases can be used to prevent wound desiccation or drying out.

In some implementations, the suction dome 301 includes an area of the dome that connects to the irrigation pathways allowing for repeatable injections of medicaments or therapeutic compounds through the suction dome 301 (similar to a port catheter). The material of the suction dome 301 can be soft, flexible, or self-healing in order to allow for repeated injections without creating a leak or loss of seal. The needle can be passed through the suction dome 301, and once the needle is removed the suction dome 301 is still air-tight preventing evaporation or leaking and allowing continued application of negative pressure. In some implementations, a rubber or alternative material could be used to form the suction dome 301 to allow repeat insertions of a needle into the suction dome 301. This design would allow for IV's to be attached to the suction dome 301, or injections of limited medications or chemicals into the inflow tubing without a specific port or tubing for inflow.

In some cases, the suction dome 301 or tubing connection to the dressing 101 forms a pressure point, especially where used with a non-compressible dressing (such as dressing 101). In some implementations, the suction dome 301 is surrounded by padding, for example around the dome, on top of the dome, or under the dome, to offload pressure points. In some implementations, the dressing incorporates additional padding under the suction dome 301 where it attaches to the sealing layer to reduce pressure on the wound caused by pressure of the suction dome 301. In some implementations, padding used to prevent or reduce pressure points incorporates one or more of an open air foam padding and a gel material. In some implementations, the dressing includes indentations for accommodating tubing to reduce prominence of the tubing above the dressing.

In some implementations, the suction dome 301 and the vacuum tubing 324 can be a source of pressure when a patient lies on at least one of the dome 301 and the tubing 324. In some implementations, the suction dome 301 can be converted to a flat collapsible design. In some implementations, the vacuum tubing 324 can be designed to fan out across an area over a wound filler or separating tab. In some implementations, the suction dome 301 can comprise a polymer material connected between a top end and a bottom end along opposing edges (e.g., a sealed casing or tube). In some implementations, an internal wall of the lumen can include one or more bumps (e.g., gel bumps), ridges, channels, or other uneven surfaces to prevent the lumen from collapsing under negative pressure, thereby maintaining consistent flow through the lumen. In such implementations or other implementations, there is no hard tubing and the suction dome 301 is not elevated or stiff.

In some implementations, a separate material can be used to couple the wound filler to the vacuum tubing 324. Similar to other implementations, this separate material can be connected between top and bottom ends along opposing edges to create a sealed, non-stiff structure. In such implementations, an internal wall of the lumen can include one or more bumps (e.g., gel bumps), ridges, channels, or other uneven surfaces to prevent the lumen from collapsing under negative pressure, thereby maintaining consistent flow through the lumen.

In some implementations, external padding can be used to cover the dressing 101 or vacuum tubing 324 to prevent potential pressure injuries to patients. In some implementations, the external padding can be positioned around the vacuum tubing 324, for example above and/or below the tubing 324. In some implementations, the external padding can be made out of foam or gel (non-compressive), or another similar material. In some implementations, the external padding can be positioned outside the area placed under negative pressure. In some implementations, adhesive can be applied to at least part of the external padding, such as a padding side, so that the external padding can be adhered to the dressing 101 and/or to the patient. In some implementations, a padding with a torus shape can be positioned around the suction dome 301, the vacuum tubing 324, and/or the irrigation tubing 316.

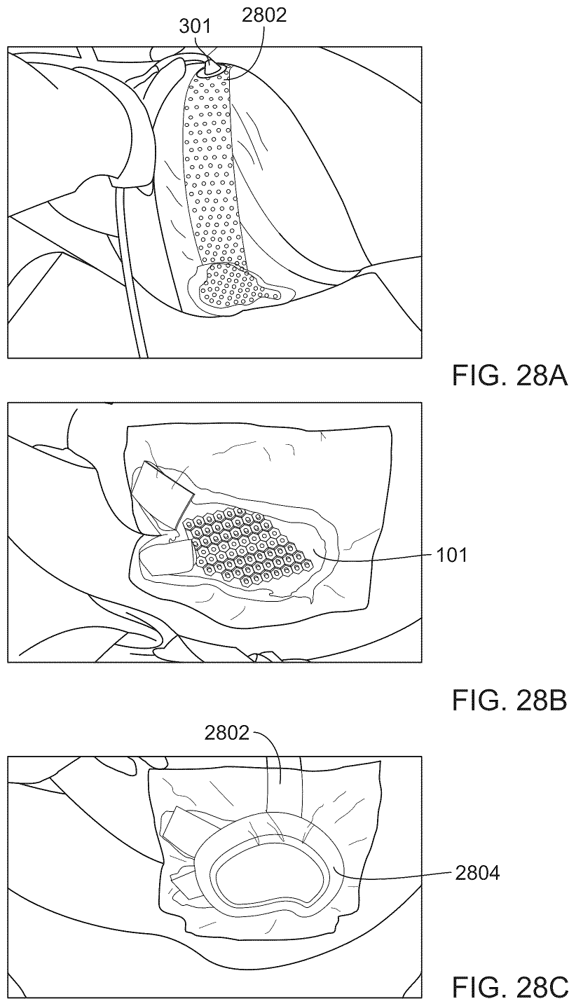

Referring now to FIGS. 28A-C, in some implementations, a bridging material 2802 can be placed on a top surface of a sealing material to create a seal against the wound. In some implementations, the bridging material 2802 can comprise a thin piece of foam. In some implementations, at least part of the sealing material or another non-compressible thermoplastic or polymer pliable material can be used to provide minimal resistance to flow and minimal positive pressure with the bridging material 2802. In some implementations, the bridging material 2802 can be non-compressible to enable unrestricted flow through a lumen of the bridging material between the suction dome 301 and a wound. This can enable bridging of the wound filler or separating tab to an area away from a position where the patient's body weight presses on the wound.

In some implementations, negative pressure can be transmitted to evacuate exudate from a wound while still using a low or reduced negative pressure setting (e.g., in the range of −20 mmHg to −100 mmHg). Still referring to FIGS. 28A-C, in some implementations, the bridging material 2802 can be placed on top of the wound sealing layer to prevent skin maceration and other adverse conditions. The bridging material 2802 can be covered by an additional sealing layer (e.g., additional padding 2804) to create an additional seal against the bridging material. In such implementations and others, the suction dome 301 can then be placed on top of the bridging material 2802 at a position away from a weight bearing area created by the patient's body. In some implementations, the bridging material 2802 can include an adhesive side configured for application to the patient. In some implementations, the lumen or flow pathway of the bridging material 2802 can be self-contained and sealed or airtight on top of a foam or gel layer of the bridging material. The bridging material 2802 can be cut to a desired length and placed over an NPWT dressing (e.g., dressing 101) and extended longitudinally to a location where a suction dome is provided. The foam or gel layer can be external to the negative pressure area and thus can act merely as a pad or pressure prevention material during use in wound therapy. In some implementations, additional padding 2804 can be placed over a top surface of the NPWT dressing, in addition to the bridging material 2802 placed over the dressing. This additional padding 2804 (e.g., foam, gel, other padding) can act as a pressure reducer without being placed under the negative pressure forces applied to the wound.

### V. Description of the Leaf-Style Dressing

In some implementations, a dressing with characteristics as described with regard to FIG. 1 is designed as a site specific dressing which is shaped and designed for use on a specific portion of the body or with a specific kind of wound (see e.g., FIGS. 4A-B, 5-6, 7A-E, 8-9, 10A-C, 11A-C, 12A-C). In some implementations, site specific dressings are designed to be similar to oven mitts or stockinette's used in the operating room and are designed to cover intact skin and/or open wounds. Site specific dressings can be designed to promote healing through the application of negative and positive pressure through the dressing to the wound surface, and included flow pathways for introducing gases, chemicals, and changes in temperature (e.g., to treat or induce frostbite). In some implementations, site specific dressings are designed to provide an air-tight seal over the wound, and only the seal at the end of the dressing (the proximal aspect of the dressing) would be needed.

For example, although the wound dressing 101 is illustrated in FIG. 1 as a single dressing, the features described with regard to the wound dressing 101 of FIG. 1 can be applied in dressings that include multiple connected wound dressings. For example, the features of wound dressing 101 can be applied in a leaf-style dressing that can be used in treatment and dressing of wounds that have multiple cavities that may not be accessible by a single wound filler placed at the top of the wound. Such multiple cavity wounds need discrete elements of the dressing to be directly placed into each of the cavities of the wound. The leaf-style dressing is a series of dressings (e.g., multiple wound contact layers 102) having irrigation and/or suction systems that are connected to one another fluidically with a hub-and-spoke construction. Use of leaf-style dressings using wound contact layers 102, would overcome a critical, potentially lethal drawback of conventional foam/sponge-based wound dressings, which treats these deep cavity wounds by placing several independent and non-affixed pieces of foam/sponge in each of the cavities. The pieces of foam/sponge can be left behind at wound dressing changes and retained pieces of sponge can provide a nidus for serious infections. The hub-and-spoke construction of the leaf-style dressing would prevent the unintentional retention of dressing material in the wound.

A leaf-style dressing can allow for addition or subtraction of independent dressings or wound contact layers. For example, a leaf-style dressing can provide multiple wound dressings that are connected together but can be cut apart to fit the wound without impacting functionality of the irrigation or vacuum systems of the dressings. In some implementations, a leaf-style dressing can include coupling connections in the irrigation/suction tubing extending from the dressing to enable the addition of more fluidically connected dressings if necessary for treatment of a wound. These leaf-style dressings can be used across multiple different wounds or cam be applied in different layers within a single wound or within a complex wound. These dressings can have specific flow pathways for suction as well as irrigation, and therapeutics can be delivered through the irrigation pathways while suction pathways can be used to provide negative pressure.

For example, a leaf-style dressing can include a first dressing and a second dressing, each including a polymeric and transparent wound contact layer and an irrigation pathway through the polymeric wound contact layer. The first dressing and the second dressing are coupled to each other as described above. In some implementations, the irrigation pathway through the first dressing is fluidically coupled to the irrigation pathway of the second dressing by an irrigation tube. In some implementations, the first dressing is coupled to the second dressing by one tube including two or more lumens. In other implementations, the first dressing is coupled to the second dressing by two or more tubes. The multiple tubes or lumens allow for one dressing (e.g., the first dressing) to be coupled to an EVR and for the EVR to provide irrigation and suction so that irrigation can be passed from the first dressing to the second dressing, and extraneous irrigant, exudate, or other liquids can be passed back from the second dressing to the first dressing and out of the system. In some implementations, the first and second dressings are coupled by a tube or string, but each of the first and second dressings includes its own tubing for coupling to the EVR. As described above, the EVR (for example EVR 107) includes an inlet and an outlet for providing suction and irrigation, respectively. The EVR provides irrigation to the leaf-style dressing through a first irrigation tube and provides suction to the dressing through a second irrigation tube.

In some implementations, the first irrigation tube is connected from the outlet of the EVR to the irrigation pathway of the first dressing, and the second irrigation tube is connected from the irrigation pathway of the second dressing to the inlet of the EVR. In such implementations, a flow path is defined from the outlet, through the first irrigation tube, through the irrigation pathway of the first dressing, through the irrigation tube connecting the first and second dressings, through the irrigation pathway of the second dressing, through the second irrigation tube to the inlet of the EVR. In this implementation, the flow path moves from the EVR to the first dressing to the second dressing and back to the EVR. In other implementations, the first irrigation tube is connected from the outlet of the EVR to the irrigation pathway of the first dressing, the second irrigation tube is connected from the irrigation pathway of the first dressing, such that irrigation and suction flow in two flow paths, one extending from the EVR through the first dressing and back to the EVR and a second flow path extending from the EVR through the first dressing to the second dressing, through the second dressing back to the first dressing, and finally back to the EVR.

When formed from a polymeric material as described with regard to FIG. 1, these leaf-style dressings can be designed to be intentionally left in the wound where they can serve as a surgical implant that encourages ingrowth of local tissues to fill a deep cavity as the wound or tissue continues to heal around the dressing. For example, leaf-style dressings formed from silicone can be left in the wound as tissue scaffolding, while a sponge (for example as used in conventional wound dressings) cannot be left in a body as it will be a nidus for infection and tissue reaction. In some implementations, a disposable or permanent implant can be formed from a polymeric material and used as a dressing. In some implementations, suction and inflow tubing can attach to each of the leaf-style dressings for use in healing tunnel wounds. In some implementations, this design could act as a scaffolding which reduces risk of abscess formation by preventing a tunnel wound from closing in a way that would create a dead space. In some implementations, a leaf-style dressing is formed as a dissolvable or biodegradable structure made of collagen or other materials that would be incorporated into the patient as the wound and tissue grow into the dressing. In some implementations, the leaf-style dressing can be formed as a biodegradable sponge-type structure that encourages tissue ingrowth but is designed to transfer suction or incorporate irrigation prior to full ingrowth of surrounding tissue. In some implementations, tubing associated with the leaf-style dressing is connected through a bio-inert connector such as silicone, stainless steel, titanium, or TPE.

In some implementations, the leaf-style dressing includes a base dressing (for example dressing 101 of FIG. 1) to which “leaflet dressings” can be added. In some implementations, the leaflet dressings can be added by connecting tubing of the leaflet dressing to a port in the suction dome 301 (or suction dome assembly 109 in FIG. 1). In some implementations, the leaf-style dressing is manufactured and made available as a standard set of dressings including multiple connected leaves that can be cut off if not needed during placement of the dressing. For example, the standard set of connected leaves can include three leaves, five leaves, ten leaves, or any other suitable number of leaves. By providing a set of already connected leaves, the dressing can be adjusted during placement of the dressing to customize to the shape of the wound. In some implementations, the set of already connected leaflets are used on their own and include their own irrigation and suction system and port for connection to an EVR (for example EVR 107). In other implementations, the set of already connected leaflets are connected to the base dressing (for example dressing 101 of FIG. 1) by one or more tubing systems to provide irrigation and suction. In some implementations, the set of already connected leaflets including tubing systems allowing for bidirectional flow through single flow tubes or dual lumen tubes allowing for inflow in one path and outflow in the other path.

In some implementations, the leaf-style dressing includes an indicator extending from at least one of the dressings. The indicator acts as a “leash” which can be positioned at the wound surface after positioning the dressings in the wound. The indicator can aid in retrieval of dressings that are deep within the wound and may be difficult to find on initial inspection. The indicator can be built into the dressing as manufactured or can be added to a dressing component that may not be visible at the initial wound inspection. For example, an indicator can be added to the dressing 101 of FIG. 1 or to any of the dressings described below with regard to FIGS. 4-19 and 21-22. In some implementations, the indicator can be formed to have bright non-biological colors to stand out and be easily visible to a medical provider. In some implementations, the indicator can have large tags or appendages that draw attention to it. In some implementations, the indicator can be solid or a mesh or screen-type design to prevent dead spaces within the wound.

In some implementations, the docking station can also be designed to be off center or on one side at the edge. In this design, the contact layer can cover the wound and the docking station can be used with a bridging component to provide suction and inflow without the dome being placed over the dressing. One example where this is advantageous is with sacral pressure injuries where wounds are susceptible to pressure injuries from the suction dome or tubing.

### VI. Dressing as a Skin Substitute and/or in Grafts

In some implementations, the polymer-based wound contact layer 102 and wound dressing 101 described in FIG. 1 can be used to deliver skin grafts or skin substitutes. Conventionally, a negative pressure dressing or bolster dressing is placed over a skin graft (e.g., a split thickness skin graft, full thickness skin graft, allograft or skin substitute) to apply pressure to the graft onto the graft site. This conventional process for performing skin grafts includes placement of the graft and placement of the dressing over the graft in two separate steps: first the skin graft is applied, then the dressing is applied. In some cases, shear and lift-off (i.e. due to hematoma) prevents blood vessels from the host from growing into the skin graft to make it “take” and survive. The negative pressure or bolster dressing push the graft onto the graft site and prevent or reduce shear forces or lift-off from occurring. However, conventional dressings including a sponge allow ingrowth, so that when the sponge is removed the graft adhered to the sponge will be peeled away from the graft site, leading to grafting failure and need for more surgery or other advanced wound care. In some cases, a barrier is used under the conventional sponge dressing to prevent graft removal with sponge removal, but this adds additional steps to the procedure. Additionally, hematomas can separate the graft (lift-off) from the graft site and prevent new vessel ingrowth. The use of negative pressure wound therapy helps to remove blood pockets or hematomas from the graft site.

In some implementations, the semi rigid, non-compressible, transparent, polymer wound contact layer with large flow pathways can be advantageous for use in skin graft placement. With flaps or split thickness skin grafts (STSG), pressure can be detrimental to graft uptake and healing. Additionally, skin grafts can take in some areas and fail in others. The ability to provide low negative/positive pressure on the graft (e.g., +10 mmHg to +30 mmHg) can have a number of advantages. The low pressure can protect the healing graft from ischemic pressures and can prevent shear forces. The low suction and wide flow pathways can prevent hematomas from developing under the graft which can lift the graft off the wound bed and prevent graft uptake. In some implementations, transparency can enable graft monitoring through the dressing. The lack of fluid retention can prevent bacterial overgrowth which can cause graft failure.

A wound contact layer 102 formed from a polymer (for example, described in FIG. 1) can prevent tissue ingrowth, and can be used in the graft site or above the skin graft without requiring a barrier. The wound contact layer 102 can be placed directly over the graft site to promote healing. In some implementations, the wound contact layer 102 can provide negative pressure to compress the graft to the recipient site and can prevent or reduce the occurrence of hematomas. In some implementations, shear forces can be reduced by adding a coating to the wound surface, the graft surface, or to the wound-facing surface of the wound contact layer 102. In some implementations, the attachment chemical between the dressing and the graft can become a lubricant once it breaks down or is exposed to fluid. A coating to provide lubrication such as hyaluronic acid or petroleum jelly or other treatments can be used to reduce adhesion and minimize shear forces. A wound contact layer 102 that has a smooth surface and is formed from a polymer with large-diameter passageways (e.g., without pores) reduces opportunity for ingrowth into the dressing and can improve skin grafting success when used to deliver the bolster effect and to provide negative pressure for removing hematoma.

In some implementations, the wound contact layer 102 and the tissue graft can be delivered to the wound together. In such a system, an allograft (i.e. from a cadaver or xenograft from another species) skin is adhered to the wound contact layer 102. The allograft skin is adhered to the wound contact layer 102 so as to remain adhered through packaging, storage and placement of the dressing, but to allow reliable release when placed in the moist wound environment. In some implementations, the allograft is adhered to the dressing by a “glueing” agent, like corn starch, which will dissolve in the moist wound environment. By placing the allograft and dressing in the wound together, the dressing can be perfectly matched to the skin graft area, and the procedure can be accomplished with fewer steps. Allograft or xenograft may be more successful using a system with the graft and dressing delivered to the wound together, and such a product could obviate the need of autograft harvest from the patient themselves. Traditionally, allograft and xenograft has been less effective, than autograft, but are necessary where harvest morbidity occurs and for burn patients with large areas of burn where there may not be sufficient autograft. These composite graft and dressing composites have the advantage of being ready for use directly from the package, could be stored in a controlled temperature environment until they are brought to the operating room for use. A wound contact layer 102 with clear/transparent construction would have the additional benefit that it could be placed over the graft site to determine the size of the wound (visible through the dressing), after which the graft and dressing could be cut to size and shape together to fit the wound. Then the dressing and graft would be applied in one step, with no need for stapling or suturing the margins of the graft to secure it to the site, because the dressing and its adhesive drape would fulfill this function.

In some implementations, the wound contact layer 102 can be used to deliver a pre-manufactured synthetic skin graft, xenograft or allograft, as described above, and in other implementations the wound contact layer 102 can be used to deliver grafts including biological skin (human, amniotic, pig, fish or other animals) or synthetic (Collagen, hyaluronic acid . . . ) skin substitutes. In some implementations, growth factors as well as other therapeutics are coated on the wound contact layer 102 or in the adhesive material between the dressing and the graft to enhance receptiveness of the wound to the graft and provide treatment of the wound bed.

Combinations of wound contact layer 102 and grafts for delivery together can be especially helpful for burn patients where large sheets of skin substitutes can be used. In some implementations, the polymer of the wound contact layer can be used to hold sutures or staples instead of the very flimsy allograft tissue. Additionally, the wound contact layer 102 can be used to deliver irrigation to the skin or to defects in the skin minimizing water loss and preventing or reducing skin dry out. This is especially important for burn patients receiving grafts, as dehydration can be a serious concern due to lack of intact skin over significant amounts of the body.

In some implementations, the wound contact layer 102 described with regard to FIG. 1 is used to deliver a skin graft slurry containing cells, biologics or collagen mixed into a congealed slurry for delivery through the irrigation system of the dressing when a wound has progressed to an appropriate stage of healing. For example, the slurry could be delivered to the wound bed 114 where it could congeal, harden, or solidify over the wound surface due to biological factors naturally occurring within the wound or through reaction with a reagent delivered to the wound bed 114 through the wound contact layer 102.

In some implementations, any graft, dermal regenerative templates or skin substitute or enhancer can be used with the wound contact layer, including autograft, allograft, xenograft, integra (bovine & shark products & silicone sheet), Matriderm (bovine), PELNAC (Porcine & silicone sheet), Acell (Porcine bladder mucosa), NovoSorb BTM (Polyurethane foam), and Primatrix (Bovine) or other types of skin substitutes such as fish scales. In some implementations, the wound contact layer 102 and graft combination can additionally be used with a skin expander, for example a synthetic skin expander. In some implementations, the wound contact layer 102 is used as a skin expander. In some implementations, the wound contact layer 102 or the synthetic skin expander is a dissolvable skin expander. For example, a dressing or expander formed from a collogen product could be used to provide a scaffolding for skin cells to migrate into the area and could dissolve over time. In some implementations, vitamins such as vitamins E or D and other factors known for assisting in prevention of scarring can be supplied to the wound and graft, including being supplied through the irrigation system of the wound contact layer 102 or through a coating on the wound contact layer 102. In some implementations, steroids can be provided to inflammatory wounds in a similar manner.

In some implementations, the dressing 101 may be used as an interpositional implant between bones or joints. For example, the thumb carpometacarpal joint is often associated with arthritis. In many instances, surgery is required where the trapezium is resected and either a tendon or suture system is used to suspend the thumb metacarpal on the index metacarpal. If a tendon is used, the excess tendon is stuffed into the trapezium resection site to prevent abutment of the two bones. In this example, the dressing 101 may be used as a interpositional graft to be a second barrier between the bones for abutment prevention.

### VII. Dressings for Use in Treating Incisional Wounds

As described above with regard to FIG. 1, a dressing 101 can provide therapeutics to a wound bed 114 through coatings of the wound contact layer 102 or through the irrigation system (e.g., FIG. 2). The ability to provide therapeutics to a wound bed can be useful in aiding healing of the wound bed 114, or in targeting deleterious cells within the wound bed 114. FIGS. 4A-C illustrate steps of a method for using an exemplary wound dressing to treat a tumor bed. FIG. 4A shows tumor 404 existing under skin 402 and with surrounding margin of cancer cells 403. FIG. 4B shows the removal of tumor 404 from the tumor bed 406 through an incision in skin 402. The tumor bed 406 includes surrounding margin of cancer cells 403 that extend beyond the cavity (e.g., tumor bed 406) from which the tumor 404 was removed. FIG. 4C shows the treatment of the wound using a dressing to heal the wound and deliver chemotherapy to kill microscopic cancer cells in the surrounding margin of cancer cells 403.

Some conventional incisional dressings can utilize an absorbent and collapsable material to absorb any fluid from the wound. This absorption can enable bacterial overgrowth. It can also collapse which then requires increased negative pressure. A polymer, non-collapsable dressing that does not absorb fluid can eliminate or reduce these issues. Low pressure can be utilized, and a collection canister or highly absorbent material can be designed to trap fluid within the tubing or at the pump away from the wound. These designs can reduce ischemic pressures in some implementations. Such designs can also remove fluid from the wound surface thereby reducing bacterial colonization. Improved healing can occur with low pressures, dry environments, low bacterial colonization, and improved flow with low positive pressure application according to some implementations.

In FIG. 4C, a dressing 408 is positioned in the wound and sutures 416 are used to close the skin 402 around the wound around the dressing 408. Inflow/outflow tubing 414 extends from the dressing 408, through a skin penetration point 412 to provide irrigation and/or suction to the dressing 408 through tubing system 410. The dressing 408 delivers chemotherapeutic agents to specifically kill cancer cells in the margin of cancer cells 403 that surround the wound. The dressing 408 is used as an indwelling closed system for controlled local delivery of medicinal agents to the tissue in contact or proximity to the dressing 408. In some implementations, depending on the shape of the tumor bed 406 and location of remaining cancer cells, the dressing 408 can be surface-located instead of being positioned within the wound. The dressing 408 for delivery of chemotherapeutic agents can be used in open or closed wounds. For use in a closed wound, the dressing 408 has a shape to cover the base of the resection bed (e.g., tumor bed), to deliver therapeutics under a closed incision. When treatment has ended, the dressing 408 can be pulled from the wound through a perforation in the skin.

By using the dressing 408 for chemotherapy deliver, continual or intermittent direct delivery of medications to the wound bed is enabled to manage either non-resectable cancer tumors or tumor beds where a clear margin was not obtained (i.e. there is retained tumor in the wound bed). In some implementations, general cytotoxic agents like phenol are delivered to the tumor bed initially, followed by a neutralizing agent. In some implementations, cancer-specific agents can be delivered to the tumor bed to kill cancer cells in the wound without harming non-cancerous cells in the wound. This design could be used to remove other cell types aside from cancer cells as well. For example, medication delivery through the dressing 408 can be used to promote desired cells while inhibiting or killing undesired cells. These cells could be autologous or allogenic or non-human or infected human cells. In some implementations, the dressing 408 can be used to administer multiple chemotherapeutic treatments to the tumor bed. As an example, radioactive iodine can be used to kill cancerous thyroid cancer cells following thyroidectomy for cancer.

In some implementations, the dressing 408 can be shaped and sized so as to be specialized to direct the chemotherapeutic agents to the cancer cells surrounding the tumor bed or wound bed to protect the healthy cells while killing cancer cells. For example, in the treatment of brain cancers or other types of cancer where surgical excision is difficult or impossible, a specialized dressing can be used to direct chemotherapeutic agents to the cancer cells. In some implementations, a dressing 408 as small as a drain or as big as a 20 cm or larger dressing can be utilized to repeatedly deliver chemotherapeutic medications and neutralizing medications over an extended period of time (for example, days to weeks).

In some implementations, in addition to chemotherapy, the dressing 408 also delivers irrigation fluids to cleanse wounds and other medicinal agents aimed at improving wound healing, as described above with regard to FIG. 1. In some implementations, dressing 408 is a bladder that can be inflated to fill the tumor bed, or deflated as the wound heals.

In some implementations, the dressing 408 can be used to supply contrast agents to the wound through the irrigation tubing 410 to visualize complete coverage of the wound. For example, contrast agents such as gadolinium or iodine can be used for MRI or CT imagining or plain x-rays. The dressing may be designed to be MRI compatible by not including any metals, and can further be CT compatible by not including any metal or Ca++. In some implementations the dressing delivers contrast agents to the wound through the tubing to allow visualization of areas being covered by the irrigation or medication delivery system. In some implementations, the contrast is introduced to the wound bed before an MRI or CT scan are initiated to confirm that the entire wound bed area is covered by the irrigation and/or medication, for example, when cancer resection wounds are being covered by the dressing. In another example, contrast that is radio-labeled and able to target cancer cells can be supplied to the wound using the dressing 408, where they are allowed to bind to cancer cells so that radio-counters can detect bound labeling molecules to identify presence and amount of remaining cancer cells in the resection bed. PET scans can be used to evaluate where metabolic activity is occurring in the wound bed, and once particular areas of metabolic activity are identified, the dressing 408 can be used to deliver medications to the specific areas. In some implementations, the dressing 408 includes multiple arms of tubing 410 that extend into different areas of the wound. In some implementations, specific tubing 410 of the dressing 408 is used to deliver medications to specific areas of the wound bed. For example, a first tube can provide medications to a first quadrant of the wound, a second tube can provide medications to a second quadrant of the wound, a third tube can provide medications to a third quadrant of the wound, and a fourth tube can provide medications to a fourth quadrant of the wound. More or less tubing can be used to more accurately deliver medications and therapeutics to specific areas of the tumor bed. In some implementations, the therapeutic material can be mixed with a colored dye so that a provider can determine by visual inspection of the wound that the entire wound is covered by the therapeutic material. This dye can be biodegradable so as to not discolor the skin or soft tissue and so as not to damage the tissue, for example, methylene blue dye. In some implementations, a dye can be mixed with the therapeutic material that fluoresces under UV or black light to show coverage.

In some implementations, the dressing 408 can be bidirectional in that it can deliver therapeutics to the deep surface and to the superficial surface of the wound bed. The radial tubing 410 can have perforations along the length of the tubing so as to supply irrigation or therapeutics to both the deep and superficial regions of the wound. In some implementations, medications to promote or inhibit neo-vascularization can be delivered using the dressing 408 to promote or inhibit cell growth. By delivering particular medications to the tumor bed, apoptosis can be induced in specific cells causing cell death.

In some implementations, the dressing 408 can assist in wound bed monitoring to determine if cancer DNA is still being created in the wound bed (i.e. that cancer cells are still being killed). Outflow tubing 414 of the dressing 408 can suction exudate from the wound bed which can then be monitored. For example, the extracts from the wound bed (e.g., exudate) can be assessed for presence of viable cancer cells and/or cancer cell products (like proteins) and this information can be used to direct therapy. The exudate can also be assessed for bacterial presence and if detected the presence can be quantified to direct antimicrobial therapy and general wound care for infected or chronic open wounds. In situations where cancer cell DNA is known based on excisional biopsy or through needle biopsy, wound bed exudate can be monitored to observe when no more cancer DNA is in the exudate. For example, ELISA DNA sampling can be performed at routine intervals to determine when the tumor bed is cancer free.

In some implementations, the dressing 408 can be used to deliver a liquid radioactive material bound to a targeting protein or molecule that can be used to direct radiation therapy directly to the wound surface. The radioactive material can be in a powder form or a liquid form. The radioactive material can be applied for specific times and then can be flushed or neutralized by a reagent to stop the action, for example, by further irrigation through the dressing 408 or by providing additional neutralizing agents to the tumor bed through the dressing 408. This therapy can be repeated over multiple intervals until the desired level of treatment is complete, and the therapy can be controlled through the duration of instillation or bathing of the tumor bed with the radioactive material, as well as the number of treatments.

In some implementations, a leaf-type dressing system as described above can be designed to provide medication to multiple areas of the wound bed, depending on tumor location and orientation. In some implementations, each of the dressings of the leaf-type dressing system can include its own suction/irrigation tubing to allow for optimal flow through the wound bed, and to allow targeting of specific regions. In some implementations, the dressing 408 is designed as a linear type dressing that includes an inflow tube and an outflow tube, where the suction tube can be occluded to force medication out of the outflow tube. On the opposite side of the occlusion can be a suction port(s) that allows for the medication to be sucked back up. This would be ideal for tunneling or linear type tumors or wound beds. An example of this is a tape-type dressing including an outflow tubing that goes to the distal end of the tape-type dressing. The tape-type structure further includes fluid pathways allowing medication to travel through the outflow tubing after delivery to the tubing through the distal end of the dressing. IN some implementations, the proximal aspects of the tape-type dressing include a single suction tubing end or includes multiple suction tubing ends. Such a design allows medication to be delivered to the distal aspect of the dressing through the tubing, and then sucked back through the flow pathways up to the proximal suction tubing to allow for medications to be delivered through a deep or tunneling wound or cancer resection bed.

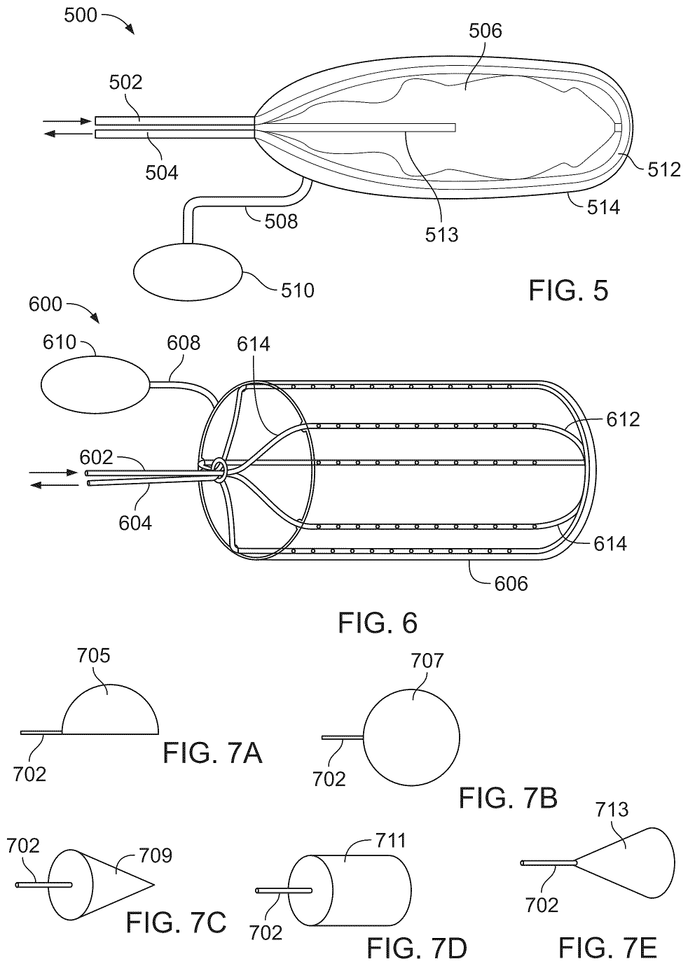

In some implementations, to remove the dressing 408 it can be pulled through the skin as the dressing 408 collapses on itself. Alternatively, a small incision can be made either in the Operating Room or at the bedside under local anesthesia to remove the dressing 408. Additionally or alternatively, the dressing 408 can be designed so that it can be collapsed to a smaller shape (for example like an umbrella) to become conical or cone-shaped to allow for easier removal through a small wound or hole. For example, FIGS. 5-12 show exemplary dressings that are expandable and collapsible to assist in tissue expansion and/or removal of the dressing from an incisional wound (e.g., a tunnel/tunneling wound). In some implementations, as shown in FIGS. 5-6 and 10-11, a dressing having an expandable cigar-shape can be used to treat tunneling wounds. In some implementations, the dressing is insertable into the tunneling wound in a compressed state and is expandable by a central bladder that can be inflated or deflated over time to allow for healing of the wound. In some implementations, the dressing further includes a wound filler that discourages tissue ingrowth.

While the dressings of FIGS. 5-12 illustrate the use of bladders, pumps, and mechanical expanders, other expansion mechanisms can also be used to expand and collapse a dressing for treatment of a wound, such as mechanical arms that rotate outward, electronic components that move relative to one another in response to instructions, haptic systems, motors used with impellers or fans, a system including a piston, a linkage and/or a solenoid, chambers for chemical reactions that produce gas or otherwise increase a volume of the chamber, or other suitable mechanisms.

FIG. 5 illustrates a system 500 including an exemplary expandable bladder 506 that can be deflated for removal. System 500 includes a bladder 506, peripheral tubing 512 coupled to inflow tubing 502, central tubing 513 coupled to outflow tubing 504, surrounding dressing material 514, inflation pump 510 and inflation tubing 508. The expandable bladder 506 is inflatable and deflatable using inflation pump 510 to supply (inflating) or remove (deflating) a liquid or gas to the bladder 506 through inflation tubing 508. Peripheral tubing 512 extends within the dressing material 514. In some implementations, dressing material 514 extends between the peripheral tubing 512. In other implementations, dressing material 514 is omitted. Peripheral tubing 512 provides irrigation and suction to the edges of the tunnel wound. Central tubing 513 is coupled to outflow tubing 504 and provides suction to the wound. Irrigation is supplied to peripheral tubing 512 through inflow tubing 502 and suction is provided to central tubing 513 through outflow tubing 504. In some implementations, the bladder 506 is incorporated within or adjacent to dressing material 514. In some implementations, the bladder 506 is formed from a polymeric material that discourages tissue ingrowth (for example the material of wound contact layer 102 in FIG. 1).

As shown, the therapeutic delivery system can be 3-dimensional and can have an elongated balloon or football shape. In some implementations, the bladder 506 has a shape designed to expand breast tissue before reconstruction surgery. The bladder 506 can be used alone or with coatings as described above with regard to dressings. In some implementations, the bladder 506 can be inserted into an incisional or tunnel would and used as a tissue expander. In some implementations, sequential air or liquid inflow and out flow to the bladder 506 can alternatingly expand and contract the bladder to assist in delivery of medicament to the entire wound bed. For example, the system can be used in breast cancer patients preparing for breast reconstruction surgery, where the peripheral tubing 512 can provide therapeutics (as described above) to the tumor bed while the bladder is expanded to stretch the skin and tissue for reconstruction.

FIG. 6 illustrates a system 600 including an exemplary inflatable bladder 606 for use in a wound bed. The system 600 includes a bladder 606, peripheral tubing 612 coupled to inflow tubing 602, central tubing 613 coupled to outflow tubing 604, inflation pump 610 and inflation tubing 608, and peripheral dressing 614. The expandable bladder 606 is inflatable and deflatable using inflation pump 610 to supply (inflating) or remove (deflating) a liquid or gas to the bladder 606 through inflation tubing 608. The bladder 606 is positioned within a peripheral dressing 614 that surrounds the bladder 606. The peripheral dressing 614 can be formed of materials described with regard to wound contact layer 102 in FIG. 1 and can similarly provide therapeutics through coatings or irrigation. Peripheral tubing 612 is part of the peripheral dressing 614 and provides irrigation to the edges of the tunnel wound. Central tubing 613 provides suction through the center of the bladder 606 and/or peripheral dressing 614. Central tubing 613 is in communication with outflow tubing 604. The bladder 606 includes a cavity to allow an insertion rod (not shown) to be inserted into a center of the bladder 606 to assist in placement of the bladder 606 within an incisional or tunnel wound in a deflated state. In some implementations, the central tubing 613 extends into the same cavity. In some implementations, the bladder 606 is disposed adjacent to a dressing (such as wound contact layer 102 in FIG. 1) rather than being completely surrounded by the peripheral dressing 614. In some implementations, the bladder 606 is used without a peripheral dressing 614.

In some implementations, the bladder 606 can be used to perform dialysis by introducing dialysis fluid using the inflow tubing 602 through the peripheral tubing 612. During dialysis of a wound, a medication is either removed or provided to the wound bed via osmosis or gradients created by fluid within the bladder. The bladder can be a porous or semi porous/permeable barrier/membrane for diffusion. The duration of the dialysis treatment can be modified based on patient requirements. The use of a bladder 606 for providing dialysis to a patient who already has an incisional wound eliminate the need for needles and injections or shunts to provide dialysis. In some implementations, the bladder can be used to perform dialysis as a treatment for poor kidney function, similar to peritoneal dialysis. Additionally, with the use of bladder 606 the dialysis fluid can be easily infused into the patient and removed from the patient without need for needles using the inflow tubing 602 and outflow tubing 604 (with peripheral tubing 612 and central suction tubing 613). Increased amounts of fluid removal can be performed using central suction tubing 613 and outflow tubing 604 and using the peripheral tubing 612 and central suction tubing 613 a continual running of fluid in and out can be performed similar to wound irrigation. In some implementations, flow can be titrated to allow for correct flow rates to allow for maxim dialysis while limiting waste (for example the flow can be controlled by an EVR such as EVR 107). In some implementations, an additional air bladder (not shown) inside the bladder 606 can be used to increase bladder volume in the expanded state without needing to fill the entire bladder with fluid to reduce weight of the bladder. In some implementations, a plurality of columns or bumps on the outside surface of the air bladder and/or the inside surface of the bladder 606 are used to ensure there is a space for fluid flow between the two bladders. In such cases, two tubing systems would be required, the first tubing system to provide medication diffusion and/or suction from the external bladder 606 (e.g., peripheral tubing 612 and central suction tubing 613) and the second tubing system to provide air/fluid to the internal air bladder to increase volume of bladder 606 without needing to fill the entire bladder with fluid or medication.

In some implementations, the peripheral tubing 612 and/or the central suction tubing 613 has multiple lumens to allow for multiple functions, including one or more of providing air flow, providing negative pressure, providing irrigation, and providing medicaments and therapeutic substances. Additionally, in some implementations, the peripheral tubing 612 and/or the central suction tubing 613 includes wires in the lumen or out of the lumen to power sensors, lights or other devices inside the dressing. In some implementations, the lumens of the peripheral tubing 612 and/or the central suction tubing 613 are coated with therapeutics to deliver to the wound surface or to create chemical reactions, for example to identify the presence of particular cells or bacteria in the wound by fluorescence or visual observation. In an example, black light can be used to determine if bacteria is present in the wound bed or in exudate removed from the wound bed. In some implementations, chemical reactions can be created inside the lumen of the outflow tubing 604 to show the presence or absence of bacteria or specific cell types.

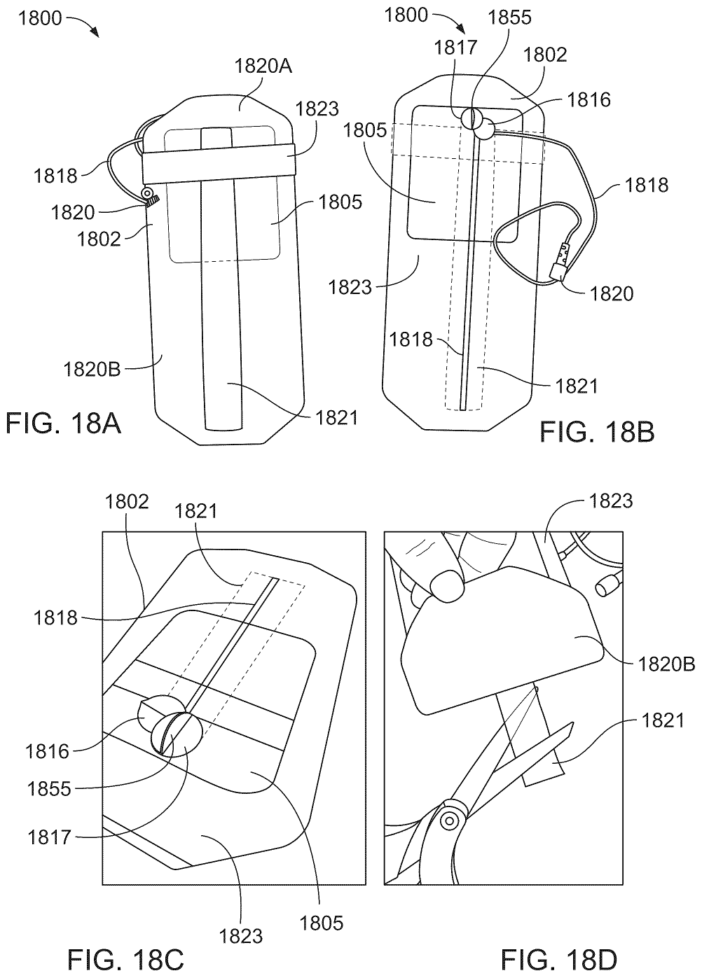

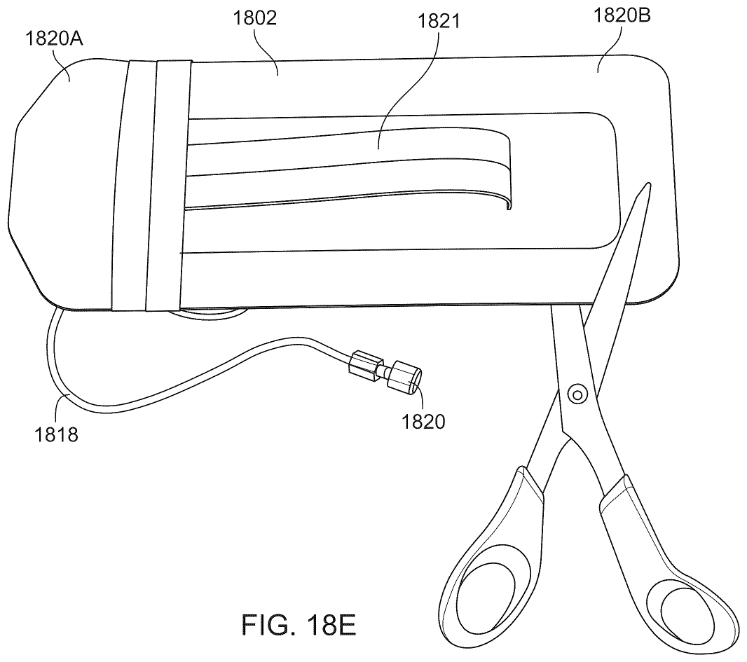

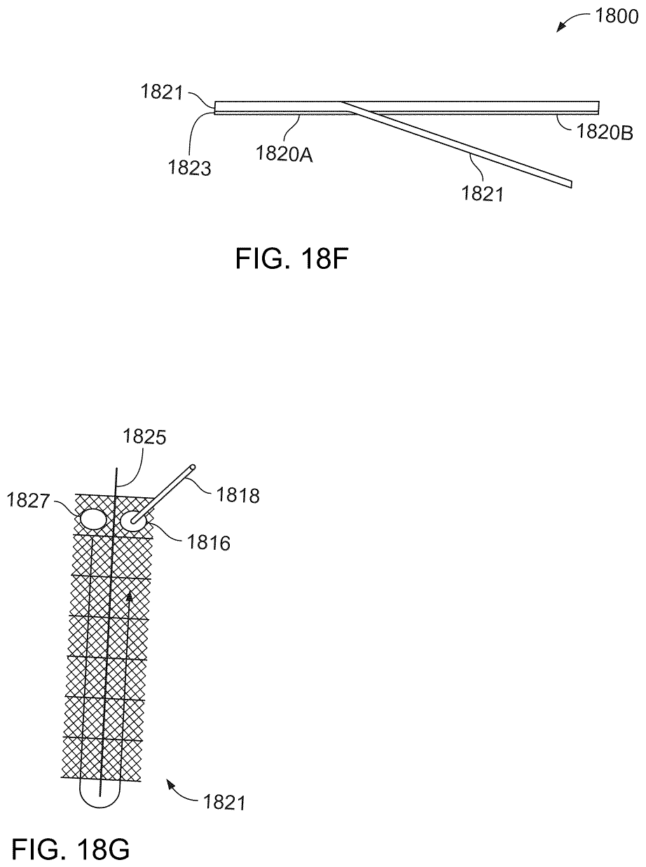

In some implementations, an alternative to central suction tubing 613 that allows air to be run across the top of the wound can include separating the wound contact material into two or more passageways using at least one dividing wall. At least one of the passageways can be used for air inflow while at least one of the other passageways can be used for air suction from the wound. Both the air inflow and air suction ports can be offset to one side of the wound contact material. This enables incoming air to travel the length of the wound contact material, around the dividing wall, and back across the wound until reaching outflow tubing 604. Such implementations are further illustrated and described with FIG. 18G.

In some implementations, the lumens of the peripheral tubing 612, central suction tubing 613, outflow tubing 604 or inflow tubing 602 include areas that are collapsible when not filled to prevent back flow. In such implementations, if there is not positive pressure keeping the lumens open, the lumens will collapse to limit inflow or backflow. Additionally, in some implementations, the lumens of the peripheral tubing 612, central suction tubing 613, outflow tubing 604 or inflow tubing 602 include one-way valves that prevent or reduce backflow of substrates or exudates within the lumen. For example, the one-way valves can be placed within the central suction tubing 613 and outflow tubing 604 so that once exudate is removed from the peripheral dressing 614 it cannot flow back into the wound bed. It is important to prevent backflow of the exudate into the wound bed, especially when the bladder 606 is coupled to a sequential compression type device that would pump fluid out of the peripheral dressing 614 and into the wound if fluid back-flowed into the peripheral dressing 614.

In some implementations, the flow of irrigation can be reversed within the peripheral dressing 614. Typical flow allows for central suction and peripheral deposition of irrigation fluid and therapeutic substances on the wound edges. In some implementations, the flow pattern can be reversed to allow peripheral suction, if required. In some implementations, the flow pattern can be reversed while the peripheral dressing 614 and bladder 606 is positioned in the wound and can be switched as needed by switching the tubing connections at the pump/suction source. In such implementations, the pump source is purposefully reversed in order to improve suction. In some implementations, suction is provided through both sets of inflow tubing 602 and outflow tubing 604 when irrigation is not being provided. In other words, suction is provided through both tubing systems to maximize exudate removal, so that suction would be performed at the center (i.e., at central suction tubing 613) as well as at the periphery at the ends of the radial tubing (i.e., at peripheral tubing 612). When irrigation is then desired, the central suction tubing 613 or the peripheral tubing 612 can be used to infuse fluid or whatever is desired to be placed either through the central part or the periphery.

In some implementations, a bladder that creates additional sequential and low pressure positive pressure can be incorporated into the incisional dressing. Similar in concept to sequential compression dressings used in lower extremities to avoid blood clots, the sequential bladder can be used to reduce swelling and venostasis and improve blood flow at the wound site. The inflation can be programed and automated in some implementations. The vacuum pump can be designed to provide inflation of the bladder in some implementations. The sequencing of the inflation and deflation, or sustained inflation or deflation, can be programed and automated in some implementations, and/or customized or preset in a number of programs.

The bladder (for example bladder 506 in FIG. 5 or bladder 606 in FIG. 6) can have a variety of shapes in an expanded state. The shapes can be determined to fit the type of wound and purpose of the bladder. FIGS. 7A-E illustrate exemplary shapes of an inflatable bladder for use in a wound bed. FIG. 7A shows a bladder 705 formed as a hemisphere with attached tubing 702. FIG. 7B shows a bladder 707 formed as a sphere with attached tubing 702. FIG. 7C shows a bladder 709 formed as a cone with tubing 702 attached at a bottom surface of the cone. FIG. 7D shows a bladder 711 formed as a column with attached tubing 702. FIG. 7E shows a bladder 713 formed as a cone with tubing 702 attached to a pointed tip of the cone. In each case, the bladder can be formed to have these shapes, or can be cut or otherwise specifically formed to match the wound bed. In some implementations, multiple dressings and/or bladders can be stacked to achieve an appropriate size or depth to match a wound bed.

In some implementations, if a crater-type wound needs to be covered, pie-shaped wedges are removed from one or both layers of a bladder and dressing to allow the bladder/dressing to be folded into a bowl-shaped formation. Additionally, in some implementations, bladder molds can be designed to match certain wound types or depths. In some implementations, the bladder is formed with a bowl shape to fit a wound bed.

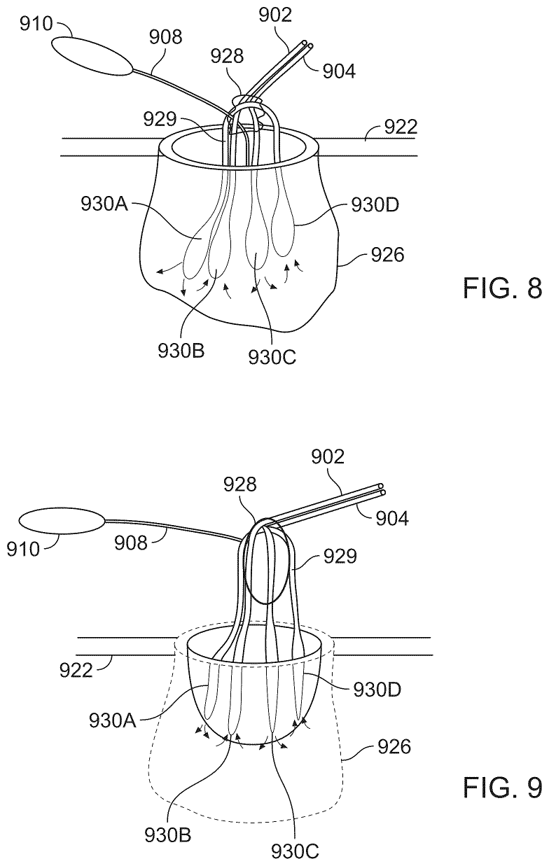

FIGS. 8 and 9 illustrate an exemplary NPWT dressing system including multiple extensions 930A-D in a first state (FIG. 8) and in a second state (FIG. 9). The dressing is formed from multiple dressings 930A-D each positioned at the end of an elongate arm 929. A dressing that includes multiple dressings 930A-D can be more easily adjusted to fit into multiple cavities or craggy cavities of a wound without allowing for dead space in the wound that could result in formation of an abscess. In other words, the multiple dressings 930A-D can be adjusted to more fully fill the space of certain types of wounds than a single wound dressing could. The dressing further includes an inflatable bladder 928 positioned where the elongate arms 929 join and used to expand a wound 926 to accept the dressings 930A-D and to slowly remove the dressings 930A-D from the wound. The dressing includes inflow tubing 902 and outflow tubing 904, and inflation pump 910 and inflation tubing 908 in fluid communication with the bladder 928. The dressing (termed an “octopus dressing”) provides a system for managing a cavity or tunnel wound 926. Because the dressings 930A-D are movable and flexible on the elongate arms 929, the dressings 930A-D can be pushed into deep cavity or tunnel wounds 926. In some implementations, dressings can be severed from the group of dressings 930A-D by cutting through the elongate arms 929 so that the dressings 930A-D are sized to fit the wound. In some implementations, the dressings 930A-D are inserted into the wound 926 while the bladder 928 is in a deflated state. In some implementations, the dressings 930A-D are inserted into the wound 926 while the bladder 928 is in an inflated state to expand the wound 926 or the skin surrounding the wound 926. Pump 910, inflation tubing 908, inflow tubing 902 and outflow tubing 904 remain outside of the wound 926 and external to the skin layer 922. Within the wound, the dressings 930A-D provide inflow to supply irrigation or therapeutic substances, and discussed above, and provide outflow through suction.

The construction of the dressings 930A-D allow the dressings 930A-D to be inserted into a wound 926 and then slowly removed from the wound 926. As the wound 926 heals, the dressings 930A-D and elongate arms 929 can be extracted from the wound 926 incrementally. The wound 929 will heal from the peripheral walls, so that the wound 926 becomes shallower and smaller as it heals. In some implementations, the removal can be performed through the use of inflatable bladder 928 that pulls the dressings 930A-D out as the bladder 928 inflates. For example, the dressings 930A-D can be placed in at least partial contact with a bottom surface of the wound 926 before being removed using the inflatable bladder 928. Bladder 928 can be inflated which causes it to stand up and by extension pull the dressings 930A-D out of the wound 926. In some implementations, the bladder 928 can be used to expand the wound 926. In some implementations, the bladder 928 can be sequentially inflated and deflated to assist in fluid elution. In some implementations, the bladder 928 can be deflated as the wound heals from inside out.

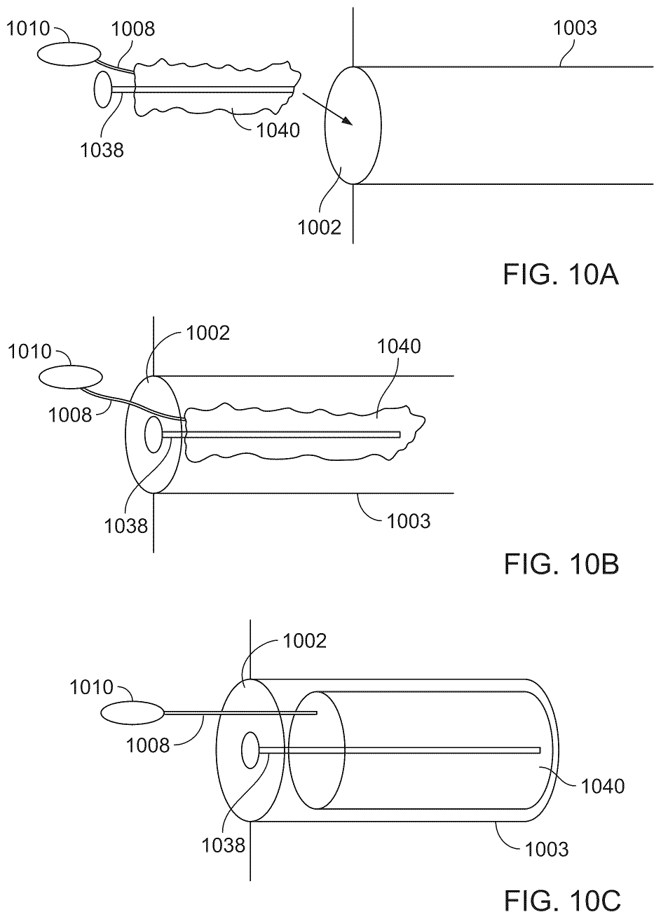

FIGS. 10A-C illustrate views of an exemplary inflatable bladder 1040 during insertion into a tunnel wound 1003 (sometimes also referred to as an incisional wound or tunneling wound). FIGS. 10A-C shows an inflatable bladder 1040 coupled to an inflation pump 1010 by tubing 1008. The inflatable bladder includes an insertion rod 1038 inserted into a central cavity of the inflatable bladder 1040. In FIG. 10A, inflatable bladder 1040 is in a deflated state. Inflatable bladder 1040 is inserted into a tunnel wound 10003 through an opening 1002 in a patient's skin while the inflatable bladder 1040 is in the deflated state. The insertion rod 1038 allows the inflatable bladder 1040 to be pushed into the cavity despite its lack of structure in the deflated state. Using the insertion rod 1038, the inflatable bladder can be deployed deep within the tunnel wound 1003.

In FIG. 10B, the inflatable bladder 1040 is fully inserted into the tunnel wound 1003. While within the tunnel wound 1003, the inflatable bladder 1040 remains connected by tubing 1008 to the inflation pump 1010 located outside of the opening 1002 of the tunnel wound 1003.

In some implementations, the inflatable bladder 1040 is translucent or transparent. In some implementations, the inflatable bladder 1040 includes a light or a camera within the inflatable bladder 1040 to allow medical providers to inspect the tunnel wound 1003. In some implementations, the inflatable bladder 1040 includes a port for insertion of a scope (e.g., arthroscope or endoscope). In some implementations, the inflatable bladder 1040 includes an array of tubing for providing irrigation and suction to create a negative pressure dressing. In some implementations, the inflatable bladder 1040 delivers irrigation or other medications deep to the tunneling wound. In some implementations, the inflatable bladder 1040 the tubing for irrigation and/or suction is functional or structural in nature.

In FIG. 10C, the inflatable bladder 1040 is in an inflated state in which it is filled with liquid or gas by the inflation pump 1010. In some implementations, the inflatable bladder 1040 is filled with air, water or other materials. In some implementations, the inflatable bladder 1040 is cooled or heated to provide specific effects. For example, the inflatable bladder 1040 can be cooled or heated to provide cold or hot treatment to the wound bed, for therapeutic or pain relief purposes. As another example, the inflatable bladder 1040 can be cooled or heated to produce a particular effect to a coating of the inflatable bladder or a covering of the inflatable bladder. In some implementations, the inflatable bladder 1040 is designed to allow osmotic transfer of chemicals into or out of the internal bladder contents and into the tunnel wound 1003 to allow for extended medication delivery. In some implementations, the contents of the inflatable bladder 1040 is cycled or exchanged.

In some implementations, the inflatable bladder 1040 is inflated so as to contact the edges of the tunnel wound 1003. The inflatable bladder 1040 reduces the premature closing of the tunnel wound 1003. In some implementations, the inflation pump 1010 is a pneumatic pump that is used to provide continuous pressure to the inflatable bladder 1040. In some implementations, the inflatable bladder 1040 is limited in the amount of pressure it can hold to prevent overinflation. In some implementations, the expandable bladder 1040 includes a drainage passageway to prevent premature closing by allowing drainage from deep within the tunnel wound 1003 to flow through the inflatable bladder 1040 and out the opening 1002 of the tunnel wound 1003. In some implementations, the insertion rod 1038 remains in the inflatable bladder 1040 while the inflatable bladder 1040 is positioned in the tunnel wound 1003.

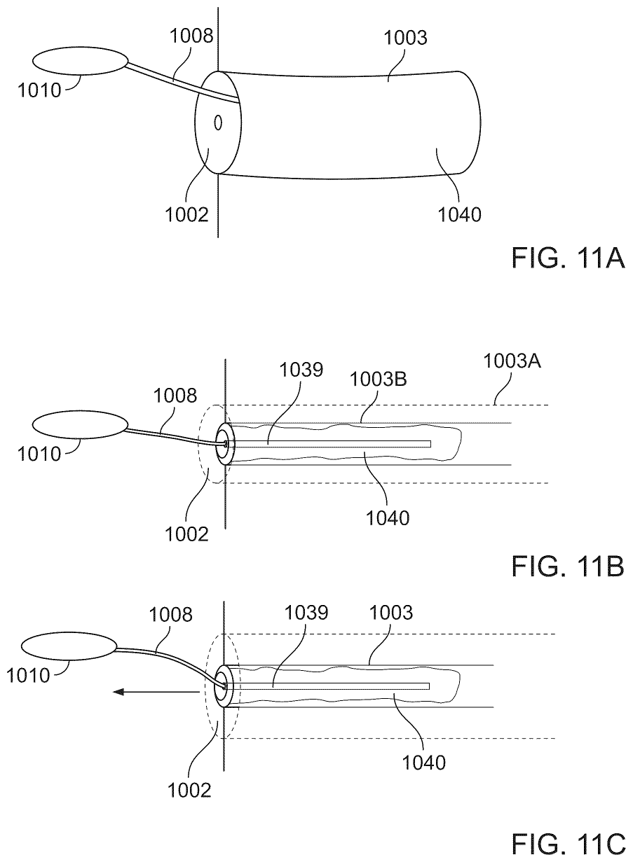

In some implementations, the inflatable bladder 1040 is deflated over time to allow the tunnel wound 1003 to heal from inside out. FIGS. 11A-C illustrate views of inflatable bladder 1040 during healing of the tunnel wound 1003 and extraction of the bladder from the tunnel wound 1003. FIG. 11A shows the inflatable bladder 1040 inserted into the tunnel wound 1003 and inflated to fille the space within the tunnel wound 1003. Insertion rod 1038 is not present in FIGS. 11A-C, though in some implementations the insertion rod 1038 remains in the inflatable bladder 1040 for the duration of the use of the inflatable bladder 1040 within the tunnel wound 1003. In FIG. 11B, the tunnel wound 1003 shown in FIG. 11A has healed from the outside toward a center of the wound so that the tunnel wound 1003B has a smaller diameter than the original diameter of the tunnel wound 1003B. The inflatable bladder 1040 has been deflated using inflation pump 1010 and tubing 1008 to fit the smaller diameter of the tunnel wound 1003B. In some implementations, a length of the inflatable bladder 1040 (i.e., the dimension of the inflatable bladder extending into the wound cavity from the opening 1002) is also reduced when the inflatable bladder 1040 is deflated to allow the wound to heal. In some implementations, medicament is provided to the tunnel wound 1003B through a channel 1039 (illustrated in FIGS. 11B-C) in the bladder 1040. In FIG. 11C, the inflatable bladder 1040 is removed from the tunnel wound 1003 to allow the tunnel wound 1003 to close fully. In some implementations, the inflatable bladder 1040 is removed incrementally from the tunnel wound 1003 to allow the tunnel wound 1003 to close at the very deepest location before further removing the inflatable bladder 1040. Incremental removal of the inflatable bladder 1040 decreases creation of abscesses and infection within the depths of the wound during healing. In some implementations, the inflatable bladder 1040 is fully removed once the wound has healed around the inflatable bladder 1040 when the inflatable bladder 1040 is in the fully deflated state. In some implementations, the inflatable bladder 1040 is removed once the wound has healed around the inflatable bladder 1040 when the inflatable bladder 1040 is in a semi-inflated state. In some implementations, the inflatable bladder 1040 includes a pressure sensor on its surface to detect whether the tissue in the tunnel wound 1003 has healed around the inflatable bladder 1040. In some implementations, the inflatable bladder 1040 is incrementally removed from the tunnel wound 1003 by the insertion rod 1038 (not shown in FIGS. 11A-C) to allow the tunnel wound 1003 to heal from inside out. In some implementations, the inflatable bladder 1040 is removed without use of an insertion rod.

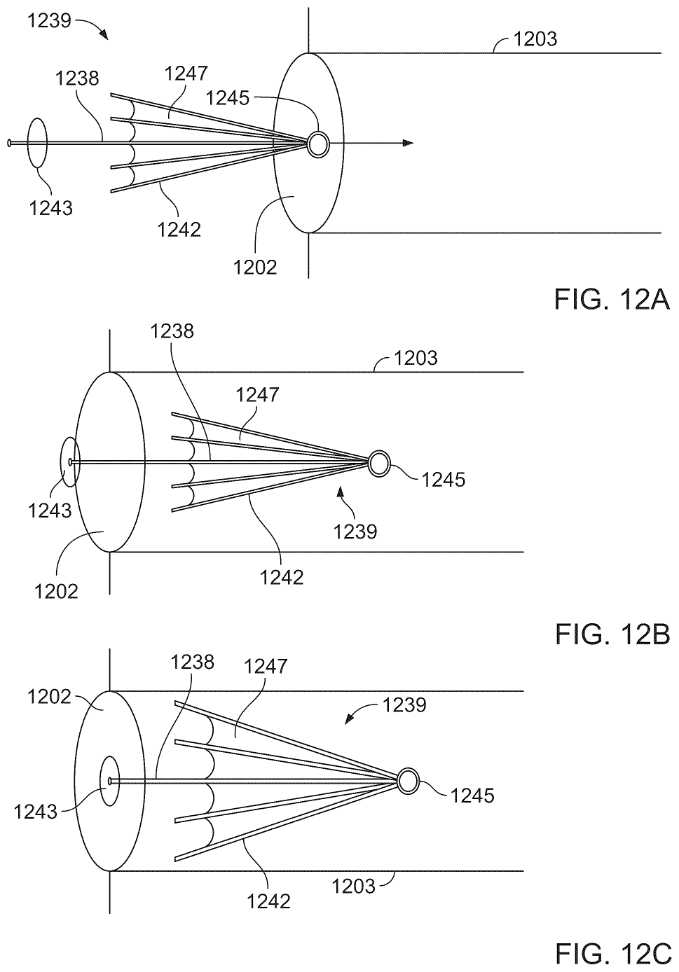

FIGS. 12A-C illustrate views of an exemplary expandable dressing 1239 during insertion into a tunnel wound 1203. The expandable dressing 1239 includes central rod 1238 with radiating ribs 1242 connected at distal end 1245 that can be expanded like an umbrella using an expansion toggle 1243. Dressing material 1247 connects each of the radiating ribs 1242. In some implementations, the central rod 1238 is constructed like an umbrella shaft with an expansion toggle 1243 to open the radiating ribs 1242. The expansion toggle 1243 can include simple open/closed positions or can includes preset intervals, a graded ratchet, or crank system to enable a medical provider to expand the radiating ribs 1242 to a desired position. The expandable dressing 1239 can be expanded within the tunnel wound 1203 to keep the wound open and can also be collapsed in a controlled fashion as the wound heals.

FIG. 12A shows the expandable dressing 1239 in a collapsed state with radiating ribs 1242 held close to the central rod 1238. Although the radiating ribs 1242 are shown extending outwards from central rod 1238 in FIGS. 12A and 12B for clarity, in some implementations, the radiating ribs 1242 can be held against central rod 1238 in the collapsed state. In the collapsed state, the expandable dressing 1239 is inserted into the opening 1202 of the tunnel wound 1203 and into a position within the tunnel wound 1203, as illustrated in FIG. 12B.

In FIG. 12C, the expansion toggle 1243 is used to expand the radiating ribs 1242 outward from the central rod 1238. In some implementations, the radiating ribs 1242 are expanded away from the central rod 1238 until they are in contact with the interior walls of the tunnel wound 1203. In some implementations, the central rod 1238 remains in the wound once the dressing is in place (as illustrated in FIGS. 12A-C). In other implementations, the central rod 1238 is removed once the radiating ribs 1242 have been expanded within the tunnel wound 1203.

The dressing material 1247 between the radiating ribs 1242 can be formed from a polymer to discourage tissue ingrowth. In some implementations, the dressing material 1247 is transparent or translucent. In some implementations, the dressing material 1247 includes suction and irrigation elements attached to or formed within the dressing material 1247. In some implementations, the dressing material 1247 is formed from perforated polymer sheets. In some implementations, the central rod 1238 and the radiating ribs 1242 house irrigation and suction tubing. In some implementations, the central rod 1238 and the radiating ribs 1242 include lights or pressure sensors for enabling visual inspection of the wound or determining healing and regrowth of the wound or position of the radiating ribs 1242 relative to sides of the tunnel wound 1203. For example, the expandable dressing 1249 can be a standard negative pressure dressing. In some implementations, the dressing material 1247 is stretchable (like the material of a balloon) or is inelastic (like the material of an IV bag). In some implementations, the dressing material 1247, radiating ribs 1242 and/or central rod 1238 are coated to provide healing and antimicrobial effects. The coating can include biological factors or skin supplements. In some implementations, the expandable dressing 1249 is used as a tissue expander to stretch tissue.

In some implementations, an incisional dressing (e.g., the dressings depicted in FIGS. 4-12) includes one or more shock absorbing components to protect the wound from pressure points within the dressing. In some implementations, the shock absorbing components are formed as additional padding or as gel additions to the dressing. In some implementations, the shock absorbing components are formed from silicone. In some implementations, the shock absorbing components can extend from each side of the dressing away from the wound so as to maintain visualization of the wound through the dressing. In some implementations, the shock absorbing components are positioned above a sealing layer of the dressing. Additionally or alternatively, the shock absorbing components are positioned below the sealing layer of the dressing. In some implementations, the shock absorbers are formed as air bladders that allow for compression of the tissue and/or sequential inflation/deflation of the bladders to promote increased blood flow.

In some implementations, an incisional dressing (e.g., the dressings depicted in FIGS. 4-12) includes absorbent material to promote fluid retention, where the absorbent material is sequestered so as not to allow retrograde flow of fluid toward the wound surface. In some implementations, a membrane allowing for unidirectional flow, one or more valves, or absorbent material positioned a distance away from the wound surface are provided to prevent retrograde flow from the absorbent material toward the wound surface to reduce or inhibit fluid from remaining on the wound surface.

### VIII. Customizable Incisional Dressing

After a major operation (e.g., spinal fusion or hip arthroplasty), an incisional surgical wound would be closed with sutures or other material, but these types of wounds can seep. Wound dressings are conventionally applied in these settings to control the seepage, and when negative pressure wound therapy dressings are used, the enhanced blood flow to the skin surface under the influence of negative pressure can aid in healing. Vacuum dressings for use with closed incisions are clinically efficacious. However, implementing a negative pressure wound therapy system in out-patient settings can be difficult.

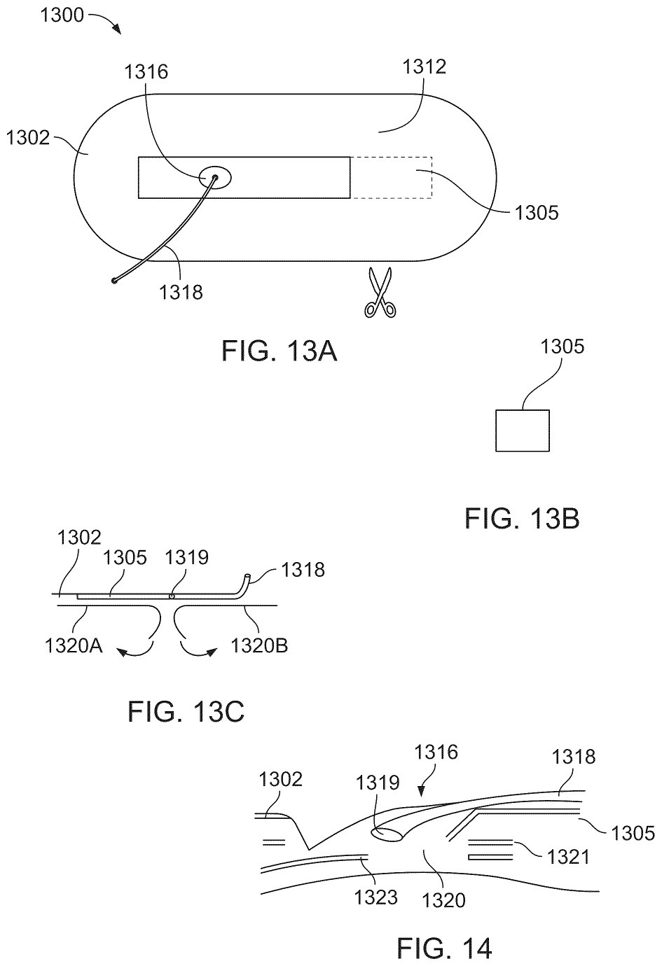

A disposable negative pressure wound therapy system composed of a customizable and disposable dressing, tubing and a portable disposable pump can be used to provide negative pressure therapies to closed incisional wounds in the out-patient setting. Disposable negative pressure wound therapy system disposable systems can be maintained with one dressing placement in a clinical setting (e.g., hospital or outpatient clinic) while allowing for therapeutic delivery at home. FIGS. 13-22 describe exemplary features of disposable negative pressure wound therapy systems. Although some features are described with regard to a particular figure, the features described in FIGS. 13-22 can be combined in a single disposable negative pressure wound therapy system. Additionally, the disposable negative pressure wound therapy systems described with regard to FIGS. 13-22 can be formed from a transparent or translucent polymeric material as described above with regard to FIG. 1 and can include the features and benefits described with regard to the FIG. 1.

FIGS. 13A-C illustrate views of an exemplary customizable NPWT dressing 1300 for use in out-patient settings. The dressing 1300 includes wound contact layer 1305, sealing layer 1302, and port 1316 coupled to tubing 1318. The port 1316 is offset from a center of the dressing 1300 to allow for added customizability when a medical professional cuts the dressing 1300 to size. Tubing 1318 provides suction for negative pressure therapy to the wound.

The dressing 1300 has a unified construction (i.e., all pieces are united in one product) but remains universally customizable to the length of the wound. The sealing layer 1302 is formed as a clear sticker with peelable backing 1320A-B (shown in FIG. 13C). The sealing layer 1302 extends over the wound contact layer 1305 but is connected to the wound contact layer 1305 at least at the or around the port 1316. By coupling the sealing layer 1302 to the wound contact layer 1305 only at a portion of the wound contact layer 1302, the two layers can be separately customized by cutting to a size of the wound, and still applied to the wound together so that the sealing layer 1302 covers the wound contact layer 1305 and so that tubing 1318 extends through the sealing layer 1302 into the wound contact layer 1305 without the need for additional assembly by the medical professional.

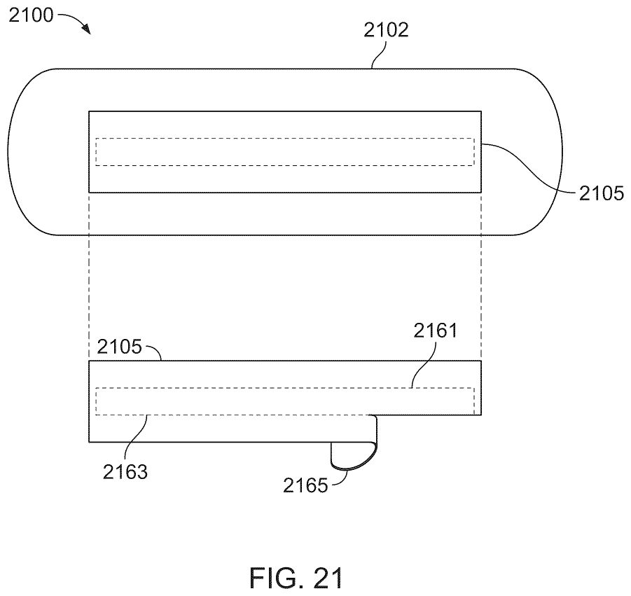

For example, as illustrated in FIGS. 13A-B, the wound contact layer 1305 can be cut to a size of the wound so that the sealing layer 1302 completely surrounds the wound contact layer 1305. In some implementations, the dressing 1300 can be cut to any length. In other implementations, the dressing 1300 includes perforations or guides for cutting to a number of lengths (see e.g., FIG. 21). In some implementations, the remaining portion of dressing 1300 is used to cover a closed incisional wound. In other implementations, a distal end piece of sealing layer 1302 can include markings illustrating where the wound contact layer 1305 underlays the sealing layer 1302. The specific construction of this dressing 1300, in which a portion of the wound contact layer 1305 is separated from the adhesive sealing layer 1302, allows the adhesive and wound covering elements to be cut to the proper length to match the wound without requiring additional add-on adhesive.

In conventional wound coverings, the wound covering and adhesive drape are typically non-unified (so that the medical provider needs to assemble the dressing piecemeal) or are unified such that the adhesive drape and the wound covering are mated and inseparable. When an inseparably mated adhesive drape and wound covering are cut to length, the adhesive drape is the same length as the wound covering, requiring an additional adhesive drape to be added as an “end-cap” which can cause a leak site that compromises the dressing. Coupling the adhesive sealing layer 1302 and the wound contact layer 1305 as described above with regard to FIGS. 13A-C allows each layer to be separately cut to size while maintaining the layers relative to one another. FIG. 13C illustrates the peel-off backing paper that separate the wound contact layer 1305 from the sealing layer 1302, so the wound contact layer 1305 can be cut to incision length, while the sealing layer 1302 can be cut to be longer (for example 1-2 cm longer), if necessary, to ensure a good seal of the sealing layer 1305 to the patient's skin surrounding the wound contact layer 1305.

The wound contact layer 1305 can be formed from polymeric materials that discourage tissue ingrowth, as described above. The wound contact layer 1305 can further by a transparent or translucent material that allows for direct observation of the wound during the period of treatment. Direct observation of the wound enables earlier detection of wounds that are not healing well or are infected so that treatment (or transport to a facility to receive treatment) can be initiated more quickly. Direct observation of the wound also allows wound healing progress to be observed without removal of the dressing, so that the dressing can be worn by the patient for a longer duration.

The port 1316 connecting internal tubing in the wound contact layer 1305 to an external pump (not shown) delivers negative pressure to the dressing. In some cases, a port could become a pressure spot and injure underlying tissue. To prevent the port 1316 from acting as a pressure spot, the port 1316 is formed with a low profile connection between the unified dressing 1300 and the tubing 1318. For example, FIG. 14 illustrates a cross-sectional view of an exemplary low-profile port 1316 for use in dressing 1300.

In FIG. 14, tubing 1318 is connected to wound contact layer 1305 at an angle so that the body of the tubing 1318 lays parallel to the top surface of the wound contact layer 1305 and sealing layer 1302 until it enters the wound contact layer 1305. The opening end 1319 of the tubing 1318 may extend into a space 1320 formed in the wound contact layer 1305 as shown where multiple suction tubes 1323, 1321 open into the space 1320. Applying suction through the tubing 1318 provides suction to the multiple suction tubes 1323, 1321 so that suction can be applied to the wound surface. The angled entry of the tubing 1318 into the space 1320 decreases the risk that the port 1316 will form a pressure point compared to tubing entering the space 1320 at a right angle, and the tubing 1318 lying along the sealing layer 1302 further decreases the risk. Tubing 1318 can be taped down to the top of the sealing layer 1302 to hold it in a desired position and prevent or reduce kinking of the tubing 1318. In some implementations, edges of the sealing layer 1302 and wound contact layer 1305 are very thin and tapered to avoid pressure concentration points or discomfort. In some implementations, one or more portions of the dressing 1300 can be built up in order to allow for cushioning in order to prevent pressure ulcers.

In some implementations, the low-profile port 1316 at which tubing 1318 is connected is centered in a width of the dressing 1300. In some implementations, the low-profile port 1316 at which tubing 1318 is connected is positioned at an end of the dressing 1300. In some implementations, the connection between the tubing 1318 and wound contact layer 1305 is generally orthogonal. In some implementations, the connection between the tubing 1318 and wound contact layer 1305 is in the plane of the dressing to reduce pressure concentration at the connection. In some implementations, the wound contact layer 1305 has a corrugated (undulating) surface facing the wound and a smooth, flat side opposite the wound. The smooth, flat, non-wound-facing side of the wound contact layer 1305 would be the side at which the sealing layer 1302 adheres and secures the wound contact layer 1305. The undulating side of the wound contact layer 1305 would produce small air pockets or passageways to convey negative pressure over the length of the dressing 1300. In some implementations, the wound contact layer 1305 is solid. In other implementations, the wound contact layer 1305 has an “air chamber” construction with a very thin central chamber including risers within the central chamber to keep the chamber open when negative pressure or external compressive pressure is applied. The air chamber or passageway can allow air flow and exudate to travel towards the connection between the suction tubing and the dressing. In the chambered embodiment, small perforations in the wound contact layer 1305 face the wound to convey the negative pressure through the wound contact layer 1305 to the wound surface. In both solid and chambered embodiments, in some implementations, the connection point between the wound contact layer 1305 and tubing 1318 is a melded joint in which the tubing 1318 merges with the wound contact layer 1305 as a single piece either extending from one end of the wound contact layer 1305 or positioned at a right angle (or other angles, for example, 45 degrees, 30 degrees, 60 degrees, or any other suitable angle) from the midpoint of the length of the wound contact layer 1305 in the plane of the dressing 1300. When the connection point is at the midpoint, the dressing can be shortened (e.g., cut to size) in either direction. When the connection point is at end of the wound contact layer 1305, the tubing 1318 is in line with the long axis of the incision, and with the long axis of the extremity or trunk in which the incision is formed, which allows for movement of the patient (e.g., walking) with the dressing 1300 in place.

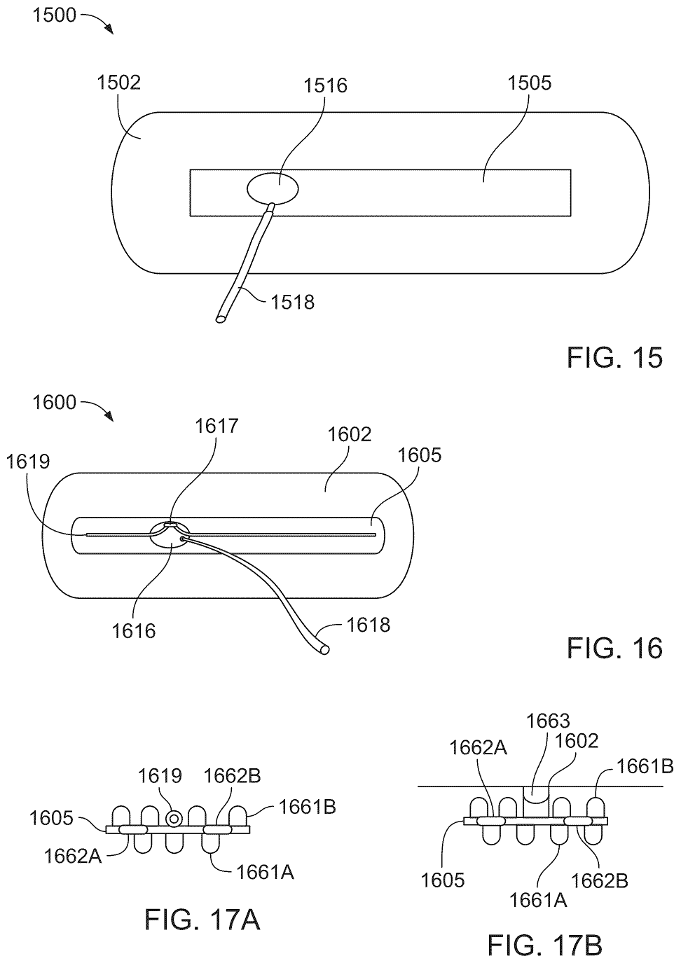

In some implementations, the dressing includes a central suction port formed as a channel through the sealing layer 1302 and into the wound contact layer 1305 for connection of tubing 1318 to the tubing of the wound contact layer 1305 without any elevation above the sealing layer 1302. For example, in FIG. 15, illustrates an exemplary incisional NPWT dressing 1500 including a central suction port 1516 and tubing 1518 for providing suction to the wound. In FIG. 15, sealing layer 1502 is formed so as to be unified with the wound contact layer 1505 at the central suction port 1516 but able to be separated for individual cutting to size of the sealing layer 1502 a wound contact layer 1505 at least at the end opposite the central suction port 1516, as described above. The dressing 1500 includes central suction port 1516 which is built into the dressing so as to have no elevation above the sealing layer 1502 so that no pressure point is formed by the central suction port 1516. The central suction port 1516 can be built into the polymer of the wound contact layer 1505 as it is molded and can be constructed within the normal height of the dressing.

The central suction port 1516 includes an angled opening into which tubing 1518 extends. In some implementations, tubing 1518 is pre-inserted into the angled opening during manufacture. In other implementations, tubing 1518 is intended to be inserted into the angled opening by a medical professional. The central suction port 1516 provides the angled opening through the sealing layer 1502 and into the wound contact layer 1505 as an air-tight channel through the sealing layer 1502. The angled opening for the tubing 1518 and central suction port 1516 that does not extend above the sealing layer 1505 can reduce the risk of the port acting as a pressure point and causing damage to underlying tissue.

In some cases, positioning a closed dressing over a healing wound can create a closed space in which healthy skin at the margins of the incision are kept in a prolonged high moisture state, which can lead to maceration and skin injury. Skin is intended to be a dry structure, and constant exposure to fluid can cause adverse conditions. To aid in keeping the skin dry, the dressing can be formed with a system for providing a controllable drying ambient air flow across the wound to allow outside air to pass over the wound surface. In some implementations, the air is first passed through a microbial filter (i.e. HEPA). Providing air flow over the wound helps to keep the skin dry at the margins of the incision.

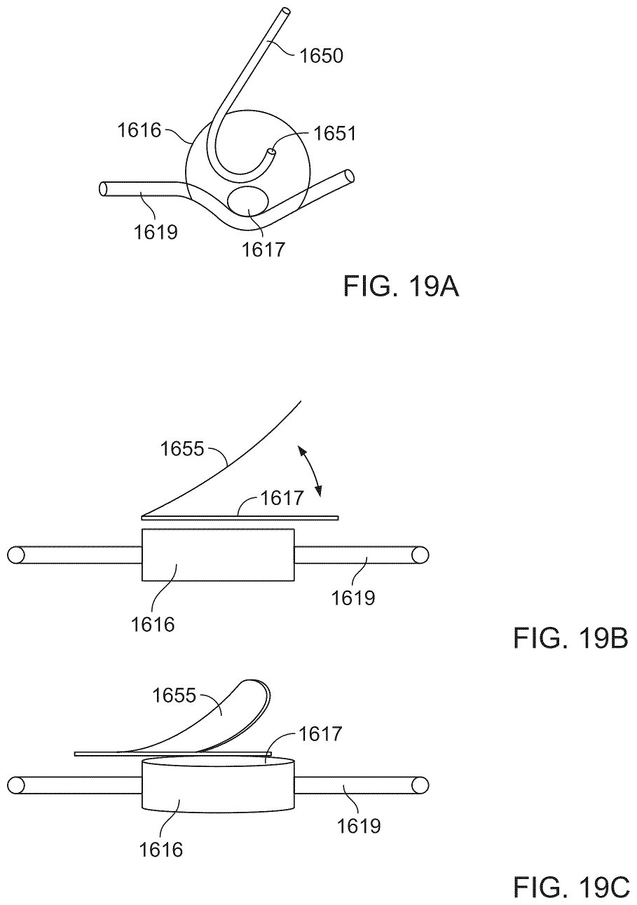

For example, FIG. 16 illustrates an exemplary incisional NPWT dressing 1600 including an opening 1617 for providing airflow to the wound. FIG. 16 illustrates dressing 1600 including sealing layer 1602, wound contact layer 1605, central suction port 1616, opening 1617, ad suction tubing 1618. The central suction port 1616 includes opening 1617 to access the atmosphere and includes a filter (for example a HEPA filter) to filter incoming air. In some implementations, the opening 1617 is able to be opened or closed with a resealable flap or by other means, including a twist valve, crank, pull string, or any other means for controlling a flow of air into the central suction port 1616. FIG. 19B illustrates a central suction port 1616 with opening 1617 covered by resealable flap 1655 in an open state. When the opening 1617 is uncovered by the resealable flap 1655, air is able to flow through the opening 1617 and into flow tubing 1619 formed within the wound contact layer 1605 (shown in FIG. 16). FIG. 19C illustrates the resealable flap 1655 in a partially closed state. In some implementations, a flow rate of air through the opening 1617 is controlled by a position of resealable flap 1655 (e.g., when the resealable flap 1655 is open, flow rate is at a maximum and flow rate is decreased as the resealable flap 1655 is moved toward a closed position).

In some implementations, the resealable flap 1655 is transparent or translucent. In some implementations, when air flow (or increased airflow) is desired, the resealable flap 1655 can be removed or pulled back (see FIG. 19B) and attached to a holding area (not shown) so that the opening 1617 and filter are exposed to air and allow flow through the opening 1617. In some implementations, when the flow is desired to be reduced or shut off, the resealable flap 1655 can be partially covered or completely covered to restrict flow (see FIG. 19C).

The central suction port 1616 is where the flow tubing 1619 that extends through the wound contact layer 1605 connects to the filter and allows flow of filtered air to the ends of the dressing 1600 and to a wound surface. In some implementations, the air flow from the flow tubing 1619 is directed over the suture line of the wound from the ends of the dressing 1600 back to the central suction port 1616. This allows for flow across the wound and sutures to dry the exudate instead of absorbing it and holding it on the wound edge (as in conventional dressings). Drying the exudate can reduce the risk of maceration and bacterial overgrowth.

In some implementations, the dressing 1600 is a single use and disposable system. In some implementations, the suction tubing 1618 of dressing 1600 is attached to a reusable or disposable negative pressure source (for example a pump). In some implementations, the negative pressure source is powered by batteries, or mechanical/manual power such as springs, rachets or other stored energy systems that do not require electrical power. In some implementations, a power source associated with the negative pressure source is a rechargeable battery or battery powered by solar energy. In some implementations, the pump that provides negative pressure (i.e., the negative pressure source) includes a collection cannister that is separate from the dressing 1600 and positioned away from the wound or a collection canister integrated into or on the wound contact layer 1605 and away from the wound surface. In other words, in some implementations the wound contact layer 1605 includes hyper-absorbent material on a side of the wound contact layer 1605 opposite from the wound-facing surface. The hyper-absorbent material absorbs exudate or other moisture from the wound and can reduce the occurrence of fluid retention at the wound surface.

In some implementations, to maintain clearness for direct wound observation through the dressing 1600, the hyper-absorbent material is formed as part of a canister/collection chamber external to the dressing 1600 or is added as absorbent material within suction tubing 1618 external to the dressing 1600 that traps and contains the fluid removed from the dressing 1600 before the fluid reaches the pump. In some implementations, this suction tubing 1618 is replaceable if excess exudate is encountered, while still preserving function of the pump and dressing 1600. In some implementations, the suction tubing 1618 includes hyper-absorbent material along the walls of the tubing. In some implementations, the hyper-absorbent material is formed as sponges, pads, hyper-absorbent balls, rings, or strips that are positioned in the inner lumen of the suction tubing 1618 to collect liquid or exudate that is suctioned from the wound. In some implementations, the suction tubing 1618 includes one-way valves at various locations to prevent retrograde flow back towards the wound surface. By removing exudate from the wound surface and collecting the exudate in hyper-absorbent materials away from the wound surface, bacterial overgrowth at the wound surface can be reduced relative to conventional dressings that include sponges within the dressing for soaking up exudate from the surface of the wound.

In implementations that use a collection canister, a hyper-absorbent material used to coagulate liquids in the OR is pre-loaded in a collection canister to solidify exudate as it is removed from the wound. The solidified exudate is then pumped out of the collection canister or emptied (e.g., a disposable bag or container can be removed from the collection canister) and the canister can continue to be used to collect exudate.

The disposable dressings described in FIGS. 13-22 can be used not only in closed wound management, but also can be used for providing covering and therapy to various low-exudate producing wounds. For example, smaller, chronic wounds that produce limited amounts of fluid can be treated with this disposable system such as peripheral vascular disease wounds, neuropathy (diabetic) wounds, or pressure ulcers. In some implementations, the dressings are formed from a thin, clear, polymeric wound contact layer that is positioned over low-exudate wounds without providing negative pressure therapy. In some implementations, where the wound extends deeper into the tissue, the dressing can be placed over the wound and the tubing can be connected to a disposable pump to provide negative pressure therapy. For treatment of low-exudate producing wounds, the disposable pump could have a collection canister (as described above) that is a single-use canister, a reusable canister, or a replaceable canister. In some implementations, the exudate is collected in hyper-absorbent material in the suction tubing, as described above, to collect the limited amount of fluid that is produced by these types of wounds. All of the features of the previously described closed wound dressing system would be applicable to dressings designed for use with low-exudate wounds.

With the use of low-resistance and non-compressible dressings, lower pressured devices may be used to provide negative pressures to the wound surface. For example, for use with either closed wounds or low-exudate wounds, in some implementations, the pump is a Jackson Pratt pump/canister (or similar) in which negative pressure is created using the collection canister as the negative pressure space using springs that expand the collection canister volume. In some implementations, the pump operates in two modes including (1) a maintenance stage with low flow creating a constant negative pressure and (2) a “sump-pump” type setting in which manual or automatic pumping occurs to pump/pull air or fluid across the wound to be collected in the canister and expelled from the collection canister through a series of one-way valves to pull the fluid away from the wound and out of the system.

In some implementations, a series of springs having different tensions and being arranged in different orientations can be constructed to create sustained and relatively constant negative pressure over extended periods of time. Springs have variable strength based on how much they are compressed: with larger compression, a spring exerts a stronger force as compared to a spring under minimal compression. In some implementations, springs of different location, strength, stiffness and orientation can be designed to maintain a smoother suction value as negative pressure is applied and a collection canister expands or fills. In some implementations, a spring-based pump system is used with a series of canisters so that springs with different levels of compression and suction and/or different orientations provide sustained and stable suction levels over an extended period of time. In some implementations, non-electric pumps can be managed using small electronic sensors or motors with very small electrical needs (e.g., pumps using chargeable batteries or other sources of power). In some implementations, chemical reactions that can be reversed and repeated such as reactions that cause heat or cold or expansion of gases or fluids can be utilized to create sustained low-powered suction sources.

Spring-loaded suction canisters are generally not used with conventional sponge-based NPWT dressings, because such dressings are filled with air and spring-loaded suction canisters use up potential suction application by removing the air from the sponge. In contrast, non-compressible dressings as described herein do not fill with air and a spring-loaded suction canister is able to provide immediate or near immediate transmission of negative pressure.

In some implementations, such suction devices include one-way valves to provide a bilge function where the pump extracts fluid (exudate or irrigation or other fluids or gases) off the wound surface and expels it to the environment or collection source which can be emptied as needed. In some implementations, suction devices additionally or alternatively include one-way valves to provide steady application of negative pressure in a closed and sealed dressing.

As illustrated in FIG. 16, the flow tubing 1619 coupled to the opening 1617 can be formed as a single tube running down the middle of the wound contact layer 1605 much like a spine. In some implementations, the flow tubing 1619 is formed as a single tube running through the wound contact later 1605 offset from a middle. As described above, in some implementations the flow tubing 1619 is connected to a HEPA filter to allow filtered air flow over the incision to promote drying of a leaky wound. As also described above, in some implementations the opening 1617 or the filter itself is adjustable to monitor or control flow. In some implementations, a twist valve titrates air flow or a resealable tab or flap can control flow.

In some implementations, the flow tubing 1619 is formed as a spine running the full length of the wound contact layer 1605. In some implementations, the flow tubing 1619 can be cut with the rest of the wound contact layer 1605 so that the flow tubing 1619 delivers air or fluid to the ends of the dressing 1600 where the air or fluid is then sucked back across the wound/incision towards the central suction port 1616. For example, FIG. 17A illustrates a side views of an exemplary perforated wound contact layer 1605 with flow tubing 1619. In FIG. 17A, the flow tubing 1619 is formed on wound contact layer 1605 between a first perforation line 1662A and second perforation line 1662B. The wound contact layer 1605 in FIG. 17A also includes spacing structures 1661A on the wound-facing surface and spacing structures 1661B on a top surface opposite the wound-facing surface. The spacing structures 1661A can reduce tissue ingrowth into the wound contact layer 1605 and further provide passageways for fluid flow or suction between the wound surface and the wound contact layer 1605 and promote flow parallel to the wound surface. The spacing structures 1661B provide passageways between the wound contact layer 1605 and the sealing layer 1602 for additional fluid flow or suction or to create cushioning in the dressing. Spacing structures 1661A-B can be formed as walls, posts, bumps, or any other suitable projection from the wound contact layer 1605. For example, the spacing structures 1661A-B can be formed as any one or more of the structures depicted in FIGS. 20A-J.

FIG. 17B illustrates a variation of the flow tubing 1619, in which the wound contact layer 1605 includes a semi-circular projection 1663 that forms a passageway in combination with the sealing layer 1602 to form the flow tubing 1619.

In some implementations, the wound contact layer 1605 includes more than one tubing spine to provide irrigation, medicament or other gasses in addition to providing suction. In some implementations, additional gases such as oxygen or other gases are added to the airflow or attached to the HEPA filter by additional tubing to treat the wound surface with specified concentrations of specific gases that promote beneficial healing of the wound.

In some implementations, inflow through the disposable dressing 1600 can be constructed as an instillation-type inflow, in which suction is halted and an inflow substrate is inserted over the wound or suture line. For example, the inflow can include hydrogen peroxide or alcohol. In some implementations, the inflow chemicals or substrates are humidified or inserted into the dressing through the central flow pathways either with or without active suction. The medication introduced to the wound bed may be used to promote healing, cleanse the wound, promote tissue healing, or for another purpose. In some implementations, the therapeutic medication or other substance may be driven by negative pressure sucking the therapeutic across the wound. In some implementations, the medicament or other substance is positively pushed across the wound via gravity or positive pressure manually or via the pump. Alternatively, in some implementations, the filter can be treated with a chemical and allowed to be aerosolized over the wound/suture line. Additionally, in some implementations, an area similar to a port catheter injection port is formed in the dressing that allows for repeated injections into the flow tubing 1619 with resealing capabilities between or after injections. In some implementations, the areas formed from a material allowing for repeated injections into the flow tubing 1619 is positioned next to the HEPA filter or is separated by space. These areas can be formed from a material that provides a soft or sticky covering which is able to seal around the injection sites to allow for injection without loss of the seal for the system. Additionally, in some implementations, standard ports can be constructed that provide access to the flow tubing 1619 to insert or inject material into the dressing 1600.

In some implementations, the HEPA filter is treated with a powder that is sucked across the wound/incision as air passes through the system. In some implementations, liquid is placed on top of the filter to moisten or deliver vapor therapeutics to the incision. In some implementations, rubbing alcohol or hydrogen peroxide is placed in the filter to dispense isosorbide alcohol over the wound.

In some implementations, the dressing 1600 allows purified or filtered air flow to the wound bed that can be turned on/off or otherwise regulated to obtain the desired level of flow. In some implementations, this air flow can reduce negative pressure at the wound bed. In some implementations, the air flow is restricted through dense filters, small tubing, or adjustable rate devices to limit flow and allow for maintenance of the negative pressure gradient even with a smaller or weaker pump. This can allow the dressing 1600 (as well as dressings 1300, 1500, 1600, 1800, 2100, 2200) to be used in non-hospital or non-medical settings where access to a large suction pump or wall suction is not available. For example, the dressing 1600 can be used with a small pump by first responders, military medics, or EMTs in non-medical settings.

For example, in the setting of acute trauma or injury, a compressible dressing can be used initially to promote surface contact pressure in order to reduce or stop bleeding. In military or civilian wounds where blood loss is a concern, a compressive dressing with high negative pressure can be designed to promote pressure and stop bleeding. After the use of the initial compressive dressing, a second or additional non-compressive dressing (e.g., the incisional dressings described herein) to limit surface compression may be used once hemostasis is obtained. In some implementations, high-flow pathways within the non-compressive dressings may be utilized to deliver hemostatic agents to the wound. In other implementations, restricted-flow pathways within compressive or non-compressive dressings may be utilized to prevent removal of blood in order to limit blood loss. High negative pressure and restrictive pathways may be designed to serve the purpose of a temporary hemostatic dressing while avoiding circumferential tourniquets.

In some implementations, the central suction port 1616 is formed within the height of the wound contact layer 1605 (for example as described above with regard to FIG. 14). Additionally, FIG. 19A illustrates a central suction port 1616 with a low profile for providing suction through suction tubing 1650 and airflow through opening 1617. Central suction port 1616 includes a channel for receiving suction tubing 1650. Suction tubing 1650 enters central suction port 1616 at an angle and is further curved at entry into the central suction port 1616 so that opening 1651 faces away from opening 1617. Opening 1617 provides airflow to flow tubing 1619 that extends though the wound contact layer 1605 as described above. By positioning the opening 1651 of suction tubing 1650 to point away from opening 1617, air can flow through opening 1617 and through flow tubing 1619 to the edges of the wound contact layer 1605 before suction from opening 1651 of suction tubing 1650 directs the air to flow back toward the central suction port 1616. This provides air flow to the surface of the whole wound.

As described above, the features described with regard to FIGS. 13-17 can be combined into a disposable negative pressure wound therapy system. For example, FIGS. 18A-E illustrate a dressing 1800 including multiple features described above and steps of the method for customizing the dressing 1800 for application to a wound. FIG. 18A shows a back (wound-facing) side of dressing 1800, and FIG. 18B shows a front side of dressing 1800. Dressing 1800 is formed as a sealing layer 1802 that has a top (non-adhesive) side 1823 and an adhesive side covered by peelable backing 1820A-B having tab 1823, wound contact layer 1805, central suction port 1816 and suction tubing 1818, suction tubing connector 1820, opening 1817 with resealable flap 1855, and separating tab 1821. The central suction port 1816 is depicted as slightly offset from a midline of the dressing 1800. In some implementations, the central suction port 1816 is positioned at a midline. In other implementations, the central suction port 1816 is positioned further to one side of the dressing 1800. In some implementations, sealing layer 1802 extends longitudinally to a position beyond separating tab 1821 as illustrated (e.g., sealing layer 1802 covers separating tab 1821 when viewed from the front side of dressing 1800 in FIG. 18B). This allows the sealing layer 1802 to be customized to the length of an incision or wound, thereby providing patient-specific characteristics to the dressing 1800. In other implementations, separating tab 1821 may extend longitudinally to a position beyond sealing layer 1802. Particular implementations of sealing layer 1802 and separating tab 1821 may be used depending on the wound or other patient-specific characteristics.

The peelable backing 1820A covers the adhesive of the sealing layer 1802 and the wound contact layer 1805. The peelable backing 1820B covers only the adhesive of the sealing layer 1802, and extends between the sealing layer 1802 and the wound contact layer 1805, so that the wound contact layer 1805 can be separated from the sealing layer 1802 and both components can be separately cut to size so that the sealing layer 1805 can be cut to be larger/longer than the wound contact layer 1805 so that it extends beyond an end of the wound contact layer 1805 to provide a good seal on patient skin surrounding the wound. Separating tab 1821 aids a medical professional in separating the wound contact layer 1805 from the sealing layer 1802 and can also help to pull the peelable backing 1820B from the adhesive of the sealing layer when the dressing 1800 is in position over the wound.

The peelable backing 1820A-B can be gripped at the tab 1823 and pulled in opposite directions to expose the adhesive surface of the sealing layer 1802 (see FIG. 13C). Both sides of the peelable backing 1820A-B can be removed at the same time before positioning the dressing 1800, or alternatively, one side can be removed to expose a portion of the sealing layer 1802 so that the dressing 1800 can be positioned over the wound before the dressing 1800 is cut to size.

To customize and apply the dressing 1800, the peelable backing 1820A is removed. The dressing 1800 is then positioned over the wound or incision so that the wound contact layer 1805 is in contact with the wound. In some implementations, the wound contact layer 1805 is positioned so as to extend past the edge of the wound, for example a few mm past an edge of the wound. In some implementations, the wound contact layer 1805 is positioned so as to be just short of the edge of the wound. The adhesive backside of the sealing layer 1802 exposed by removal of peelable backing 1820A extends over the skin around the wound to secure the wound contact layer 1805 in position.

Next, the sealing layer 1802 for which the adhesive is still covered by peelable backing 1820B is folded away from the wound to expose the wound contact layer 1805 beneath. In some implementations, the separating tab 1821 is used to fold the sealing layer 1802 away from the wound contact layer 1805. The wound contact layer 1805 remains positioned over the wound and can be cut to a desired length. In some implementations, the desired length is longer than the wound, even with an end of the wound, or shorter than the wound. After the wound contact layer 1805 has been cut to the desired length, the peelable backing 1820B is removed to expose the adhesive backside of the sealing layer 1802. The adhesive backside of the sealing layer 1802 secures the wound contact layer 1805 in place by adhering to the skin surrounding the wound to create a sealed dressing 1800.

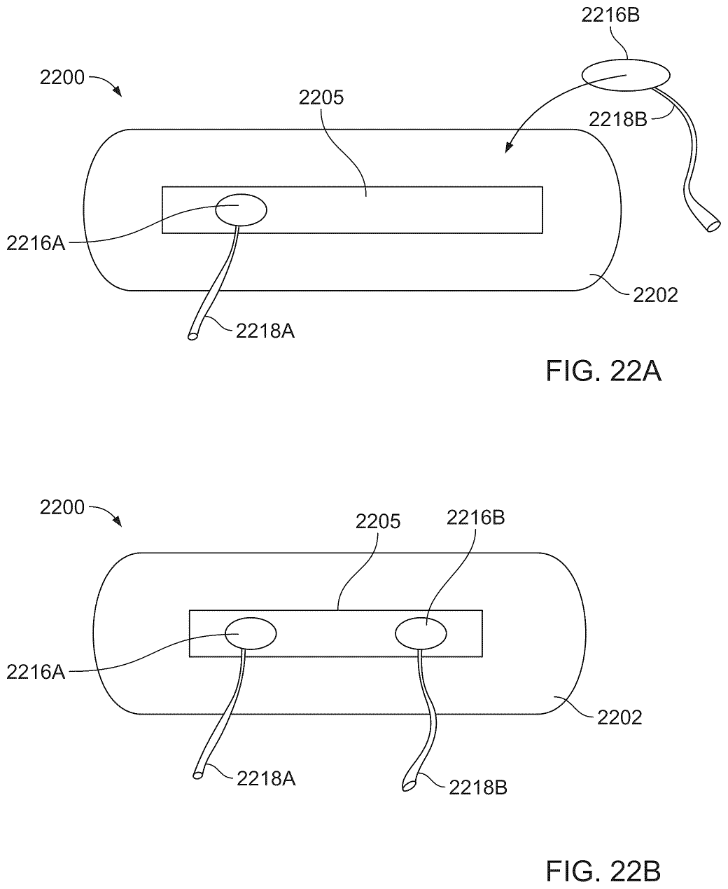

As FIG. 18C illustrates, after the dressing 1800 is cut to size and secured in place over the wound, the tubing opening 1817 can be opened or closed at resealable flap 1855 to allow or prevent air from passing through a filter into the wound contact layer 1805. The air can be directed to edges of the wound as described above. In some implementations, the tubing opening 1817 and/or the resealable flap 1855 can include, or be configured as, a filter (e.g., a HEPA filter) that can be coupled to the suction tubing 1818. The suction tubing 1818 is also connected to the central suction port 1816. In some implementations, the suction tubing 1818 can be inserted into a channel of the central suction port 1816. In some implementations, a central suction port 1816 is added to the dressing 1800 using a positionable suction hub or “lily pad” (as described below with regard to FIGS. 22A-B) or can be formed as a suction dome (as described with regard to FIGS. 1 and 3). In some implementations, the suction tubing 1818 is inserted directly into the wound contact layer 1805 without a port or dome formed over 1805 the top of the dressing or sealing layer 1802. The suction tubing 1818 can be connected to the wound contact layer 1805 with a low-profile connection to prevent or reduce the occurrence of pressure points over the wound. In some implementations, the suction tubing 1818 is connected to the wound contact layer 1805 at a variable angle or a right angle. In some implementations, suction tubing 1818 extends along a center of the dressing 1800 or is offset from a center of the dressing 1800. The suction tubing connector 1820 is coupled to a negative pressure source to provide negative pressure to the wound. As described above, in some implementations the suction tubing 1818 is connected to a collection canister or functions as a collection canister to remove exudate from the wound. In some implementations, suction tubing connector 1820 includes a controller for controlling the flow of suction from the wound. In other implementations, suction from the wound is controlled at the negative pressure source.

As depicted in FIGS. 18D and 18E, the wound contact layer 1805, sealing layer 1802 and separating tab 1821 can be customized for ease of use and to fit the wound. For example, the separating tab 1821, which can be longitudinally inward of the peelable backing 1820B of the sealing layer 1802, can be cut to size to fit a particular incision or wound on a patient. Subsequently, the sealing layer 1802 can also be cut to size to fit the particular incision or wound to be covered. In some implementations, the sealing layer 1802 and the separating tab 1821 can be separated by a distance of about 1-3 cm after being cut to size, or by a distance approximate to this range (e.g., a distance of about 0.5 cm or 4 cm). The excess portion of sealing layer 1802 extending beyond separating tab 1821 can help facilitate optimal sealing when placed against the incision or wound of the patient.

As depicted in FIG. 18F, a side view of dressing 1800 illustrates the position of separating tab 1821 relative to the tab top side 1823 and peelable backings 1820A-B of sealing layer 1802. Tab 1821 can be secured to sealing layer 1802 by tab 1823 at a first location proximate to peelable backing 1820A. Tab 1821 can extend along the length of sealing layer 1802 in a direction toward peelable backing 1820B. However, in the illustrated embodiment, tab 1821 is only secured to sealing layer 1802 at the first location proximate to peelable backing 1820A. Tab 1821 can flex downward in the direction toward peelable backing 1820 which allows it to be cut to size for ease of use and to fit a particular wound. As noted previously, sealing layer 1802 can extend in a longitudinal direction beyond tab 1821 such that tab 1821 is covered by layer 1802 before and after being cut. In other implementations, tab 1821 may be longer than sealing layer 1802 before and after being cut, or only before being cut to a size where layer 1802 is longer.

As depicted in FIG. 18G, in some implementations, separating tab 1821 can define an internal airway path starting at inflow vent 1827 and concluding at suction port 1816 and suction tubing 1818. The internal airway path can enable air to pass over the wound while traveling the longitudinal distance of tab 1821, from vent 1827 to an outermost point, and wrapping back in the opposite direction toward suction port 1816. At least one dividing wall 1825 can be used to separate the first direction of airflow along tab 1821 from the second direction of airflow toward suction port 1816. Such implementations represent an alternative to suction tubing 1818 being position along a length of tab 1821 as illustrated in the implementations of at least FIGS. 18A-E. In some such implementations, inflow vent 1827 and suction port 1816 can be offset from a midline of tab 1821 and positioned proximate to peeling backing 1820A along a length of tab 1821.

Referring still to FIGS. 18A-G, in some implementations, dressing 1800 can be prepared for use with a patient incision or wound according to the following process. First, a sealing layer 1802 and a separating tab 1821 can be provided together, with the sealing layer 1802 having a longitudinal length greater than the separating tab 1821. Both the sealing layer 1802 and the separating tab 1821 can be trimmed to appropriate lengths to accommodate a particular incision or wound to be covered. Second, tubing opening 1817 can be opened using resealable flap 1855 and adhered to a patient. Tubing opening 1817 can include a filter and can be coupled to suction tubing 1818 via the central suction port 1816. Tubing opening 1817 can allow air flow across the incision or wound that is subsequently sucked by the central suction port 1816. Tubing opening 1817 can be sealed by resealable flap 1855 to prevent air venting, or opened to allow air over the wound. Third, the separating tab 1821 can be cut to an appropriate length, followed by the sealing layer 1802 being cut to an appropriate length (e.g., appropriate for wound covering or sealing). After being cut, a distance between the separating tab 1821 and the sealing layer 1802 may be in the range of about 1-3 cm, with the sealing layer 1802 extending beyond the separating tab 1821 by this distance. The dressing 1800 can then be applied to the incision or wound of the patient according to the techniques described herein.

FIGS. 20A-J illustrate shapes of projections from an NPWT dressing to space the dressing away from a wound bed. FIG. 20A illustrates a hexagonal tiling of walls to space a dressing from a wound bed. FIG. 20B illustrates a hexagonal tiling of walls with posts in the centers of some hexagonal spaces and with walls extending to the posts. FIG. 20C illustrates a hexagonal tiling of walls with posts positioned at junctures of the walls. FIG. 20D illustrates a hexagonal tiling of walls with partial openings included with each internal wall. This allows for walls with a reduced height using a partial honeycomb hexagonal structure. FIG. 20E shows a perspective view of a hexagonal tiling of walls to space a dressing from a wound bed. At least some of the walls can include openings to permit fluid flow through the hexagonal structure. FIG. 20F illustrates an arrangement of posts connected by rods to space a dressing from a wound bed. In some implementations, the posts are randomly arranged. In other implementations, the posts are arranged in a repeating pattern. FIG. 20G illustrates a square tiling of walls with posts at the junctures of the walls. FIG. 20H illustrates an arrangement of circular walls (or alternately, circular pillars or columns). In some implementations, the arrangement can be tightly or loosely packed. FIG. 20I shows a pattern of parallelogram walls (or alternately, pillars or columns formed as parallelograms). In some implementations, the arrangement can be tightly or loosely packed. In some implementations, the walls or pillars can be formed as rectangles or squares, or any other polygonal shape. FIG. 20J illustrates a wall that is curved or wavy rather than straight. In some implementations, stripes of walls that are straight, curved, wavy, zig-zagging or having any other suitable pattern can be used to space a dressing from a wound bed.

The wound contact layer (for example wound contact layer 102) can be constructed in a multitude of different shapes or sizes as described above. For example, as described above and as illustrated in FIGS. 20A-J, the wound contact layer can include a pattern of raised walls or shapes on the wound-facing contact surface that is honeycombed, circular, triangular or rectangular/square in pattern. In some implementations, a honeycomb design of the wound-facing contact surface of the dressing is contiguous. In other implementations, a honeycomb design of the wound-facing contact surface of the dressing is non-contiguous. In some implementations, one or more edges of the honeycomb design are sharp or are rounded. In some implementations, a wound-facing contact surface of the dressing includes honeycombed walls with rounded edges on each side to reduce surface pressure at the wound surface and to prevent imprinting on the wound tissue. In some implementations, pathways or holes are formed through the raised walls or shapes to allow flow parallel to the wound surface. Alternately, in some implementations, the wound contact layer is formed as a sheet with bumps and/or posts of varying height to provide flow pathways. In some implementations, the sheet includes bumps facing the wound and bumps formed on the dorsal (opposite side of the wound) side. In some implementations, the sheet is solid. In some implementations, the sheet includes perforations. In some implementations, the design on the wound contact layer is simply ridges with undulations of different heights (for example, FIG. 20J). In some implementations, the ridges on the wound contact layer are attached to a central spine, and the ridges can have different heights and undulations to provide flow pathways along the wound edge.

A disposable dressing can be manufactured to allow for and customization of the size of the dressing to fit the wound, while still maintaining a unified design. FIG. 21 illustrates an exemplary perforated NPWT dressing 2100. The dressing 2100 can be unified in construction, meaning that the various layers including a wound contact layer 2105 and sealing layer 2102 are joined together when the dressing 2100 is removed from packaging. The wound contact layer 2100 is customizable in length or width by removal of edge 2165 along perforation 2163. The edges 2163 and 2161 are perforated to allow a medical professional to adjust the size of the wound contact layer 2105 to make it skinnier to fit a wound. The longitudinal perforations 2161 and 2163 run the length of the wound contact layer 2105. In some implementations, there can be more perforations to provide additional options in wound contact layer width or length.

In some implementations, the width of the dressing 2100 is a standard width due to the fact that the wound is already sutured close. In some implementations, the length of the dressing 2100 is adjustable by cutting one or more of the ends of the wound contact layer 2105 and adding a separate U-shaped adhesive to the dressing 2100 to create a seal or by separately cutting the wound contact layer 2105 and the sealing layer 2102 to achieve a dressing of the desired length that still is sealed air tight (see e.g., FIG. 13).

In an example, the original width of the wound contact layer 2105 is 1 inch (˜2.5 cm), and a perforation is formed 5 mm away from a center of the wound contact layer 2105 on each side to allow either 5 mm or 10 mm of polymer to be removed if the wound contact layer 2105 needs to have less width to fit the wound. This would allow for customizable length and width. In some implementations, the polymer of the wound contact layer 2105 is layered to adjust the depth or height of the dressing. In some implementations, the perforated wound contact layer 2105 includes tubing that extends through the wound contact layer 2105, for example in a branched pattern. The tubing can be uniformly patterned or varied throughout the wound contact layer 2105 and is formed so that the tubing is capable of providing irrigation and/or suction regardless of whether the wound contact layer 2105 is customized to a size by tearing or cutting at the perforations. In some implementations, the wound contact layer 2105 includes bumps or other support structures on one or both sides to separate the wound contact layer 2105 from a surface of the wound (for example, structures illustrated in FIGS. 20A-J). In addition to separating the wound contact layer 2105 from the surface of the wound, such structures provide flow pathways for irrigation across the surface of the wound.

FIGS. 22A-B illustrate an exemplary NPWT dressing 2200 with positionable suction pads 2216A-B (sometimes referred to as “lily pads”). Dressing 2200 includes sealing layer 2202, wound contact layer 2205, first positionable suction pad 2261A with suction tubing 2218A and second positionable suction pad 2261B with suction tubing 2218B. First positionable suction pad 2261A and second positionable suction pad 2261B can both be placed onto the wound contact layer 2205 in multiple positions. In some implementations, the sealing layer 2202 must be perforated before positioning the first positionable suction pad 2261A and/or second positionable suction pad 2261B on the wound contact layer 2205. In some implementations, the wound contact layer 2205 must be perforated before positioning the first positionable suction pad 2261A and/or second positionable suction pad 2261B on the wound contact layer 2205.

As described above, dressing 2200 is clear/transparent to allow direct visualization of the wound and incision through the dressing 2200 including sealing layer 2202 and wound contact layer 2205. The sealing layer 2202 uses an adhesive to attach the dressing 2200 to the patient's skin. Clear adhesive dressings can be similar to the standard drapes used currently in NPWT kits. In some implementations, the sealing layer 2202 uses a designer adhesive such as silicone or hydrocolloid. In some implementations, the sealing layer 2202 uses adhesives that allow for extended duration of adherence to the skin. In some implementations, the wound contact layer 2205 is coated with substrates to elute over time such as growth factors or antimicrobials.

### IX. Description of Kits Including Dressings

The dressings, bladders and therapeutic delivery systems described herein can be combined in a wound care kit to maximize wound healing. These kits include medications, growth factors, enzymes, mechanical debridement tools or dressings. These kits can provide a multitude of devices which in combination allow for improved healing.

In some implementations, these kits can provide sampling tools such as DNA analysis tools, bacterial assays, black lights, UV lights, antimicrobial medications, and biofilm removal therapies (medications or mechanical techniques). The kits also include instructions and algorithms for determining a best method of treatment for different types of wound, and different stages of wound healing. In some implementations, this kit can be packaged and associated with a web-based application, mobile device system, software or website. In some implementations, a research registry can be built or cross-referenced for best practices based on wound characteristics and patient demographics (e.g., age) and pre-existing diseases (e.g., diabetes).

In some implementations, tests for presence of bacterial/fungal infections are included. Florescence can be used to label certain bacterial and special lights can be used to quantify the bacterial load. Repeat measures can be used to determine efficacy of the treatment and to direct future treatments in the patient. Near infrared spectroscopy (NIRS) can be used to determine vascularity of the tissue bed. Probes for pH or other chemical characteristics can be used to determine the best means of obtaining healing.

In some implementations, the kit includes equipment for use in various setting including non-medical settings, such as wilderness settings, military settings, or sites of natural or man-made disasters. In some non-medical settings, such as a setting requiring prolonged field care, the system can include a mechanism for cleaning or sterilizing local water (e.g., tap water, ponds, rain, sea water, etc.) to purify the water for use as irrigant for a wound, or to be used to mix medications (e.g., IV fluids). For example, the system can include one or more of a UV light for use in a sterilization process to kill bacteria or other living microbes or a mechanical filter for filtering impurities from water. In some implementations, the system uses methylene blue, cold plasma, radial oxygen creation, UV light, heat, exothermic reactions, in order to sterilize the water.

### X. Use of Lower Pressures Improves Blood Flow

As described above, a dressing can be comprised of a non-collapsable/non-compressible low durometer (soft) and pliable material that allows for the use of low pressure NPWT.

In conventional systems, using sponges, low-pressure NPWT is not feasible due to compression of flow pathways through the sponge leading to high resistance when negative pressure is applied. Typically, flow pathways through a sponge may have a diameter in the range of 400-600 micron. These flow pathways are compressed when negative pressure is applied and the sponge compresses under the suction. This construct creates large/high resistance in the dressing. Other conventional absorbent dressings are designed to retain fluid and wick it away from the wound surface or retain the fluid in order to obviate the need for a suction canister or collecting canister or bag. Compression of these absorbent wicking dressings under negative suction similarly limits the use of negative pressure therapies.

The dressings described herein (see e.g., FIG. 1 and related description) are constructed in a manner to provide large-diameter flow pathways on the order of 2-3 mm in diameter. The range of diameters of the flow pathways can be from less than 0.01 mm to 5 mm or larger in order to facilitate flow. Flow is directly related to the radius of the flow pathway raised to the 4th power. Therefore, increasing the radius of a flow pathway through the dressing by 19% allows for a doubling or a 100% increase in flow through the dressing. By constructing the dressing with a hydrophobic (or hydrophilic) material such as plastic, silicone, Teflon, TPE, or other similar materials. the dressing can be constructed to enable even greater flow and less resistance. The improved flow characteristics with large diameter flow paths and hydrophobic/hydrophilic materials enable reduced negative pressure to be applied to the system and wound surface during NPWT. When lower pressures are used, mechanical (e.g., spring-loaded or other means) pumps can be used rather than conventional strong electric pumps, allowing for therapy to be applied in environments where stronger or larger pumps are not available or would be difficult to use. Conventional dressings with smaller-diameter flow pathways require strong pumps in order to overcome the large resistance withing the dressing that resists the transduction of negative pressure and the flow of exudate off the wound and towards the suction canister(s).

Compared to conventional dressings, the large-diameter flow pathway dressings described herein provide reduced bacterial growth and infection because the pathways allow for improved suction. In conventional dressings, flow through the dressing is restricted by the significant resistance within the wound contact layer and other parts of the dressing, and the dressing is designed to absorb and retain the fluid to remove it from the wound surface, instead of allowing fluid to be removed with suction. However, when conventional dressings including sponges become saturated, fluid is retained on the wound surface. Clinically, if suction is lost for as short as 2 hours, recommendations are to remove conventional dressings completely to avoid wound and skin breakdown and infection, because a sponge retaining fluid at the wound surface encourages bacterial overgrowth and biofilm creation. Bacterial colonization and biofilm creation inhibit wound healing and can result in impaired healing and even systemic infections/complications. The dressings described herein are non-absorptive so that the wound remains clear of bacterial overgrowth and fluid is removed through the dressing by application of suction. Reduced bacterial growth additionally reduces odor, redness, puss, and swelling of the wound surface and surrounding tissue observed at dressing changes. Because of the high resistance and absorptive nature of conventional clinical dressings, such dressings are unable to tolerate a negative pressure value of less than 100 mmHg.

Compared to conventional dressings, the large-diameter flow pathway dressings described herein reduce the incidence of positive pressures on the wound surface and are able to provide therapeutic negative pressure to a wound at much lower values compared to conventional dressings (e.g., −20 to −95 mmHg). When negative pressure is applied to traditional foam dressings, the sponge or foam of the dressing collapses on itself. A negative pressure applied to the system results in a positive compressive pressure or surface contact pressure on the wound surface, such that the use of compressive materials in a dressing increases the contact surface pressure on the wound. In experiments, −125 mmHg pressure placed on a traditional foam NPWT dressing resulted in over 200 g/cm² contact pressure at the wound surface. Additionally, depending on the size of the dressing and other characteristics, the positive pressure applied to a wound surface can exceed 150 mmHg. When such positive pressures are applied to the wound surface, the wound surface becomes ischemic due to restricted blood flow and healing is disrupted or prevented. Additionally, without blood flow, nutrients are not brought to the wound which further slows or disrupts the healing process. Conversely, at −50 mmHg negative pressure there is approximately 30 g/cm² of positive pressure or 20 mmHg. At this pressure, perfusion is not only not reduced, but it is actually increased.

Low positive pressure incident on the wound surface and periwound has been shown to promote blood flow and perfusion. Within the range of +10 to +30 mmHg of compression applied to a wound surface, increased blood flow is observed. For example, this phenomenon is seen in compressive sleeves and leggings worn by athletes, and compression stockings also provide compression in this range in order to promote blood flow and improve healing. By improving dressing design to create a non-compressible dressing with improved flow allowing for use of lower negative pressure settings, the dressing described herein (see e.g., FIG. 1) results in increased blood flow under the dressing instead of creating an ischemic wound bed with low positive pressure application as is seen in traditional NPWT dressings.

A non-compressible or non-collapsible dressing, as described herein, reduces these positive pressures applied at the wound surface. In experiments, when −125 mmHg of suction were applied to non-compressible dressings as described above, the contact pressure felt at the wound surface was roughly 100 g/cm² or less, and positive pressure at the wound surface was about 60 mmHg or less. Accordingly, the positive pressure and contact pressure applied at the wound surface by non-compressible dressings as described herein are significantly lowered and improved compared to pressures resulting from use of a traditional NPWT dressing under the same applied suction. Additionally, because the design of the dressing incorporates both non-compressible material with expanded large-diameter flow pathways compared to conventional dressings, a much lower negative pressure can be utilized to achieve similar therapeutic effects.

Additionally, intermittent suction has been shown to improve wound healing when used with traditional NPWT dressings. This fact is likely due to the tissue being able to re-perfuse during low-pressure application of the intermittent/cyclic suction since the positive pressure applied to the wound surface by conventional dressings causes ischemia.

However, intermittent suction is not currently widely used for several reasons. First the compressed sponge allows for tissue ingrowth. When the pressure is reduced, the sponge expands as there is some inherent recoil with reduced suction. This sponge expansion at the wound surface results in pulling of the tissue that has grown into the sponge from the wound bed resulting in pain. Second, intermittent or cyclic suction is not ideal in sponge dressings due to the recoil or expansion of the sponge with reduced pressure. The expansion of the sponge pushes up and against the sealing drape or adhesive layer used to create a seal for the system to apply negative pressure. In many cases, the wound will have exudate and fluid within the wound be or periwound. The fluid contributes to the loss of a seal or creation of a leak. With reduced suction, water is releases since the dressing is designed to absorb and retain fluid thereby degrading the seal. Third, the reduced suction allows the sponge to expand against the sealing layer. This expansion pushes against the sealing layer creating leaks. When a sponge is placed, the sealing layer is placed over the sponge. Obtaining a good seal can be difficult initially due to the sponge compressing and then pushing against the seal. Once negative pressure is applied, the compression of the sponge pulls the sealing layer down to the wound and periwound which reinforces the seal. When that suction is removed completely or partially, the compressive forces on the sealing layer are also removed and the sponge pushes against the sealing layer resulting in a lifting off of the sealing layer and leak creation.

However, a dressing such as that described herein, which is designed to be non-compressible/non-collapsible, overcomes these concerns related to the use of intermittent or cyclic application of pressure. When pressures are changed (increased or decreased) the dressing will not exert a force on the sealing layer. Additionally, if the dressing is designed to not retain fluid or absorb fluid, then there is no concern for reduced suction or negative pressure as there will be no leaking of fluid. If the dressing is designed not to allow for tissue ingrowth, then there will be no pain with reduced suction and no tearing of tissue away from the wound surface with expansion because there is no expansion and no attachment to the tissue.

In some implementations, a dressing system is designed for the specific use of variable suction to create a high positive pressure in the wound to pump out edema or fluid in a similar manner to sequential compression devices placed on the lower leg to prevent blood clots (DVT's). Higher pressures followed by gradual reduction in pressure in a cyclic manner may provide improved flow and healing. Since the non-compressive and non-absorptive dressing avoids tissue ingrowth and compression and thereby expansion, variable suction may provide improved healing. With traditional gauze or sponge dressings, variable and low pressure settings have not been achievable.

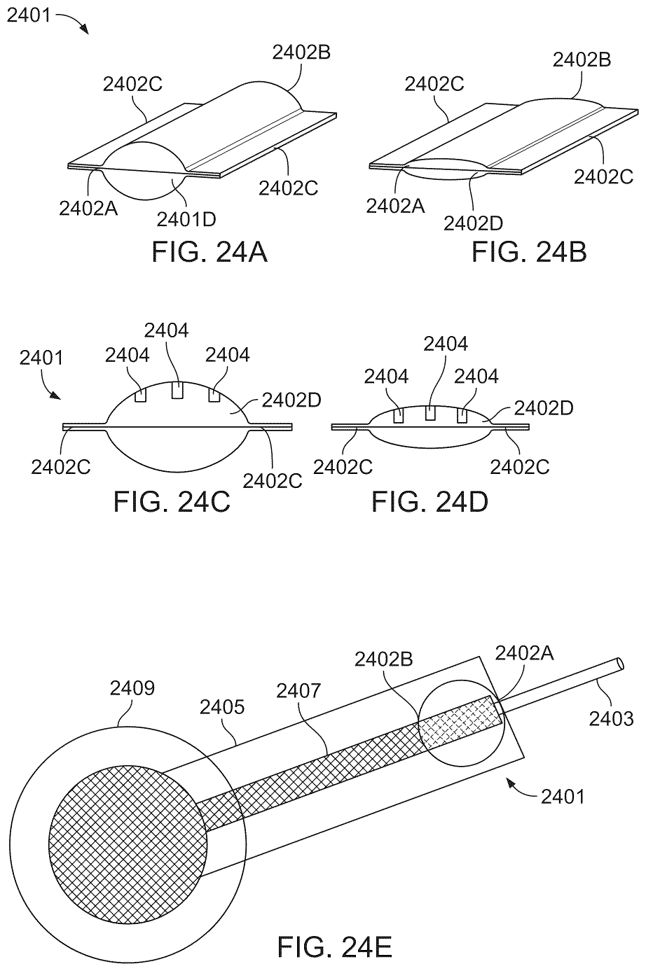

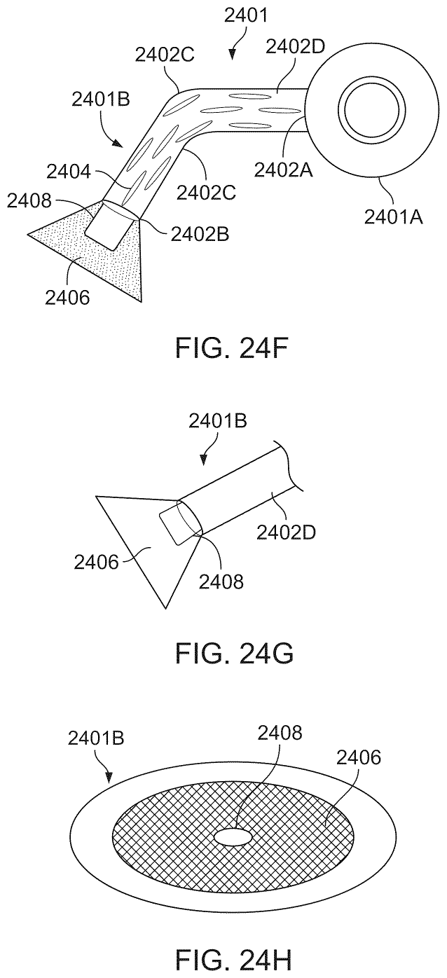

Referring now to FIGS. 24A-H, in some implementations, a suction dome 2401 can have a flat collapsible configuration with a first end 2402A separated from a second end 2402B along opposing edges 2402C. Suction dome 2401 can include a lumen 2402D defined between the first and second ends 2402A and 2402B. The lumen 2402D can be collapsed or flattened when a force is applied against an exterior surface of the suction dome 2401. In some implementations, suction dome 2401 can include one or more elements 2404 configured as bumps (e.g., gel bumps), ridges, channels, or other uneven surfaces to prevent the lumen 2402D from collapsing under negative pressure. In some implementations, the one or more elements 2404 can be made from soft materials to avoid pressure concentrations within the lumen 2402D.

FIG. 24C illustrates some implementations where the suction dome 2401 is not subjected to negative pressure and the lumen 2402D is open for fluid flow. Conversely, FIG. 24D illustrates some implementations where the suction dome 2401 is subjected to negative pressure which forces the lumen 2402D to compress. The one or more elements 2404 prevent the lumen 2402D from completely flattening so that consistent fluid flow through the lumen 2402D can be maintained. FIG. 24E illustrates some implementations of the suction dome 2401 with tubing 2403 coupled to the first end 2402A, with sealing layer 2405, wound covering or filler 2407, and an adhesive drape 2409 coupled to the second end 2402B. Fluid can be transferred from the wound location through lumen 2402D and out through the tubing coupled to the first end 2402A.

In some implementations, suction dome 2401 can include a first portion having a flat collapsible configuration as described above, and a second portion having a configuration similar to suction dome 301 or another suction dome described herein. For example, as illustrated in FIGS. 24F-H, suction dome 2401 can include a first portion 2401A and a second portion 2401B having an internal lumen 2402D defined between first and second ends 2402A and 2402B between opposing edges 2402C. The second portion 2401B can include one or more elements 2404 to prevent the lumen 2402D from collapsing when subjected to negative pressure and to maintain consistent flow through the lumen 2402D. The second portion 2401B can include an adhesive layer 2406 configured to stick on a patient's skin when a non-adhesive peeling material is removed from the adhesive layer 2406. The second portion 2401B can also include at least one aperture 2408 in the adhesive layer 2406. In some implementations, the aperture 2408 can include exposed adhesive that can be adhered to a portion of the first portion 2401A of the suction dome 2401. Removing the non-adhesive peeling material from the remainder of the adhesive layer 2406 (e.g., everything but aperture 2408) can enable adhesion of the entire adhesive layer 2406 to a wound.

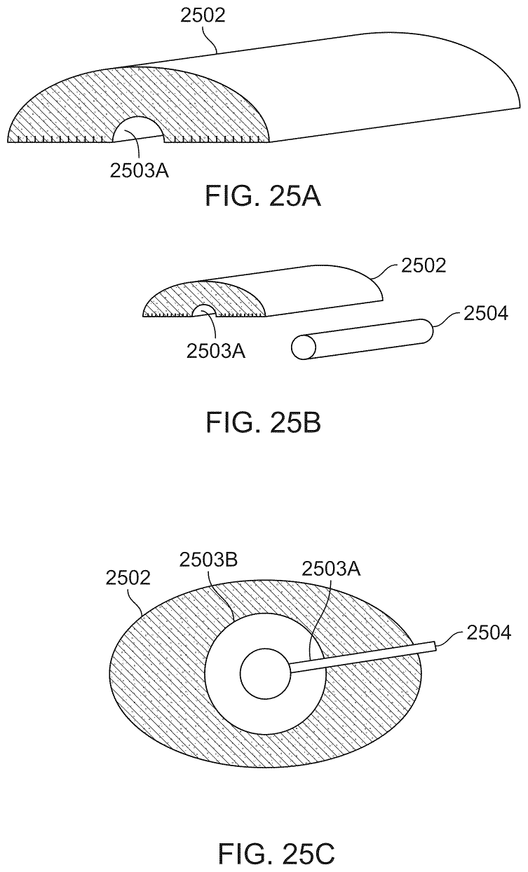

Referring now to FIGS. 25A-C, in some implementations, an external padding 2502 can be positioned over tubing 2504 and/or a dressing to prevent potential pressure buildup which could cause injuries to patients. The external padding 2502 can define a first cutout 2503A configured to receive tubing 2504 as illustrated in FIGS. 25B and 25C. The external padding 2502 can define a second cutout 2503B configured to receive a dressing which itself is coupled to the tubing 2504. A bottom surface 2503C of the external padding 2502 can include an adhesive or padding layer for coupling the external padding 2502 to the dressing and/or a patient.

In some implementations, the external padding 2502 can be positioned around the tubing 2504, for example above and/or below the tubing 2504. In some implementations, the external padding can be made out of foam or gel (non-compressive), or another similar material. In some implementations, the external padding 2502 can be positioned outside the area placed under negative pressure. In some implementations, the external padding 2502 can have a torus geometry or a cylindrical geometry such as a hemispherical cylinder. The first cutout 2503A can be defined through a midpoint of the external padding 2502 in some implementations, or defined through a point offset from the midpoint in other implementations.

Some implementations can enable evacuation of fluid from an incision or wound using lower or reduced negative pressures when compared to certain conventional wound dressing systems or devices. The lower negative pressures may be used continuously or intermittently in some implementations, and optionally with optimized flow paths such as those illustrated in FIGS. 2 and 3C-3G. By operating at lower pressures (e.g., −20 mmHg to −75 mmhg), some implementations can produce less positive pressure against the wound surface which can result in improved patient outcomes (e.g., less positive pressure yields less blood flow reduction to an incision or wound bed, thereby resulting in faster healing for the patient). For example, when a vacuum applies negative pressure to a wound dressing, a reciprocal positive pressure is applied by the dressing against the underlying tissue that the dressing covers. If the wound dressing is too resistant to flow, which is the case for some porous foam sponges, then more negative pressure is required to achieve the same rate of fluid evacuation as in circumstances where the wound dressing is less resistant to flow. This can result in a reciprocal consequence of more positive pressure applied against the wound surface which results in more blood flow reduction and slower healing for the patient.

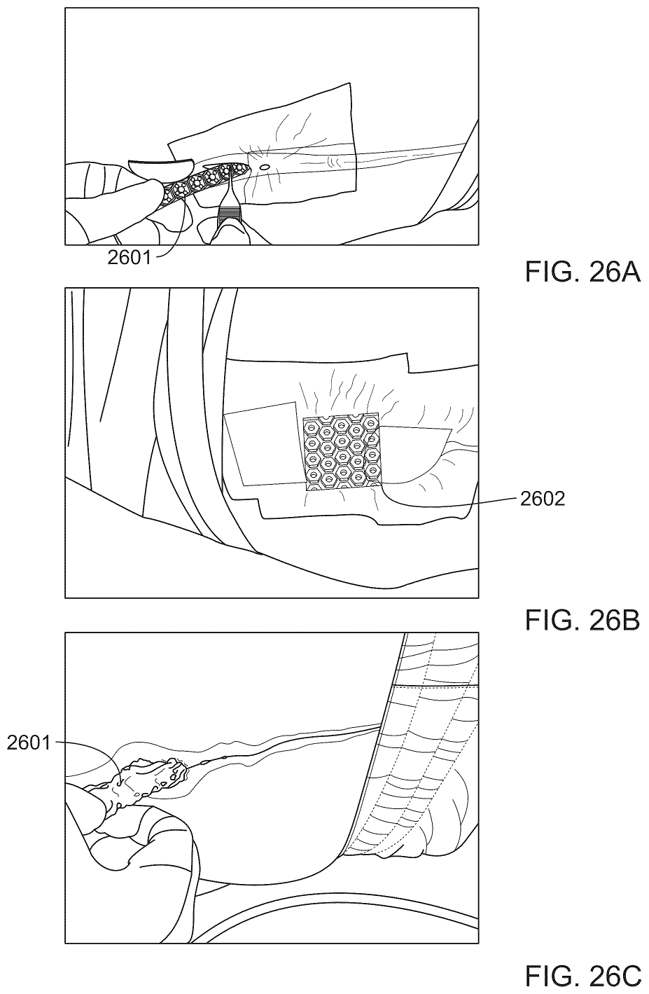

Referring now to FIGS. 26A-C, a dressing 2601 can be inserted into a pathway of a tunnel wound and slid down the pathway to enable negative pressure application throughout the wound. For example, FIG. 26A illustrates the dressing 2601 being inserted into the tunnel wound pathway, FIG. 26B illustrates the dressing 2601 positioned within the tunnel wound pathway and configured to receive a suction dome, and FIG. 26C illustrates the dressing 2601 being removed from the tunnel wound pathway. In some implementations, the dressing 2601 can comprise a double-sided perforated sheet. An external side of the perforated sheet that faces the wound can include bumps or can be smooth. An end of the dressing 2601 that extends through the tunnel wound pathway can include a reinforced area that is configured to receive a placement rod along a longitudinal midsection of the dressing 2601. The placement rod, which can be a metal or polymer stiff rod in some implementations, can be used to force the dressing 2601 through the tunnel wound pathway for placement therein. Once positioned at a distal end of the tunnel wound pathway (e.g., distal from the insertion end), the placement rod can be removed and excess dressing 2601 extending out of a proximal end can be removed. Subsequently, a sealing drape or layer 2602 and a suction dome can be used with the dressing 2601. The sealing drape or layer 2602 can be replaced over time, with the dressing 2601 being removed from the tunnel wound a short distance at each replacement interval. Once the dressing 2601 is backed out from the tunnel wound and cut to size, a new sealing drape or layer 2602 can be placed against the wound and the suction dome can be reapplied. In some implementations, a suction dome can be configured so that it fits into a central pathway of the dressing 2601 where the placement rod is used. For example, the suction dome can be configured so that it fits within the dressing 2601 to ensure optimal suction from the tunnel wound.

In some implementations, the dressing 2601 can extend along the tunnel wound pathway such that it extends partially outward from the proximal and distal ends of the tunnel wound (e.g., through an opening on both ends of the tunnel wound). Accordingly, the dressing 2601 can be configured to provide irrigation to the tunnel wound along the entire pathway and at the proximal and distal ends. A suction dome can be placed at each end to provide irrigation to one end and suction to the other end (e.g., irrigation to the proximal end and suction to the distal end). In some implementations, this configuration of the dressing 2601 and the suction domes can be used to irrigate infected hardware or implants of the patient. This configuration can also enable lavage of wounds and implants while the patient is located in a non-operating room setting (e.g., when the patient is at home). This is not possible with certain conventional wound dressings, such as sponges, because they unavoidably collect bacteria (or other wound contaminants) and allow for bacteria colonization which can be harmful to the wound.

Embodiments of some implementations described herein can include a drain tube connected using either a Y-shaped connector with irrigation tubing, or connected to a suction dome. Single or multiple irrigation tubing appendages (e.g., similar to a Jackson-Pratt drain or a Penrose drain) can enable irrigation of deep sections of a wound. Including a drain tube with single or multiple irrigation can also allow areas with implants or tunneling wounds to be irrigated preferentially over other areas. In some implementations, single or multiple irrigation tubing appendages can also allow medication to be delivered independent from the rest of the wound. This could be beneficial for biofilm busters, chemotherapy for cancers, or for specific antimicrobials designed for cleaning implants. Independent tubing can also enable targeted or directed placement of irrigants or medication within the wound, with the irrigants or medication being subsequently sucked out with a suction dome across the remainder of the wound. In some implementations, treatment regimens can be used where medication is first provided to the wound and then, after a dwell time, a neutralizing agent is provided to neutralize the reaction caused by the medication.

Embodiments of some implementations described herein can be used with a suction pump configured to operate in reduced negative pressure ranges (e.g., a pressure range of about −20 mmHg to −75 mmhg). At these reduced pressure ranges, exudate can still be removed from the wound unlike with certain conventional systems such as foam or sponge dressings, or absorbent dressings. In some implementations, the suction pump can be powered using one or more batteries (optionally rechargeable), solar energy, mechanical energy, and/or body heat, for example. In such implementations and others, the power needed to provide suction at any level of negative pressure can be significantly reduced due to a lack of resistance associated with a wound dressing configured to be non-compressible under negative pressure. In some implementations, the suction pump can be disabled after a period of time (e.g., hours or days, 30 days). In some implementations, a security code can be used with the suction pump to resume operation when disabled after operating for the period of time. In some implementations, the suction pump can be at least partially disposable which reduces or eliminates servicing needs. In some implementations, the suction pump can be tracked via GPS and can be configured for bilateral (incoming and outgoing) communication with an external electronic device.

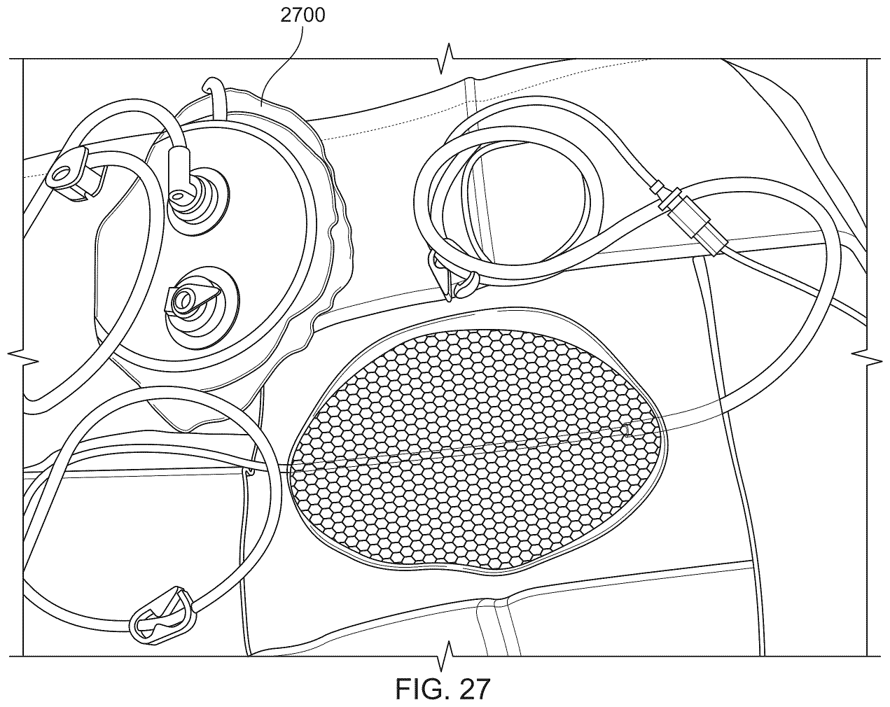

In some implementations, the suction pump can be compatible with a suction cannister having an increased capacity (e.g., 1L or more) to accommodate high volume wound irrigation. Referring now to FIG. 27, a suction cannister 2700 may be in the shape of a foldable bag or another compressible form, and can be refilled and reused after emptied. This can be beneficial compared to certain conventional suction cannisters which can be expensive to make, require large space to store, and are generally non-reusable. In some implementations, suction can be provided using a spring-loaded suction pump (e.g., Hemovac® from Zimmer Biomet). Suction can be provided using the spring-loaded pump with negative pressure or as irrigation provided to the wound. Example settings where spring-loaded pump suction can be used include a patient's home, field operations for military service members, or in underdeveloped areas of the world.

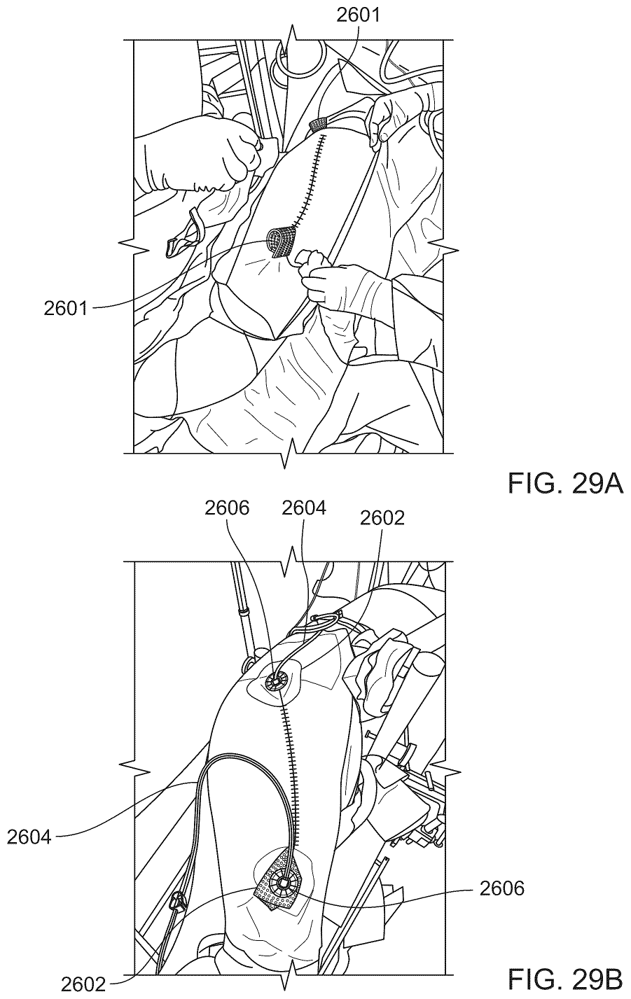

Referring now to FIGS. 29A-B, a dressing 2601 can be inserted through a wound with a first exposed dressing portion on one end of the wound and a second exposed dressing portion on the other end of the wound. The dressing 2601 can be folded prior to insertion into the wound. A sealing drape or layer 2602 with a suction dome 2604 can be placed over each exposed portion of the dressing 2601. Suction can be placed on one end of the wound, via a first sealing drape or layer 2602 and a first suction dome 2604, while inflow can be placed on the other end of the wound, via a second sealing drape or layer 2602 and a second suction dome 2604. This can provide irrigation to the wound without the patient returning to the operating room.

A number of embodiments of the invention have been described. Nevertheless, it will be understood that various modifications may be made without departing from the scope of the invention. Accordingly, other embodiments are within the scope of the following claims.

### EMBODIMENTS

### A. Transparent Dressing A

1. A NPWT assembly comprising:
   a dressing comprising:
      a wound contact layer comprising a transparent material; and
      a sealing layer comprising an adhesive.
2. The NPWT assembly of claim 1, wherein the dressing is directional.
3. The NPWT assembly of claim 1 or 2, wherein a wound-facing surface of the wound contact layer is configured to be smooth.
4. The NPWT assembly of any of claims 1-3, wherein a wound-facing surface of the wound contact layer is configured to be rough.
5. The NPWT assembly of any of claims 1-4, wherein a wound-facing surface of the wound contact layer is configured to be perforated.
6. The NPWT assembly of any of claims 1-5, wherein a wound-facing surface of the wound contact layer is configured to be impermeable.
7. The NPWT assembly of claim 1, wherein the dressing is non-directional.
8. The NPWT assembly of claim 7, wherein the wound contact layer is symmetric.
9. The NPWT assembly of any of claims 1-8, wherein the wound contact layer comprises a passageway structure.
10. The NPWT assembly of claim 9, wherein one or more passageways of the passageway structure have a diameter of between 0.1 mm and 5 mm.
11. The NPWT assembly of any of claims 1-10, wherein the wound contact layer comprises a means to change a thickness of at least a portion of the wound contact layer from a first thickness to a second thickness.
12. The NPWT assembly of claim 11, wherein the means to change a thickness of at least a portion of the wound contact layer comprises an expandable bladder.
13. The NPWT assembly of claim 11, wherein the means to change a thickness of at least a portion of the wound contact layer comprises a material configured to expand upon exposure to a stimuli.
14. The NPWT assembly of claim 11, wherein the depth of the wound contact layer is about 1-6 mm.
15. The NPWT assembly of claim 11, wherein the depth of the wound contact layer is about 2-50 mm.
16. The NPWT assembly of any of claims 1-15, wherein the transparent material comprises a polymer.
17. The NPWT assembly of claim 16, wherein the polymer is a thermoplastic elastomer.
18. The NPWT assembly of any of claims 1-17, wherein the transparent material comprises silicone.
19. The NPWT assembly of any of claims 1-18, wherein the transparent material is configured to prevent tissue ingrowth.
20. The NPWT assembly of any of claims 1-19, wherein the transparent material is non-absorbent.
21. The NPWT assembly of any of claims 1-20, wherein the transparent material is non-compressible.
22. The NPWT assembly of any of claims 1-21, wherein the transparent material is non-porous.
23. The NPWT assembly of any of claims 1-22, wherein the transparent material is moldable.
24. The NPWT assembly of any of claims 1-23, wherein the transparent material is configured to mold to a shape of a wound bed.
25. The NPWT assembly of any of claims 1-24, wherein the wound contact layer is formed by 3D-printing.
26. The NPWT assembly of any of claims 1-24, wherein the wound contact layer is formed by injection molding.
27. The NPWT assembly of any of claims 1-26, further comprising a negative pressure pump, the negative pressure pump configured to be coupled to through the sealing layer and to provide a negative pressure to the wound contact layer.
28. The NPWT assembly of claim 27, wherein the negative pressure pump in fluid communication with one or more passageways of the passageway structure.
29. The NPWT assembly of any of claims 1-28, wherein the wound contact layer comprises one or more therapeutic substances.
30. The NPWT assembly of claim 29, wherein the one or more therapeutic substances comprise at least one of medicine, ions, chemicals, biologics, synthetic proteins, synthetic materials.
31. The NPWT assembly of claim 29, wherein the one or more therapeutic substances comprise an anti-bacterial substance.
32. The NPWT assembly of claim 29, wherein the one or more therapeutic substances comprise a chemical to promote vascularization, epithelialization, or granulation.
33. The NPWT assembly of claim 29, wherein the one or more therapeutic substances are positioned in layers on the wound contact layer.
34. The NPWT assembly of claim 29, wherein the layers of the one or more therapeutic substances on the wound contact layer are configured for controlled release over a period of time.
35. The NPWT assembly of any of claims 1-34, wherein the wound contact layer is configured to elute particles, ions, chemicals, proteins, or other elements upon exposure to bodily fluids, enzymes or reagents.
36. The NPWT assembly of any of claims 1-35, wherein the NPWT assembly is configured to be positioned over a wound for more than seven days.
37. The NPWT assembly of any of claims 1-36, wherein the sealing layer is transparent.
38. The NPWT assembly of any of claims 1-37, wherein the wound contact layer and sealing layer are configured to allow UV light to be transmitted through to the wound bed.
39. The NPWT assembly of claim 38, wherein the wound contact layer comprises a layer of a substance configured to be activated by the UV light.
40. The NPWT assembly of claim 39, wherein the substance is a medication or an anti-bacterial substance.
41. The NPWT assembly of claim 39, wherein the substance is chemotherapeutic.
42. The NPWT assembly of any of claims 1-41, wherein the NPWT assembly further comprises a UV-light source positioned in the sealing layer, the UV-light source configured to provide light through the wound contact layer to the wound bed.
43. The NPWT assembly of claim 42, wherein the wound contact layer is configured to reflect the light to amplify the light across the wound bed.
44. The NPWT assembly of any of claims 1-43, wherein the wound contact layer comprises wiring extending through the wound contact layer.
45. The NPWT assembly of claim 44, wherein the wound contact layer is configured to be cut to a size of the wound bed, and wherein the wiring is configured to be operable after cutting.
46. The NPWT assembly of claim 44, wherein the wiring is configured in parallel throughout the wound contact layer.
47. The NPWT assembly of claim 44, wherein the wiring is configured to provide heat to the wound bed.
48. The NPWT assembly of claim 44, wherein the wiring is configured to provide an electromagnetic pulse to the wound bed.
49. The NPWT assembly of claim 44, wherein the wiring is configured to provide visible light to the wound bed.
50. The NPWT assembly of claim 44, wherein the wiring is configured to be connected to a sensor at the wound bed.
51. The NPWT assembly of claim 50, wherein the sensor comprises a pressure sensor or a temperature sensor.
52. The NPWT assembly of claim 44, wherein the wiring is configured to provide Near Infrared Spectroscopy (NIRS).
53. The NPWT assembly of any of claims 1-52, wherein the wound contact layer is configured to provide a therapeutic substance to the wound bed.
54. The NPWT assembly of claim 53, wherein the therapeutic substance is a fluid, a gel, a gas or a solid.
55. The NPWT assembly of claim 53, wherein the therapeutic substance is a bactericide.
56. The NPWT assembly of claim 53, wherein the bactericide is one of betadine, chlorhexidine, hydrogen peroxide, Dakins solutions.
57. The NPWT assembly of claim 53, wherein the NPWT assembly is configured for use in a cancerous lesion or resection bed and the wound contact layer is configured to provide a chemotherapeutic agent to the wound bed.
58. The NPWT assembly of claim 53, wherein the wound contact layer is configured to provide the therapeutic substance to the wound bed through at least one passageway.
59. The NPWT assembly of claim 1-58, wherein the wound contact layer includes one or more passageways configured to provide irrigation to the wound bed.
60. The NPWT assembly of claim 59, wherein the at least one of the one or more passageways configured to provide irrigation comprise a plurality of irrigation pathways.
61. The NPWT assembly of claim 60, wherein the plurality of irrigation pathways are arranged radially from a center of the wound contact layer.
62. The NPWT assembly of claim 60, wherein the plurality of irrigation pathways are arranged in a branching pattern.
63. The NPWT assembly of claim 59, wherein at least one of the one or more passageways of the wound contact layer are configured to provide a gas to the wound bed.
64. The NPWT assembly of claim 59, wherein at least one of the one or more passageways of the wound contact layer are configured to provide suction to the wound bed.
65. The NPWT assembly of any of claims 1-64, further comprising a means for applying mechanical motion to the wound bed.
66. The NPWT assembly of claim 65, wherein the means for applying mechanical motion comprise a pneumatic inflow, a bladder, or a vibration mechanism.
67. The NPWT assembly of any of claims 1-66, further comprising a docking station arranged in a non-wound-contacting face of the wound contact layer, and a suction dome configured to lock into the docking station.
68. The NPWT assembly of claim 67, wherein the sealing layer is configured to be positioned around the suction dome.
69. The NPWT assembly of claim 67, wherein the suction dome is configured to provide irrigation and/or suction to the one or more passages of the wound contact layer.
70. The NPWT assembly of claim 63, wherein a first passageway through the suction dome is configured to provide inflow to the wound bed and a second passageway through the suction dome is configured to provide outflow from the wound bed.
71. The NPWT assembly of any of claims 1-70, wherein the wound contact layer is configured to be non-collapsible and non-compressible.
72. The NPWT assembly of any of claims 1-71, wherein the wound contact layer is configured to be non-absorptive.
73. The NPWT assembly of any of claims 1-72, wherein the wound contact layer is configured to maintain a positive surface pressure at a wound bed under −30 to −90 mmHg pressure.
74. The NPWT assembly of any of claims 1-72, wherein the wound contact layer is configured to maintain a positive surface pressure at a wound bed under −20 to −75 mmHg pressure.

### B. Transparent Dressing B

1. A wound dressing comprising:
   a transparent polymeric wound contact layer comprising:
      a wound-facing surface;
      a top surface opposite the wound-facing surface; and
      a docking port formed in the top surface, the docking port comprising a central suction hub, wherein the central suction hub is coupled to a plurality of passageways extending through the wound contact layer between the wound-facing surface and the top surface.
2. The wound dressing of claim 1, wherein the docking port is configured to mate with a suction dome, the suction dome engaging the central suction hub to provide an air-tight seal.
3. The wound dressing of claim 1 or 2, wherein the docking port further comprises an external wall surrounding and spaced apart from the central suction hub.
4. The wound dressing of claim 3, wherein the external wall and the central suction hub define an irrigation well in the docking port.
5. The wound dressing of claim 4, wherein the irrigation well is fluidically coupled to at least some of the plurality of passageways.
6. The wound dressing of claim 4 or 5, wherein the suction dome is configured to provide suction and irrigation to the wound through the plurality of passageways.
7. The wound dressing of any of claims 1-6, wherein the wound contact layer comprises an adhesive skirt coupled to the top surface, the adhesive skirt configured to engage a patient's skin surrounding a wound.
8. The wound dressing of any of claims 1-6, further comprising a sealing layer configured to be positioned over the top surface of the wound contact layer.
9. The wound dressing of any of claims 1-8, wherein the wound contact layer is configured to be non-collapsible.

### C. Transparent Dressing C

1. A wound dressing comprising:
   a transparent polymeric wound contact layer, and
   at least one coating formed on a wound-facing surface of the transparent polymeric wound contact layer, the at least one coating comprising a therapeutic substance.
2. The wound dressing of claim 1, wherein the at least one coating is configured to be released over a predetermined period of time based on a thickness of the at least one coating.
3. The wound dressing of claim 1 or 2, wherein the at least one coating is configured to be released over a predetermined period of time based on an interaction of the at least one coating with a substance produced by an open wound.
4. The wound dressing of any of claims 1-3, wherein the at least one coating is configured to be released over a predetermined period of time based on an interaction of the at least one coating with a substance supplied to the wound.

### D. Dressing Method

1. A method of treating a wound, the method comprising:
   positioning a wound contact layer in a wound;
   engaging a docking port of the wound contact layer with a suction dome to connect the wound contact layer to a suction source and irrigation source; and
   providing at least one of suction and irrigation to the wound through the suction dome.
2. The method of claim 1, further comprising:
   securing the wound contact layer in the wound.
3. The method of claim 2, wherein securing the wound contact layer in the wound further comprises:
   positioning a sealing layer over the wound contact layer, the sealing layer comprising adhesive edges configured to attach to a patient's skin surrounding the wound and to provide an airtight seal over the wound contact layer.
4. The method of any of claims 1-3, further comprising:
   cutting an aperture through the sealing layer to expose the docking port prior to engaging the docking port with the suction dome.
5. The method of any of claims 1-4, further comprising:
   cutting the wound contact layer to a size of the wound before positioning the wound contact layer in the wound.
6. The method of any of claims 1-5, further comprising:
   positioning more than one wound contact layer in the wound to fit a depth of the wound.
7. The method of any of claim 1-6, further comprising:
   providing at least one therapeutic agent to the wound via the wound contact layer.
8. The method of claim 7, wherein the providing at least one therapeutic agent to the wound comprises providing at least one coating comprising a therapeutic agent to a wound-facing surface of the wound contact layer.
9. The method of claim 7, wherein the providing at least one therapeutic agent to the wound comprises providing the at least one therapeutic agent from the irrigation source.
10. The method of any of claims 1-7, wherein the providing at least one of suction and irrigation to the wound through the suction dome further comprises providing a pressure between −30 and −90 mmHg to the wound.
11. The method of any of claims 1-7, wherein the providing at least one of suction and irrigation to the wound through the suction dome further comprises providing a pressure between −20 and −75 mmHg to the wound.

### E. EVRA

1. An electronic vacuum regulator system comprising:
   a base comprising:
      a housing;
      an inflow port;
      an outflow port; and
      a pump apparatus positioned within the housing, the pump apparatus configured to provide suction through the inflow port; and
   a control system coupled to the base, wherein the control system is detachable from the base, the control system configured to:
      control suction provided by the pump apparatus through the inflow port.
2. The system of claim 1, wherein the base further comprises a cavity and a docking port within the cavity, the docking port in fluid communication with the outflow tubing.
3. The system of claim 2, wherein the base further comprises a reaction chamber positioned between the docking port and the outflow tubing.
4. The system of claim 2, wherein the cavity is sized and configured to accept a therapeutic cartridge.
5. The system of claim 4, wherein the docking port within the cavity is sized and configured to engage with the therapeutic cartridge.
6. The system of claim 5, wherein the docking port is configured to puncture a seal of the therapeutic cartridge when the docking port is engaged with the therapeutic cartridge.
7. The system of claim 5 or 6, wherein the cavity further comprises a scanning device configured to scan a label of the therapeutic cartridge when the therapeutic cartridge is engaged and transmit information from the scanned label to the control system.
8. The system of claim 7, wherein the label comprises one of a barcode, a QR code, or an RFID tag.
9. The system of claim 7 or 8, the control system further configured to:
   identify a therapeutic cartridge based on information received from the scanning device; and
   provide instructions to deliver a compound extracted from the therapeutic cartridge through the outflow port.
10. The system of any of claims 1-9, the control system further configured to:
   provide instructions to provide irrigation through the outflow port.
11. The system of any of claims 1-10, the control system further configured to:
   transmit a location of the control system to an external device.
12. The system of any of claims 1-11, the control system further configured to:
   display a message to a user.
13. The system of any of claims 1-11, the control system further configured to:
   transmit information related to details of use of the base to an external device.

### F. EVR B

1. An electronic vacuum regulator (EVR) system comprising:
   a housing; and
   a receptacle formed in the housing, the receptacle sized and configured to receive a canister of medicament for distribution to a wound dressing through the housing.
2. The EVR system of claim 1, further comprising:
   means for detection of identification of a medicament within a canister when the canister is positioned in the receptacle.
3. The EVR system of claim 2, further comprising:
   a controller configured to distribute the medicament to a wound dressing through the housing based on stored instructions associated with the detected identification of the medicament.
4. The EVR system of claim 2 or 3, further comprising:
   a controller configured to store a record associated with the detected identification of the medicament.
5. The EVR system of any of claims 1-4, further comprising:
   an irrigation outlet coupled to irrigation tubing, the EVR configured to distribute the medicament to the wound through the irrigation outlet and irrigation tubing.

### G. Dressing (Leaflet)

1. A NPWT dressing assembly for use in multi-cavity wound, the NPWT dressing assembly comprising:
   a plurality of dressings comprising at least a first dressing and a second dressing, each of the plurality of dressings comprising:
      a wound contact layer comprising a polymer material and an irrigation pathway extending throughout the wound contact layer;
   wherein the first dressing of the plurality of dressings is coupled to the second dressing by an irrigation tube connecting the irrigation pathway of the first dressing to the irrigation pathway of the second dressing.
2. The NPWT dressing assembly of claim 1, wherein the polymer material is transparent.
3. The NPWT dressing assembly of claim 1, wherein the polymer material is translucent.
4. The NPWT dressing assembly of any of claims 1-3, wherein the irrigation tube is brightly colored.
5. The NPWT dressing assembly of any of claims 1-4, wherein the plurality of dressings is configured to be cut to fit the multi-cavity wound, and wherein the irrigation pathway is configured to allow fluid to flow through the irrigation pathway to the plurality of dressings even after a portion of the irrigation pathway is cut.
6. The NPWT dressing assembly of any of claims 1-5, further comprising an elongate flag extending from at least one of the plurality of dressings, the elongate flag configured to be positioned external to the multi-cavity wound to allow a user to pull the at least one of the plurality of dressings from the multi-cavity wound by pulling on the elongate flag.
7. The NPWT dressing assembly of any of claims 1-6, further comprising:
an electronic vacuum regulator (EVR) system having an inlet and an outlet;
a first irrigation tube connecting the outlet of the EVR system to the first dressing; and a second irrigation tube connecting the inlet of the EVR system to the second dressing.
8. The NPWT dressing assembly of claim 7, wherein a flow path is defined from the outlet, through the first irrigation tube, through the first dressing, through the irrigation tube, through the second dressing, through the second irrigation tube, and to the inlet in order to facilitate flow from the outlet to the inlet.
9. The NPWT dressing assembly of any of claims 1-6, further comprising:
   an EVR system having an inlet and an outlet;
   a first irrigation tube connecting the outlet of the EVR system to the first dressing; and
   a second irrigation tube connecting the inlet of the EVR system to the first dressing.
10. The NPWT dressing assembly of claim 9, wherein the irrigation tube connecting the irrigation pathway of the first dressing to the irrigation pathway of the second dressing comprises at least two lumens.
11. The NPWT dressing assembly of claim 10, wherein a first flow path is defined from the outlet, through the first irrigation tube, through the first dressing, through the second irrigation tube, and to the inlet in order to facilitate flow from the outlet to the inlet.
12. The NPWT dressing assembly of claim 11, wherein a second flow path is defined from the outlet, through the first irrigation tube, through the first dressing, through a first lumen of the irrigation tube, through the second dressing, through the second lumen of the irrigation tube, through the first dressing, and through the second irrigation tube in order to facilitate flow from the outlet to the inlet.
13. The NPWT dressing assembly of any of claims 1-12, further comprising at least one sealing layer comprising an adhesive sized and configured to extend over at least one of the plurality of dressings to contact a peripheral skin surface surrounding at least part of the multi-cavity wound.

### H. Dressing (Expandable)

1. An expandable wound dressing system, the dressing comprising:
   a wound dressing comprising:
      a dressing comprising a transparent polymer and configured to be inserted into a wound; and
      an expander configured to change at least one dimension of the dressing while the dressing is within the wound.
2. The system of claim 1, wherein the dressing comprises a bladder and the expander comprises a pump.
3. The system of claim 1, further comprising a bladder formed within the dressing, and wherein the expander comprises a pump configured to inflate the bladder with a liquid or gas.
4. The system of claim 1, wherein the wound dressing further comprises:
   a central shaft; and
   a plurality of ribs extending radially and orthogonally from the central shaft, the plurality of ribs movably coupled to the central shaft at a distal end of the central shaft;
   wherein the expander is configured to change an angle of the plurality of ribs relative to the central shaft.
5. The system of claim 4, wherein the dressing extends between each of the plurality of ribs.
6. The system of any of claims 1-5, wherein the wound dressing is further configured to provide one or more therapeutic agents to the wound.
7. The system of any of claims 1-6, wherein the wound dressing further comprises a pressure sensor configured to sense a pressure at an edge of the wound.
8. The system of any of claims 1-7, wherein the expander is configured to incrementally collapse the dressing while the dressing is within the wound as the wound heals.
9. The system of any of claims 1-8, wherein the expander is configured to expand the dressing after the dressing is inserted into the wound, so that the dressing is in contact with a wall of the wound.
10. The system of any of claims 1-9, wherein the wound dressing is configured to provide suction to the wound.
11. The system of any of claims 1-10, wherein the expander comprises a motor and an impeller or a fan.
12. The system of any of claims 1-10, wherein the expander comprises a piston, a linkage, and/or a solenoid.

### I. Dressing Expandable A (Umbrella)

1. An expandable wound dressing system configured to be positioned within a wound, the dressing comprising:
   a central shaft; and
   a plurality of ribs extending radially and orthogonally from the central shaft, the plurality of ribs movably coupled to the central shaft at a distal end of the central shaft; and
   an expander operably connected to the plurality of ribs and configured to change an angle of the plurality of ribs relative to the central shaft.
2. The expandable wound dressing system of claim 1, further comprising:
   a wound dressing material spanning between each of the plurality of ribs and an adjacent rib.
3. The expandable wound dressing system of claim 2, wherein the wound dressing material is a transparent polymeric material.
4. The expandable wound dressing system of claim 2 or 3, wherein the wound dressing material further comprises a coating with a therapeutic substance.
5. The expandable wound dressing system of any of claims 1-4, further comprising:
   suction tubing configured to provide suction to a center of the wound.
6. The expandable wound dressing system of any of claims 1-5, further comprising:
   suction tubing configured to provide suction to an edge of the wound.
7. The expandable wound dressing system of any of claims 1-6, further comprising:
   irrigation tubing configured to provide irrigation to the wound.
8. The expandable wound dressing system of any of claim 1-7, wherein during insertion of the wound dressing into the wound, the plurality of ribs are configured to be positioned substantially parallel along the central shaft.
9. The expandable wound dressing system of any of claim 1-7, wherein the expander is configured to be adjusted so as to move the plurality of ribs away from the central shaft to contact one or more walls of the wound.
10. The expandable wound dressing system of any of claim 1-7, the expander is configured to be adjusted so as to move the plurality of ribs back toward the central shaft so as to maintain contact with the one or more walls of the wound.
11. The expandable wound dressing system of any of claims 1-10, further comprising:
   a plurality of stretchers, each of the plurality of stretchers pivotably coupled at a distal end to a rib of the plurality of ribs between a proximal end of the rib and a distal end of the rib; and
   wherein the expander comprises a runner movably coupled to a proximal end of each of the plurality of stretchers, the expander configured to be moved along the central shaft to push the plurality of stretchers into a radial orientation, and wherein the plurality of stretchers in turn push the plurality of ribs into a radially expanded configuration.
12. The expandable wound dressing system of any of claims 1-10, wherein the expander comprises a motor and an impeller or a fan.
13. The expandable wound dressing system of any of claims 1-10, wherein the expander comprises a piston, a linkage, and/or a solenoid.

### J. Dressing Expandable B (Bladder)

1. An expandable wound dressing system, the dressing comprising:
   a bladder comprising a transparent polymer; and
   a pump configured to inflate and deflate the bladder.
2. The expandable wound dressing system of claim 1, wherein the bladder is configured to discourage tissue ingrowth.
3. The expandable wound dressing system of claim 1 or 2, wherein the bladder further comprises a coating with a therapeutic substance.
4. The expandable wound dressing system of any of claims 1-3, wherein the bladder further comprises an internal cavity filled with a therapeutic substance.
5. The expandable wound dressing system of any of claims 1-4, further comprising:
   suction tubing configured to provide suction to a center of the wound.
6. The expandable wound dressing system of any of claims 1-5, further comprising:
   suction tubing configured to provide suction to an edge of the wound.
7. The expandable wound dressing system of any of claims 1-6, further comprising:
   irrigation tubing configured to provide irrigation to the wound.
8. The expandable wound dressing system of any of claims 1-7, wherein the pump is configured to inflate the bladder with a heated or cooled liquid or gas.
9. The expandable wound dressing system of any of claim 1-8, wherein during insertion of the bladder into the wound, the bladder is configured to be in a deflated state.
10. The expandable wound dressing system of any of claim 1-9, wherein the pump is configured to inflate the bladder so that one or more external surfaces of the bladder contact one or more walls of the wound.
11. The expandable wound dressing system of any of claim 1-10, wherein the pump is configured to deflate the bladder so as to maintain contact between the one or more external surfaces of the bladder with the one or more walls of the wound.

### K. Expandable Dressing Method

1. A method of treating a wound using an expandable wound dressing, the method comprising:
   positioning an expandable wound dressing within a wound while the expandable wound dressing is in a collapsed state, the wound defined by one or more walls;
   expanding the expandable wound dressing within the wound to an expanded state; and
   incrementally collapsing the expandable wound dressing as the wound heals.
2. The method of claim 1, wherein in the expanded state, the expandable wound dressing is in contact with the one or more walls of the wound.
3. The method of claim 1 or 2, further comprising:
   monitoring a position of the expandable wound dressing relative to the one or more walls of the wound using a pressure sensor.
4. The method of any of claim 1-3, further comprising:
   providing irrigation to the wound through the expandable wound dressing.
5. The method of any of claim 1-4, further comprising:
   providing suction to the wound through the expandable wound dressing.
6. The method of any of claims 1-5, further comprising:
   monitoring one or more parameters of the wound by observing properties of exudate recovered from the wound.

### L. Octopus Dressing

1. An expandable wound dressing system, the dressing comprising:
   a bladder;
   a pump configured to inflate and deflate the bladder; and
   a plurality of dressings extending around the bladder, wherein each of the plurality of dressings are configured as an elongate arm with a transparent polymeric dressing coupled to a distal end of the elongate arm.
2. The expandable wound dressing system of claim 1, wherein during insertion of the plurality of dressings into the wound, the bladder is configured to be in a deflated state.
3. The expandable wound dressing system of claim 1 or 2, wherein during removal of the plurality of dressings from the wound, the pump is configured to inflate the bladder to pull on the elongate arm of each of the plurality of dressings so as to remove the coupled transparent polymeric dressing from the wound.
4. The expandable wound dressing system of any of claim 1-3, wherein the elongate arms of the plurality of dressings are coupled to one another at a position proximal of the bladder.
5. The expandable wound dressing system of any of claim 1-4, wherein each of the elongate arms comprises irrigation tubing connecting an irrigation source to a corresponding dressing.

### M. Dome

1. A NPWT assembly comprising:
   a suction dome comprising:
      a mounded section;
      a tubing connection configured to be coupled to tubing of a wound contact layer; and
      one or more ports extending from the mounded section, the one or more ports configured to be coupled to an external gas or fluid source.
2. The assembly of claim 1, wherein the one or more ports comprises at least one inlet port and at least one outlet port.
3. The assembly of claim 1 or 2, wherein the dome further comprises at least one filter.
4. The assembly of claim 3, wherein the at least one filter comprises a HEPA filter.
5. The assembly of claim 3 or 4, wherein the at least one filter is configured to filter out microbes.
6. The assembly of any of claims 1-5, wherein the dome further comprises at least one pressure release valve.
7. The assembly of any of claims 1-6, wherein the suction dome is configured such that the one or more ports are positioned at an exterior surface of the sealing layer.
8. The assembly of any of claims 1-7, further comprising a restrictor coupled to at least one of the one or more ports.
9. The assembly of any of claims 1-8, further comprising a resealable cap coupled to at least one of the one or more ports, the resealable cap configured to be opened to allow flow through the at least one of the one or more ports and configured to be closed to prevent flow through the at least one of the one or more ports.
10. The assembly of any of claims 1-9, further comprising at least two suction chambers.
11. The assembly of any of claims 1-10, wherein the NPWT comprises at least two suction domes.
12. The assembly of claim 11, wherein the at least two suction domes are connected in series.
13. The assembly of claim 11, wherein the at least two suction domes are connected in parallel.
14. The assembly of any of claims 1-13, wherein the suction dome is configured to fit by suction into a docking section of the wound contact layer.
15. The assembly of any of claims 1-13, wherein the suction dome is configured to fit by friction into a docking section of the wound contact layer.
16. The assembly of any of claims 1-5, wherein the docking section comprises a shaped trench, the shaped trench configured to facilitate cutting of the sealing layer.
17. The assembly of any of claims 1-16, wherein the docking section comprises a circular wall surrounding a central suction chamber, and a groove formed in a top surface of the circular wall.
18. The assembly of any of claims 1-17, wherein the suction dome comprises a hollow bell-shaped dome configured to fit within the groove formed in the top surface of the circular wall, and at least one irrigation tube extending through each of the one or more ports, the irrigation tube configured to provide inflow and outflow.
19. The assembly of any of claims 1-18, wherein the docking section further comprises a holding tank configured to allow chemical reactions to occur at the wound bed.
20. The assembly of any of claims 1-19, wherein the suction dome includes at least one portion comprising a polymer configured to allow for multiple needle pokes through the at least one portion.

### N. Disposable Dressing

1. A disposable dressing system comprising:
   a wound dressing comprising:
      an adhesive layer, the adhesive layer comprising a transparent material coated on a first side with an adhesive material at least at a peripheral edge, wherein the adhesive material is covered by a removable backing paper;
      a wound contact layer, the wound contact layer comprising a transparent polymeric material having a plurality of passageways therethrough; and
      a port formed through the adhesive layer to the wound contact layer, an inlet of the port in fluid communication with the plurality of passageways of the wound contact layer,
      wherein the wound contact layer is coupled to the adhesive layer at the port and is otherwise separable from the adhesive layer.
2. The system of claim 1, further comprising:
   a filter positioned at the inlet of the port.
3. The system of claim 1 or claim 2, further comprising:
   a resealable flap positioned over the inlet of the port.
4. The system of any of claims 1-3, wherein the port is configured to provide airflow to a wound through the inlet.
5. The system of any of claims 1-3, wherein the port is configured to provide suction to a wound through the inlet.
6. The system of any of claims 1-3, wherein the port is configured to provide irrigation to a wound through the inlet.
7. The system of any of claims 1-6, further comprising:
   a pump system configured to provide suction to a wound through the inlet.
8. The system of claim 7, wherein the pump system is battery-operated.
9. The system of claim 7 or 8, wherein the pump system is configured as a low-flow system.
10. The system of any of claims 7-9, further comprising:
   suction tubing configured to couple the port to the pump system.
11. The system of claim 10, wherein the suction tubing comprises an absorbent material positioned within the tubing, the absorbent material configured to absorb liquids in the tubing.
12. The system of any of claims 1-11, further comprising:
   a collection canister configured to be coupled to the port by suction tubing.
13. The system of claim 12, wherein the collection canister comprises a flexible bag.
14. The system of claim 12 or 13, wherein the collection canister further comprises a passageway lined with an absorbent material, the passageway comprising a plurality of right angled turns.
15. The system of any of claims 1-14, wherein the adhesive layer and the wound contact layer are configured to be separately cut to a size of a wound.

### O. Kit A

1. A kit for treating a wound, the kit comprising:
   a wound dressing comprising any of the wound dressings described above.
2. The kit of claim 1, further comprising:
   a pump.
3. The kit of claim 2, wherein the pump is configured to provide suction to the wound through the wound dressing.
4. The kit of claim 2 or 3, wherein the pump is configured to be battery-operated to provide a low flow-rate.

### P. Kit B

1. A kit for treating a wound, the kit comprising:
   a wound dressing comprising:
      a wound contact layer; and
      an adhesive layer;
      wherein the wound contact layer and the adhesive layer are coupled to one another at least at a first portion of the wound contact layer, and wherein the wound contact layer is separable from the adhesive layer at a second portion of the wound contact layer.
2. The kit of claim 1, wherein at least one of the wound contact layer and the adhesive layer can be cut or torn to a smaller size.
3. The kit of claim 1 or 2, wherein the wound contact layer is separable from the adhesive layer at one end of the wound contact layer while remaining coupled to the adhesive layer at a position of a port.
4. The kit of any of claims 1-3, wherein the wound contact layer comprises one or more perforations, the perforations configured to provide a line along which the wound contact layer can be cut or torn to a smaller size.
5. The kit of any of claims 1-4, wherein the adhesive layer comprises one or more perforations, the perforations configured to provide a line along which the wound contact layer can be cut or torn to a smaller size.
6. The kit of any of claims 1-5, further comprising an outlet port formed in the wound contact layer.
7. The kit of claim 6, wherein the outlet port comprises a lily-pad type port configured to be positioned on the adhesive layer and wound contact layer to provide suction to the wound.
8. The kit of claim 6, wherein the outlet port comprises a docking port extending from the wound contact layer through the adhesive layer.
9. The kit of any of claims 6-8, further comprising a pump configured to be coupled to the outlet port.
10. The kit of claim 9, wherein the pump is configured to provide suction to the wound through the outlet port.
11. The kit of claim 10, further comprising an aperture through the adhesive layer and wound contact layer configured to provide air to the wound.
12. The kit of claim 11, wherein the aperture further comprises a filter configured to filter the air before it enters the wound contact layer.
13. The kit of claim 12, wherein the filter is a HEPA filter.
14. The kit of any of claims 11-13, further comprising a resealable flap positioned over the aperture.
15. The kit of any of claims 1-14, further comprising:
   a collection canister.
16. The kit of claim 15, wherein the collection canister is configured as a solid container.
17. The kit of claim 15, wherein the collection canister is configured as a flexible bag.
18. The kit of claim 15, wherein the collection canister is configured as absorbent material positioned within suction tubing.
19. The kit of any of claims 1-18, further comprising:
   scissors or a scalpel configured to be used to cut the wound dressing to size.
20. The kit of any of claims 1-19, further comprising:
   a bag or other container of saline or another liquid configured to be provided to the wound.
21. The kit of claim 20, wherein the bag or container of saline or another liquid further comprises at least one medicament.

### Q. Low-Pressure NPWT

1. A negative pressure wound therapy system, comprising:
   a vacuum source configured to generate sub-atmospheric pressure of −30 mmHg to −90 mmHg;
   a wound interface dressing comprising a non-compressible, low-durometer material that resists collapse under said pressure; and
   an internal flow distribution structure that maintains tissue-facing surface pressure of +10 mmHg to +50 mmHg,
   wherein the system operates in continuous or intermittent vacuum cycles to preserve tissue perfusion.
2. The system of claim 1, wherein the dressing comprises a lattice, mesh, or scaffold with channels ≥1 mm in diameter.
3. The system of claim 1, wherein the system operates in intermittent vacuum cycles every 1 to 60 minutes.
4. The system of claim 1, wherein pressure sensors embedded within the dressing monitor interface pressure.
5. The system of claim 1, wherein the dressing material has a Shore A durometer of ≤30.
5. The system of claim 1, wherein the system includes a portable, battery-powered vacuum controller.
6. The system of claim 1, wherein the vacuum source is configured to generate a maximum sub-atmospheric pressure followed by generating a minimum sub-atmospheric pressure.
7. The system of claim 6, wherein the maximum sub-atmospheric pressure is between −40 mmHg and −90 mmHg.
8. The system of any of claims 6-7, wherein the minimum sub-atmospheric pressure is between zero and −30 mmHg.
9. The system of any of claims 6-8, wherein the minimum sub-atmospheric pressure is between zero and −5 mmHg.
10. The system of any of claims 6-9, wherein the internal flow distribution structure is configured to maintain a maximum tissue-facing surface pressure followed by maintaining a minimum tissue-facing surface pressure.
11. The system of claim 10, wherein the maximum tissue-facing surface pressure is between +40 mmHg to +50 mmHg.
12. The system of any of claims 10-11, wherein the minimum tissue-facing surface pressure is between zero and +10 mmHg.
13. The system of any of claims 10-12, wherein the minimum tissue-facing surface pressure is between zero and +5 mmHg.
14. A negative pressure wound therapy system, comprising:
   a vacuum source configured to generate sub-atmospheric pressure of zero to −90 mmHg;
   a wound interface dressing comprising a non-compressible, low-durometer material that resists collapse under said pressure; and
   an internal flow distribution structure that maintains tissue-facing surface pressure of zero to greater than +50 mmHg,
   wherein the system operates in continuous or intermittent vacuum cycles to preserve tissue perfusion.
15. A method of treating a wound, comprising:
   applying a non-compressible dressing to the wound;
   applying negative pressure of −30 mmHg to −90 mmHg; and
   maintaining wound surface pressure at +10 mmHg to +50 mmHg to preserve perfusion and avoid compressive ischemia.
16. The method of claim 15, wherein pressure is applied intermittently.
17. The method of claim 15, wherein wound oxygenation is improved relative to conventional foam NPWT.
18. The method of claim 15, further comprising feedback-controlled vacuum modulation based on surface pressure.
19. A method of treating a wound, comprising:
   applying a non-compressible dressing to the wound;
   applying negative pressure of zero to −90 mmHg; and
   maintaining wound surface pressure at zero to greater than +50 mmHg to preserve perfusion and avoid compressive ischemia.

We claim:

1. A disposable dressing system comprising:
a wound dressing comprising:
an adhesive layer, the adhesive layer comprising a transparent material coated on a first side with an adhesive material at least at a peripheral edge, wherein the adhesive material is covered by a removable backing paper;
a wound contact layer, the wound contact layer comprising a transparent polymeric material having a plurality of passageways therethrough; and
a port formed through the adhesive layer to the wound contact layer, an inlet of the port in fluid communication with the plurality of passageways of the wound contact layer,
wherein the wound contact layer is coupled to the adhesive layer at the port and is otherwise separable from the adhesive layer.

2. The system of claim 1, further comprising a filter positioned at the inlet of the port.

3. The system of claim 1, further comprising a resealable flap positioned over the inlet of the port.

4. The system of claim 1, wherein the port is configured to provide airflow to a wound through the inlet.

5. The system of claim 1, wherein the port is configured to provide suction to a wound through the inlet.

6. The system of claim 1, wherein the port is configured to provide irrigation to a wound through the inlet.

7. The system of claim 1, further comprising a pump system configured to provide suction to a wound through the inlet, wherein the pump system is battery-operated.

8. The system of claim 1, further comprising a pump system configured to provide suction to a wound through the inlet, wherein the pump system is configured as a low-flow system.

9. The system of claim 1, further comprising:
a pump system configured to provide suction to a wound through the inlet; and
suction tubing configured to couple the port to the pump system.

10. The system of claim 9, wherein the suction tubing comprises an absorbent material positioned within the tubing, the absorbent material configured to absorb liquids in the tubing.

11. The system of claim 1, further comprising a collection canister configured to be coupled to the port by suction tubing, wherein the collection canister comprises a flexible bag.

12. The system of claim 1, further comprising a collection canister configured to be coupled to the port by suction tubing, wherein the collection canister comprises a passageway lined with an absorbent material, the passageway comprising a plurality of right angled turns.

13. The system of claim 1, wherein the adhesive layer and the wound contact layer are configured to be separately cut to a size of a wound.

14. The system of claim 1, wherein the wound contact layer is configured to be non-absorptive.

15. A negative pressure wound therapy system comprising:
a vacuum source configured to generate sub-atmospheric pressure of −5 mmHg to −90 mmHg;
a wound interface dressing comprising a non-compressible, low-durometer material that resists collapse under the sub-atmospheric pressure; and
an internal flow distribution structure that maintains tissue-facing surface pressure of +5 mmHg to +100 mmHg,
wherein the system operates in continuous or intermittent vacuum cycles to preserve tissue perfusion.

16. The system of claim 15, wherein the wound interface dressing comprises a lattice, mesh, or scaffold with channels ≥1 mm in diameter.

17. The system of claim 15, wherein the system operates in intermittent vacuum cycles every 1 to 60 minutes.

18. The system of claim 15, further comprising one or more pressure sensors embedded within the wound interface dressing, the one or more pressure sensors configured to monitor an interface pressure at a wound surface.

19. The system of claim 15, wherein the non-compressible, low-durometer material has a Shore A durometer of ≤30.

20. The system of claim 15, wherein the vacuum source comprises a portable, battery-powered vacuum controller configured to be coupled to the wound interface dressing.
